SEQUENCE LISTING (110 sequences)

SEQ ID NO: 1 - protein, 10 aa, Homo sapiens
SRSRSRSRSR

SEQ ID NO: 2 - protein, 5 aa, Homo sapiens
SGRSA

SEQ ID NO: 3 - protein, 8 aa, Homo sapiens
VPMSMRGG

SEQ ID NO: 4 - protein, 8 aa, Homo sapiens
IPVSLRSG

SEQ ID NO: 5 - protein, 8 aa, Homo sapiens
RPFSMIMG

SEQ ID NO: 6 - protein, 8 aa, Homo sapiens
VPLSLTMG

SEQ ID NO: 7 - protein, 8 aa, Homo sapiens
VPLSLYSG

SEQ ID NO: 8 - protein, 8 aa, Homo sapiens
IPESLRAG

SEQ ID NO: 9 - protein, 11 aa, Homo sapiens
SGSPAFLAKNR

SEQ ID NO: 10 - protein, 4 aa, Homo sapiens
DAFK

SEQ ID NO: 11 - protein, 10 aa, Homo sapiens
SGKPILFFRL

SEQ ID NO: 12 - protein, 10 aa, Homo sapiens
SGKPIIFFRL

SEQ ID NO: 13 - protein, 8 aa, Homo sapiens
GPLGMLSQ

SEQ ID NO: 14 - protein, 4 aa, Homo sapiens
GGGC

SEQ ID NO: 15 - protein, 4 aa, Homo sapiens
AAAC

SEQ ID NO: 16 - protein, 4 aa, Homo sapiens
SSSC

SEQ ID NO: 17 - protein, 4 aa, Homo sapiens
TTTC

SEQ ID NO: 18 - protein, 19 aa, Homo sapiens
SRSRSRSRSRSGRSAGGGC

SEQ ID NO: 19 - protein, 11 aa, Homo sapiens
KSRSRSRSRSR

SEQ ID NO: 20 - protein, 12 aa, Homo sapiens
KKSRSRSRSRSR

SEQ ID NO: 21 - protein, 20 aa, Homo sapiens
KSRSRSRSRSRSGRSAGGGC

SEQ ID NO: 22 - protein, 21 aa, Homo sapiens
KKSRSRSRSRSRSGRSAGGGC

SEQ ID NO: 23 - protein, 4 aa, Homo sapiens
CGGG

SEQ ID NO: 24 - protein, 4 aa, Homo sapiens
KGGG

SEQ ID NO: 25 - protein, 31 aa, Homo sapiens
HGPEGLRVGFYESDVMGRGHARLVHVEEPHT

SEQ ID NO: 26 - protein, 12 aa, Homo sapiens
GPQGLAGQRGIV

SEQ ID NO: 27 - protein, 12 aa, Homo sapiens
SLLKSRMVPNFN

SEQ ID NO: 28 - protein, 12 aa, Homo sapiens
SLLIFRSWANFN

SEQ ID NO: 29 - protein, 12 aa, Homo sapiens
SGVVIATVIVIT

SEQ ID NO: 30 - protein, 13 aa, Homo sapiens
KGGVPMSMRGGGC

SEQ ID NO: 31 - protein, 13 aa, Homo sapiens
KGGIPVSLRSGGC

SEQ ID NO: 32 - protein, 13 aa, Homo sapiens
KGGVPLSLTMGGC

SEQ ID NO: 33 - protein, 13 aa, Homo sapiens
KGGGSGRSAGGGC

SEQ ID NO: 34 - protein, 12 aa, Homo sapiens
CGGGSGRSAGGC

SEQ ID NO: 35 - protein, 13 aa, Homo sapiens
CGGGSGRSAGGGC

SEQ ID NO: 36 - protein, 11 aa, Homo sapiens
DGGSGRSAGGK

SEQ ID NO: 37 - protein, 3 aa, Homo sapiens
KGG

SEQ ID NO: 38 - protein, 3 aa, Homo sapiens
GGC

SEQ ID NO: 39 - protein, 3 aa, Homo sapiens
GGK

SEQ ID NO: 40 - protein, 2 aa, Homo sapiens
GC

SEQ ID NO: 41 - protein, 11 aa, Homo sapiens
KGGSGRSAGGD

SEQ ID NO: 42 - protein, 3 aa, Homo sapiens
GGD

SEQ ID NO: 43 - protein, 7 aa, Homo sapiens
KGGVPMS

SEQ ID NO: 44 - protein, 6 aa, Homo sapiens
MRGGGC

SEQ ID NO: 45 - protein, 7 aa, Homo sapiens
KGGIPVS

SEQ ID NO: 46 - protein, 6 aa, Homo sapiens
LRSGGC

SEQ ID NO: 47 - protein, 7 aa, Homo sapiens
KGGVPLS

SEQ ID NO: 48 - protein, 6 aa, Homo sapiens
LTMGGC

SEQ ID NO: 49 - protein, 7 aa, Homo sapiens
KGGGSGR

SEQ ID NO: 50 - protein, 6 aa, Homo sapiens
SAGGGC

SEQ ID NO: 51 - protein, 7 aa, Homo sapiens
CGGGSGR

SEQ ID NO: 52 - protein, 5 aa, Homo sapiens
SAGGC

SEQ ID NO: 53 - protein, 6 aa, Homo sapiens
DGGSGR

SEQ ID NO: 54 - protein, 5 aa, Homo sapiens
SAGGK

SEQ ID NO: 55 - protein, 13 aa, Homo sapiens
SRSRSRSRSRSGR

SEQ ID NO: 56 - protein, 6 aa, Homo sapiens
KGGSGR

SEQ ID NO: 57 - protein, 5 aa, Homo sapiens
SAGGD

SEQ ID NO: 58 - protein, 4 aa, Homo sapiens
  MISC_FEATURE (2)..(2): Xaa is any amino acid other than cysteine or lysine
GXGD

SEQ ID NO: 59 - protein, 6 aa, Homo sapiens
  MISC_FEATURE (2)..(4): Xaa is any amino acid other than cysteine or lysine
GXGXGD

SEQ ID NO: 60 - protein, 4 aa, Homo sapiens
  MISC_FEATURE (3)..(3): Xaa is any amino acid other than cysteine or lysine
KGXG

SEQ ID NO: 61 - protein, 6 aa, Homo sapiens
  MISC_FEATURE (3)..(5): Xaa is any amino acid other than cysteine or lysine
KGXGXG

SEQ ID NO: 62 - protein, 4 aa, Homo sapiens
  MISC_FEATURE (3)..(3): Xaa is any amino acid other than cysteine
DGXG

SEQ ID NO: 63 - protein, 6 aa, Homo sapiens
  MISC_FEATURE (3)..(5): Xaa is any amino acid other than cysteine
DGXGXG

SEQ ID NO: 64 - protein, 12 aa, Homo sapiens
CGGGSGRSAGGG

SEQ ID NO: 65 - protein, 13 aa, Homo sapiens
DGGGSGRSAGGGD

SEQ ID NO: 66 - protein, 13 aa, Homo sapiens
DGAGSGRSAGAGD

SEQ ID NO: 67 - protein, 11 aa, Homo sapiens
KGGSGRSAGGG

SEQ ID NO: 68 - protein, 12 aa, Homo sapiens
DGGSGRSAGGGC

SEQ ID NO: 69 - protein, 5 aa, Homo sapiens
SAGGG

SEQ ID NO: 70 - protein, 7 aa, Homo sapiens
DGGGSGR

SEQ ID NO: 71 - protein, 6 aa, Homo sapiens
SAGGGD

SEQ ID NO: 72 - protein, 7 aa, Homo sapiens
DGAGSGR

SEQ ID NO: 73 - protein, 6 aa, Homo sapiens
SAGAGD

SEQ ID NO: 74 - protein, 8 aa, Homo sapiens
GAANLVRG

SEQ ID NO: 75 - protein, 5 aa, Homo sapiens
PRAGA

SEQ ID NO: 76 - protein, 15 aa, Homo sapiens
HHHGAGVPMSMRGAG

SEQ ID NO: 77 - protein, 16 aa, Homo sapiens
HHHGAGIPVSLRSGAG

SEQ ID NO: 78 - protein, 16 aa, Homo sapiens
HHHGAGRPFSMIMGAG

SEQ ID NO: 79 - protein, 16 aa, Homo sapiens
HHHGAGVPLSLTMGAG

SEQ ID NO: 80 - protein, 16 aa, Homo sapiens
HHHGAGVPLSLYSGAG

SEQ ID NO: 81 - protein, 17 aa, Homo sapiens
HHHGAGGAANLVRGGAG

SEQ ID NO: 82 - protein, 20 aa, Homo sapiens
HHHGAGPQGLAGQRGIVGAG

SEQ ID NO: 83 - protein, 14 aa, Homo sapiens
HHHGAGSGRSAGAG

SEQ ID NO: 84 - protein, 21 aa, Homo sapiens
HHHGAGSLLKSRMVPNFNGAG

SEQ ID NO: 85 - protein, 21 aa, Homo sapiens
HHHGAGSLLIFRSWANFNGAG

SEQ ID NO: 86 - protein, 21 aa, Homo sapiens
HHHGAGSGVVIATVIVITGAG

SEQ ID NO: 87 - protein, 12 aa, Homo sapiens
HHHGAGPRAGAG

SEQ ID NO: 88 - protein, 10 aa, Homo sapiens
HHHGAGVPMS

SEQ ID NO: 89 - protein, 5 aa, Homo sapiens
MRGAG

SEQ ID NO: 90 - protein, 10 aa, Homo sapiens
HHHGAGIPVS

SEQ ID NO: 91 - protein, 6 aa, Homo sapiens
LRSGAG

SEQ ID NO: 92 - protein, 9 aa, Homo sapiens
HHHGAGSGR

SEQ ID NO: 93 - protein, 10 aa, Homo sapiens
HHHGAGRPFS

SEQ ID NO: 94 - protein, 6 aa, Homo sapiens
MIMGAG

SEQ ID NO: 95 - protein, 10 aa, Homo sapiens
HHHGAGVPLS

SEQ ID NO: 96 - protein, 6 aa, Homo sapiens
LTMGAG

SEQ ID NO: 97 - protein, 10 aa, Homo sapiens
HHHGAGVPLS

SEQ ID NO: 98 - protein, 6 aa, Homo sapiens
LYSGAG

SEQ ID NO: 99 - protein, 10 aa, Homo sapiens
HHHGAGGAAN

SEQ ID NO: 100 - protein, 7 aa, Homo sapiens
LVRGGAG

SEQ ID NO: 101 - protein, 11 aa, Homo sapiens
HHHGAGPQGLA

SEQ ID NO: 102 - protein, 9 aa, Homo sapiens
GQRGIVGAG

SEQ ID NO: 103 - protein, 12 aa, Homo sapiens
HHHGAGSLLKSR

SEQ ID NO: 104 - protein, 9 aa, Homo sapiens
MVPNFNGAG

SEQ ID NO: 105 - protein, 12 aa, Homo sapiens
HHHGAGSLLIFR

SEQ ID NO: 106 - protein, 9 aa, Homo sapiens
SWANFNGAG

SEQ ID NO: 107 - protein, 12 aa, Homo sapiens
HHHGAGSGVVIA

SEQ ID NO: 108 - protein, 9 aa, Homo sapiens
TVIVITGAG

SEQ ID NO: 109 - protein, 8 aa, Homo sapiens
HHHGAGPR

SEQ ID NO: 110 - protein, 4 aa, Homo sapiens
AGAG

(19) United States
(12) Patent Application Publication
(10) Pub. No.: US 2012/0157824 A1
(43) Pub. Date: Jun. 21, 2012

(54) MRI AND OPTICAL ASSAYS FOR PROTEASES

(75) Inventors: Stefan H. Bossmann, Manhattan, KS (US); Deryl Troyer, Manhattan, KS (US); Matthew T. Basel, Manhattan, KS (US); Thilani Nishanthika Samarakoon, Manhattan, KS (US); Hongwang Wang, Manhattan, KS (US); Viktor Chikan, Manhattan, KS (US); Franklin Orban Kroh, Wamego, KS (US); Olga Barbara Koper, Dublin, OH (US); Brandon Ray Walker, Wamego, KS (US); Xiaoxuan Leaym, Manhattan, KS (US)

(73) Assignees: NANOSCALE CORPORATION, Manhattan, KS (US); KANSAS STATE UNIVERSITY RESEARCH FOUNDATION, Manhattan, KS (US)

(21) Appl. No.: 13/393,472

(22) PCT Filed: Aug. 31, 2010
(86) PCT No.: PCT/US10/47301

§ 371 Date: Feb. 29, 2012

Related U.S. Application Data
(60) Provisional application No. 61/239,313, filed on Sep. 2, 2009.

(51) Int. Cl. A61K 49/18; A61B 5/055

(52) U.S. Cl. 600/420; 424/9.3; 424/9.32

(57) ABSTRACT

The present invention provides multifunctional nanoplatforms for assessing the activity of a protease in vivo or in vitro, along with methods of imaging and detecting the presence of cancerous or precancerous tissues, and the therapeutic treatment thereof, including monitoring of treatment. The diagnostic nanoplatforms comprise nanoparticles and are linked to each other or other particles via an oligopeptide linkage that comprises a consensus sequence specific for the target protease. Cleavage of the sequence by the target protease can be detected using various sensors, and the diagnostic results can be correlated with cancer prognosis. Individual unlinked nanoplatforms are also adaptable for therapeutic hyperthermia treatment of the cancerous tissue.

### CROSS-REFERENCE TO RELATED APPLICATIONS

The present application claims the benefit of and priority from U.S. Provisional Patent Application Ser. No. 61/239,313, filed Sep. 2, 2009, the entire disclosure of which is hereby incorporated by reference herein.

### SEQUENCE LISTING

The following application contains a sequence listing in computer readable format (CRF), submitted as a text file in ASCII format entitled “40884_PCT_SequenceListing.txt,” created on Aug. 24, 2010, as 18 KB. The contents of the CRF are hereby incorporated by reference herein.

### FEDERALLY SPONSORED RESEARCH OR DEVELOPMENT

This invention was made with government support under contract number HHSN261200800059C, awarded by the National Institutes of Health (NIH), and contract number 0930673, awarded by the National Science Foundation (NSF). The United States government has certain rights in the invention.

### BACKGROUND OF THE INVENTION

1. Field of the Invention

The present invention relates to multifunctional nanoplatforms for diagnostic assays, imaging, monitoring, and therapeutic treatment of cancerous tissues.

2. Description of Related Art

### Proteases

A number of proteases are associated with disease progression in cancer, and are known to be over-expressed by various cancer cell lines, as shown in FIG. 1. Examples include Matrix Metalloproteinases (MMPs), Tissue Serine Proteases, and the Cathepsins. Many of these proteases are either upregulated in the cancer cells (i.e., have a much higher activity in the tumor than in healthy tissue), mis-expressed (i.e., are found in compartments where they should not be found), or are involved in embryonic development (but should not be found to any significant extent in an adult cell).

There are 21 different known MMPs that are grouped into families based on their substrates: collagenases, gelatinases, stromelysins, matrilysin, metalloelastase, enamelysin, and membrane-type MMPs. MMPs are usually produced by stromal cells surrounding a tumor, and although not produced by the cancerous cells themselves, are vital to cancer survival and progression for several reasons. First, they cleave cell surface bound growth factors from the stromal and epithelial cells and release them to interact with the cancer cells to stimulate growth. Second, they play a role in angiogenesis by opening the extracellular matrix (ECM) to new vessel development as well as by releasing pro-angiogenic factors and starting pro-angiogenic protease cascades. MMPs play a major role in tumor metastasis by degrading the ECM and the basement membrane (BM), allowing the cancer cells to pass through tissue barriers, leading to cell invasion. They also release ECM and BM fragments, which stimulates cell movement.

Several serine proteases have well-documented roles in cancer as well, especially urokinase plasminogen activator (uPA) and plasmin. Elevated expression levels of urokinase and several other components of the plasminogen activation system have been found to be correlated with tumor malignancy. uPA is a very specific protease that binds to its receptor, uPAR, and cleaves the inactive plasminogen (a zymogen) to the active plasmin. This is the first step in a well-known cascade that causes angiogenesis in tumors. It is believed that the tissue degradation that follows plasminogen activation facilitates tissue invasion and contributes to metastasis. Plasmin is a somewhat non-specific protease that goes on to cleave proteins or peptides including activating procollagenases, degrading the ECM, and releasing/activating growth factors. Although plasmin is somewhat non-specific and a consensus sequence is hard to determine, uPA does have a well-defined consensus sequence.

Cathepsins, with a few exceptions, are cysteine proteases. Often found in the lysosomal/endosomal pathway, cathepsins usually operate at low pH values, but some are still active at neutral pH. Three of the cathepsins, B, D, and L, are active at neutral pH and are often misexpressed in cancer, causing activation outside of the cells. This activation outside of the cell can cause ECM degradation.

### Magnetic Resonance Imaging

Magnetic Resonance Imaging (MRI) is a non-invasive diagnostic tool to obtain images of the inside of a body. It provides information about pathological alterations, such as tumors, of living tissues (medical imaging). MR images are based on the spin-relaxation times of protons (¹H), excited using radio frequency (RF) pulse patterns in an external magnetic field. The variation of the T₁-relaxation (spin-lattice or longitudinal relaxation time) and T₂-relaxation (spin-spin or transverse relaxation time) times generates image contrasts between different tissues and pathologies depending upon how the MR image is collected. More specifically, when a patient is placed within the magnetic field (B₀) of the MR magnet of the apparatus, the protons of the body line up in the direction of the external field (B₀). In addition, the magnetic axis of each proton starts to rotate (precess) around the direction of this field. Some of these protons precess with their magnetic moments aiming in a direction closely parallel to the external magnetic field, while others precess with their magnetic moments aiming close to anti-parallel to the field. This creates a net magnetic moment in the tissues of the patient, with the tissue magnetism (M) oriented exactly parallel to the external field (B₀). Short radio frequency (RF) pulses are transmitted into the patient at different angles changing the orientation of the proton magnetic moments, inducing an electric current in a receiver coil located outside of the patient's body. These signals are used to reconstruct the MR image.

To reconstruct an image, several MR signals are needed, and several pulses must be transmitted. Between the pulse transmissions, the protons undergo two different relaxation processes: T₁ and T₂ relaxation. The MRI operator determines whether the tissue contrast will be determined mainly by differences in T₁ (T₁-weighted image) or T₂ (T₂-weighted image) by modifying the pulse sequence and timing. For example, for T₁-weighted images, tissues exhibiting a strong magnetism will induce strong signals and generally appear bright in the image, while tissues exhibiting weak magnetism will induce weak signals and appear dark. Pulse sequences are performed by computer programs that control the hardware aspects of the MRI measurement process. T₁ is defined as the time until the proton magnetization has regained 63% of its original value. The T₁ relaxation time is a measure of the time that the excited ¹H nuclei require to realign with the external magnetic field. In general, T₁ is longer in tissues having either smaller, more mobile molecules (i.e., fluids) or larger, less mobile molecules (i.e., solids), while T₁ is shortest in tissues having molecules of medium size and mobility (i.e., fat). T₂ relaxation is caused by energy exchange of the excited protons and nearby magnetic nuclei (¹H, and less importantly, ¹³C, and ¹⁵N). T₂-weighted imaging relies on local dephasing (loss of phase coherence) of spins oriented at an angle to the external field following the transmission of the RF pulse. T₂ is defined as the time when the magnetization (M_{xy}) has lost 63% of its original value. Fluid and fluid-like tissues typically have a long T₂ (MR signal disappears slowly), and solid tissues and substances have a short T₂. The T₂* (also called T₂star) relaxation time possesses two additive components, the T₂ relaxation time and the contribution of local magnetic field non-uniformities to the total relaxation. In the absence of an externally applied pulse, the T₂* effect can cause rapid loss in coherence, and therefore loss of transverse magnetization and the MRI signal. Based on its definition, T₂* is always shorter than T₂.

M_{z}(t)=M_{z,eq}−[M_{z,eq}−M_{z(0)}]e^{-t/T}^{₁}

M_{z}(t): z-component of the nuclear spin magnetization

M_{z,eq}: thermal equilibrium value of M_{z}

M_{xy}(t)=M_{xy}e^{-t/T}^{₁}

M_{xy}(t): component of M that is perpendicular to B₀

$$\frac{1}{{T}_{2} *} = \frac{1}{{T}_{2}} + \frac{1}{{T}_{in homogenous}} = \frac{1}{{T}_{2}} + λγ Δ {B}_{0}$$

γ: gyromagnetic ratio

ΔB₀: difference in strength of the locally varying field

Paramagnetic and superparamagnetic MRI contrast agents (such as magnetic nanoparticles, “MNP s”) can be used to change the signal intensity of the tissue being imaged by altering the T₁ and/or T₂ relaxation times of the ¹H nuclei in the tissue. In general, positive contrast agents cause a reduction in the T₁ relaxation time (increased signal intensity on T₁ weighted images), and appear bright on MR images. Negative contrast agents result in shorter T₁ and T₂ relaxation times, and appear predominantly dark on MRI. The most common MRI contrast agents are based on organic chelates of gadolinium cations. Although less toxic than iodinated contrast agents (commonly used in X-ray or CT), gadolinium agents have been linked to nephrogenic systemic fibrosis when used in some dialysis patients. In addition, gadolinium contrast agents require direct contact with the in vivo water to be activated. Small particles of iron oxides are also used as superparamagnetic contrast medium in MRI. These agents exhibit strong T₁ relaxation properties, and due to susceptibility differences to their surrounding, also produce a strongly varying local magnetic field which enhances T₂ and T₂* relaxations of the ¹H spins in the tissue. Small Particle Iron Oxide Nanoparticles (SPIONs) of less than 300 nm can remain intravascular for several hours and thus can serve as blood pool agents. However, they can also be quickly taken up by the reticuloendothelial system and become distributed among healthy tissue and accumulate in the liver. They also tend to clump together into ineffective sizes. Aqueous dispersions of single, stabilized sub-20 nm nanocrystals (hydrodynamic size) of iron oxides are classified as ultrasmall particles of iron-oxide (USPIO). Typically, these materials generate positive contrasts in T₁-weighted MR images and negative contrasts in T₂-weighted images. Typical relaxivities for aqueous USPIO dispersions are r₁=10-20 mM⁻¹s⁻¹ for T₁-enhancement, and r₂=approx. −100 mM⁻¹s⁻¹ for T₂-decrease in clinical MRI fields of 60-100 MHz (1.4 to 2.35 T). The relaxivities r₁ and r₂ are measures of the ability of the agent to enhance or decrease, respectively, the longitudinal or transversal relaxations of the proton spins in the tissue.

$${r}_{1} = \frac{{T}_{1 , contrast}^{- 1} - {T}_{1 , water}^{- 1}}{c ( Fe )}$$

$${r}_{2} = \frac{{T}_{2 , contrast}^{- 1} - {T}_{2 , water}^{- 1}}{c ( Fe )} ,$$

where c(Fe): mM, T₁, T₂: s.

One commercial iron oxide MRI contrast agent is Feridex® (Bayer HealthCare), which consists of a γ-Fe₂O₃-core of 4-5 nm in diameter and a dextran coating.

### Light Backscattering

Surface Plasmon Resonance (SPR) occurs when an electromagnetic wave interacts with the conduction electrons of a metal. The periodic electric field of the electromagnetic wave causes a collective oscillation of the conductance electrons at a resonant frequency relative to the lattice of positive ions. Light is absorbed or scattered at this resonant frequency. The process of absorption is characterized by the conversion of incident resonant photons into photons or vibrations of the metal lattice, whereas scattering is the re-emission of resonant photons in all directions. Because of these two processes, the experimentally observable SPR peak of any metal nanostructure features both absorption and scattering components. Gustav Mie was the first scientist to develop a method to calculate the SPR spectra of (noble) metal nanostructures by solving Maxwell's equation for spherical nanoobjects. The “Mie”-theory has been extended stepwise for a variety of objects with simple geometries, such as spheroids and rods. However, exact solutions to Maxwell's equations have been found only for spheres, concentric spherical shells, spheroids, and infinite cylinders. Therefore, approximation is required to solve the equations for other geometries. The discrete dipole approximation (DDA) is the preferred method of choice in the art, because it can be easily adapted to any geometry.

The optical extinction E(λ) of nanoparticles being smaller than the wavelength of the exciting light source, is:

E(λ)=S(λ)+A(λ)

where λ is the wavelength, S is scattering, and A is absorbance. The extinction efficiency factor Qₑₓₜ, which is the sum of the scattering efficiency factor Q_{sca} and the absorption efficiency factor Q_{abs}, is defined as the quotient of Cₑₓₜ and the physical cross-section area πR². The scattering and absorption efficiency factors can be calculated according to the general Mie theory, which is explained, in some detail, below. Both can be expressed as infinite series:

$$\begin{matrix}{Q}_{ext} = \frac{2}{{x}^{2}} {\sum }_{n = 1}^{∞} ( 2 n + 1 ) Re [ {a}_{n} + {b}_{n} ] & {a}_{n} = \frac{m {Ψ}_{n} ( mx ) {Ψ}_{n}^{′} ( x ) - {Ψ}_{n} ( x ) {Ψ}_{n}^{′} ( mx )}{m {Ψ}_{n} ( mx ) {ξ}_{n}^{′} ( x ) - m {ξ}_{n} ( x ) {Ψ}_{n}^{′} ( mx )} \\ {Q}_{sca} = \frac{2}{{x}^{2}} {\sum }_{n = 1}^{∞} ( 2 n + 1 ) [ {a}_{n}^{2} + {b}_{n}^{2} ] & {b}_{n} = \frac{{Ψ}_{n} ( mx ) {Ψ}_{n}^{′} ( x ) - m {Ψ}_{n} ( x ) {Ψ}_{n}^{′} ( mx )}{{Ψ}_{n} ( mx ) {ξ}_{n}^{′} ( x ) - m {ξ}_{n} ( x ) {Ψ}_{n}^{′} ( mx )} \\ {Q}_{abs} = {Q}_{ext} - {Q}_{sca} & x = \frac{2 π {n}_{m} R}{λ}\end{matrix}$$

Re denotes the real part of the refractive index, m is the ratio of the refractive index of the spherical nanoparticle n to that of the surrounding medium nₘ, while x is the size parameter. λ is the incident wavelength, R is the diameter of the nanoparticle. Ψₙ and Σₙ and are the Riccati-Bessel functions. The prime represents the first differentiation with respect to the argument in parentheses.

A(λ)=ε_{abs(λ)}cl E(λ)=(ε_{abs(λ)}+ε_{sca(λ)})cl=(ε_{ext(λ)})cl

A(λ) is the absorbance or optical density of the sample, ε (M⁻¹ cm⁻¹) is the molar absorption (ε_{abs}), scattering (ε_{sca}) or extinction coefficient (εₑₓₜ), c(M) is the concentration of the light absorbing and scattering species and λ(cm) is the optical path length.

The molar absorption and scattering coefficients are directly related to the absorption and scattering cross-section by means of the following equation:

$${ɛ}_{ext} = \frac{{N}_{A} {C}_{ext}}{0.2303}$$

where N_{A} is Avogadros number. Metal nanoparticles show remarkably larger absorption cross-sections compared to organic dyes and metal complexes. A typical example is the nanospheres that have been used for the laser-induced photothermal hyperthermia treatment of cancer cells, which feature an absorption cross-section of 2.93×10⁻¹⁵ m² (ε=7.66×10⁹ M⁻¹ cm⁻¹) at their plasmon resonance maximum of λ=528 nm. This is five orders of magnitude larger than of the commonly used NIR dye indocyanine green (ε=1.08×10⁴M⁻¹ cm⁻¹ at λ=778 nm) or the sensitizer ruthenium(II)-tris-bipyridine (1.54×10⁴ at M⁻¹ cm⁻¹ at λ=452 nm) and four orders of magnitude larger than rhodamine-6G (ε=1.16×10⁵ M⁻¹ cm⁻¹ at λ=530 nm) or malachite green (ε=1.49×10⁵ M¹ cm⁻¹ at λ=617 nm). Metal nanoparticles possess remarkable light scattering properties as well. Gold nanospheres of 80 nm in diameter have approximately the same Mie-scattering characteristics than polystyrene beads of 300 nm (both feature C_{sca}=1.23×10¹⁴ m² at λ=560 nm, corresponding to a molar scattering coefficient of 3.22×10¹⁰ M⁻¹ cm⁻¹). This strong scattering is five orders of magnitude higher than the light emission (fluorescence) from fluoresceine (ε=9.23×10⁴M⁻¹ cm⁻¹ at λ=−521 nm, emission quantum yield Φ=0.98 at λ=483 nm).

There is a need in the art for improved methods of quantitatively detecting cancer progression and stages of the disease that can be applied in vitro and in vivo. There also is a need for in vivo characterization of cancer, so that treatment can be directed to the most malignant cancer tissue. There is also a need for in vivo imaging of cancerous tissue location and extension in all parts of the body, including the brain, which can be performed and observed in real-time resolution.

### SUMMARY OF THE INVENTION

The present invention provides nanoplatforms and nanoplatform assemblies for detecting protease activity. The assemblies comprise a first nanoplatform comprising a first nanoparticle and a protective layer, a second nanoplatform comprising a second nanoparticle and a protective layer, and an oligopeptide linkage between the first and second nanoplatforms. The linkage comprises a protease consensus sequence. In addition, at least one of the first or second nanoplatforms further comprises a functional group selected from the group consisting of porphyrins, chlorins, bacteriochlorins, phthalocyanines, biotin, derivatives thereof, and combinations thereof.

The invention also provides a composition comprising a diagnostic assay including the inventive nanoplatform assembly and a pharmaceutically-acceptable carrier.

A method for detecting the activity of a protease associated with a cancerous or precancerous cell in a mammal is also provided. The method comprises contacting a fluid sample from the mammal with a diagnostic assay comprising the inventive nanoplatform assembly. The assay is then exposed to an energy source, and changes in the optical extinction of the assay are detected. These changes correspond to protease activity.

A further method for detecting the activity of a protease associated with a cancerous or precancerous cell in a mammal is also provided. The method comprises administering to the mammal a composition comprising a diagnostic assay including the inventive nanoplatform assembly and a pharmaceutically-acceptable carrier. The assay is then located in a region of interest in the mammal suspected of having a cancerous or precancerous cell. The region is then exposed to an energy source, and the backscattering spectrum of the assay is detected.

In a further aspect, the invention provides an MRI imaging method for detecting the activity of a protease associated with a cancerous or precancerous cell in a mammal. The method comprises administering to the mammal a composition comprising a diagnostic assay including the inventive nanoplatform assembly and a pharmaceutically-acceptable carrier. The assay is then located in a region of interest in the mammal suspected of having a cancerous or precancerous cell. Radio frequency pulses are transmitted to the region of interest, and MR image data comprising T₁ and T₂ values, is then acquired.

An additional MRI imaging method for detecting the activity of a protease associated with a cancerous or precancerous cell in a mammal is also provided. The method comprises administering to the mammal a diagnostic assay including the inventive nanoplatform assembly and a pharmaceutically-acceptable carrier, wherein the assembly linkage comprises the protease consensus sequence SGRSA (SEQ ID NO: 2). The assay is then located in a region of interest in the mammal suspected of having a cancerous or precancerous cell. Radio frequency pulses are transmitted to the region of interest, and MR image data comprising T₁ and T₂ values, is then acquired. Depending upon the results of this assay, the imaging method is repeated using other specific consensus sequences.

The invention also provides a therapeutic nanoplatform comprising a first nanoparticle and a protective layer surrounding the nanoparticle. The protective layer is selected from the group consisting of siloxane nanolayers, ligand monolayers, and combinations thereof.

A composition comprising a diagnostic assay including the inventive nanoplatform and a pharmaceutically-acceptable carrier is also provided.

The invention also provides a method of inhibiting the growth of cancerous or precancerous cells in a mammal. The method comprises administering to the mammal the composition comprising a diagnostic assay including the inventive therapeutic nanoplatform and a pharmaceutically-acceptable carrier. The assay is then located in a region of interest in the mammal suspected of having a cancerous or precancerous cell. The nanoplatform is then heated using magnetic A/C-excitation, whereby the tissue in the region of interest is heated to a temperature of at least about 40° C.

The invention is also concerned with therapeutic nanoplatforms for inhibiting the growth of cancerous or precancerous cells in a mammal by magnetic A/C-excitation of the nanoplatforms, thereby heating the cancerous or precancerous cells.

Inventive MRI contrast agents are also provided in the invention. The agents comprise a core/shell nanoparticle having an iron core. The MRI contrast agents have an r₁ of greater than about 100 mM⁻¹s⁻¹ for T₁-enhancement and an r₂ with an integer number greater than about −2,000 mM⁻¹s⁻¹ for T₂-decrease.

The invention is also concerned with a further nanoplatform assembly for monitoring progression of cancer treatment in a mammal. The assembly comprises a nanoplatform comprising a first nanoparticle and a protective layer, a particle, and an oligopeptide linkage between the nanoplatform and the particle. The linkage comprises a protease consensus sequence. The method comprises contacting a first fluid sample from the mammal with a first diagnostic assay comprising the nanoplatform; exposing the first assay to an energy source; and detecting the changes in the absorption or emission spectrum of the first assay over time relative to the absorption or emission spectrum of the first assay prior to contact with the first fluid sample, wherein the changes correspond to a first level of protease activity in the first sample. This process is repeated at a later stage during cancer treatment and the subsequent protease activity levels are compared to the initial (or first) protease levels. Based upon changes in the protease activity levels, a determination is then made to increase, decrease, or change the method of treatment.

### BRIEF DESCRIPTION OF THE DRAWINGS

FIG. 1 depicts the four main stages of cancer progression and the proteases associated with these stages;

FIG. 2 illustrates biotin labeling using a statistical mix of dopamine-anchored stealth ligands and biotinylated dopamine-anchored stealth ligands to the amino-terminated siloxane protection layer around the Fe/Fe₃O₄-nanoparticle using CDI;

FIG. 3 is an illustration of the cleavage of two nanoplatforms comprising a Fe/Fe₃O₄-nanoparticle with a stealth ligand coating featuring chemically attached porphyrins linked with a urokinase cleavage sequence;

FIG. 4 illustrates an alternative linking method utilizing a porphyrin as part of the linkage between two nanoplatforms;

FIG. 5 illustrates an alternative assembly method whereby the ligands are pre-linked using a cleavage sequence before being bound to the nanoparticle surface;

FIG. 6 depicts a reaction scheme for synthesizing Ligand A according to the procedures described in Example 3;

FIG. 7 depicts the attachment of a porphyrin compound to Ligand from Example 3;

FIG. 8 shows a reaction scheme for attaching biotin labels to the nanoplatforms;

FIG. 9 illustrates an alternative method for stealth ligand linking prior to attachment to the nanoparticles;

FIG. 10 is a graph of the T₁ relaxation times of Fe/Fe₃O₄ Nanoparticles without (A) and with (B) ligand stabilization, from Example 11;

FIG. 11 shows the T₂ relaxations times of Fe/Fe₃O₄ Nanoparticles without (A) and with (B) ligand stabilization, from Example 11;

FIG. 12 illustrates that the decrease of −(r₂/r₁) follows approximately a pseudo first order kinetics, as calculated in Example 11;

FIG. 13 shows the relative fluorescence of Fe/Fe₃O₄-Nanoplatform featuring “free” sodium tetracarboxylate porphyrin (TCPP) (i) and zinc-doped sodium tetracarboxylate porphyrin (ii) from Example 12;

FIG. 14 depicts the fluorescence intensities of Fe/Fe₃O₄-nanoparticles featuring zinc-doped sodium TCPP and sodium TCPP from Example 12;

FIG. 15 shows the fluorescence of the Fe/Fe₃O₄ nanoplatform as the concentration of unbound sodium TCPP in PBS is increased in Example 12;

FIG. 16 illustrates fluorescence microscopy of the Fe/Fe₃O₄ nanoplatform with tethered porphyrins from Example 12;

FIG. 17 illustrates the data from the assay in urine from rats impregnated with MATB III type cancer cells using the light switch-based sensor in Example 13;

FIG. 18 shows the plot of the relative intensities of the luminescence of TCPP occurring at λ=656 nm using the data from FIG. 17;

FIG. 19 illustrates the single-photo-counting spectra, from the right and left limbs of the mice from Example 14 recorded through a fluorescence microscope;

FIG. 20 is a graph of the observed protease cleavage kinetics as a function of protease (urokinase) concentration from Example 15;

FIG. 21 shows the UV/Vis backscattering spectrum of a nanoparticle-dimer in water in the presence of urokinase from Example 16;

FIG. 22 is a graph showing the changes in the optical extinction over time from Example 16;

FIG. 23 illustrates a plot of the optical extinction at 440 nm divided by the optical extinction at 600 nm over time from Example 16;

FIG. 24 illustrates the UV/Vis spectrum of the “free” and Fe/Fe₃O₄-attached tetracarboxyphenyl porphyrin (TCPP), together with the zinc complexes of the porphyrin in H₂O at a concentration of 7.5×10⁻⁶ M from Example 17;

FIG. 25 is an MRI image of two mice from Example 19;

FIG. 26 illustrates the average tumor volume (mm³) from the hyperthermia tumor inhibition and control studies from Example 20;

FIG. 27 is a graph of change in temperature over time for the hyperthermia tests for various nanoparticles and nanoplatforms from Example 21;

FIG. 28 depicts the calculated specific absorption rates for various Fe and FeO₃ nanoparticles as a function of average particle diameter from Example 22;

FIG. 29 is a graph showing the calculated specific absorption rates as a function of the shape of the magnetic field used for the hyperthermia treatments;

FIG. 30 illustrates the available surface area of spherical nanoparticles for ligand binding as a function of their diameter from Example 24;

FIG. 31 shows the number of dopamine-anchored ligands per nanoparticle as a function of the nanoparticle diameter from Example 24;

FIG. 32 illustrates the effect of variations in the nanoparticle diameter on the number of ligands that form a monolayer on the nanoparticle surface from Example 24;

FIG. 33 is a graph of the results from the in vitro monitoring of cancer treatment from Example 25;

FIG. 34 is a graph showing the effect of the nanoparticles on neural stem cell (NSC) viability from Example 26;

FIG. 35 is a graph showing the effect of the nanoparticles on B16F10 cancer cell viability from Example 26;

FIG. 36 is a bright field image of NSCs loaded with the Fe/Fe₃O₄ nanoplatform from Example 26 showing positive Prussian blue staining for presence of iron and counterstained with nuclear fast red;

FIG. 37 is a Transmission electron microscopy image of and NSC loaded with Fe/Fe₃O₄ nanoplatforms from Example 26 (magnification 30,000×);

FIG. 38 is a graph showing the loading efficiency of the Fe/Fe₃O₄ nanoplatforms from Example 26, based upon Fe concentration per NSC cell loaded with various concentrations of the nanoplatforms, where “*” indicates statistically significant results (p-value less than 0.05) when compared with control;

FIG. 39 is a graph showing temperature measurements after AMF of NSCs loaded with the Fe/Fe₃O₄ nanoplatforms from Example 26, and NSC controls at the pellet and in the agarose solid, where “*” indicates statistically significant results (p-value less than 0.1) when compared with control;

FIG. 40(A)-(F) (A-D) are images of tissue sections of melanoma tumor bearing mice from Example 26;

FIG. 41 is a graph comparing tumor volumes in mice injected with B16-F10 melanoma cells and saline without AMF with mice injected with B16-F10 and nanoparticle-loaded NSCs (with or without AMF treatment) from Example 26;

FIG. 42(A)-(B) are images of 2-D gels of melanoma tissues from mice treated with saline+AMF (A) or nanoparticle-loaded NSCs+AMF (B) from Example 26;

FIG. 43 is a table of the identified proteins of melanoma tissues from mice treated with saline+AMF or nanoparticle-loaded NSCs+AMF from Example 26;

FIG. 44 is a schematic depicting the formation of nanoplatform assemblies using Au-coated nanoplatforms and oligopeptide SEQ ID NO: 66 (deleted at the N-terminus by 1 residue and the C-terminus by 2 residues), as described in Example 27;

FIG. 45 is a graph of the results of the stability tests from Example 27;

FIG. 46 is a graph of the loading efficiency of the Au-coated nanoplatforms from Example 27, where the black circles indicate the Fe uptake (in pg Fe/cell) by the B16F10 cancer cells, the squares indicate the Fe uptake (in pg Fe/cell) by the stem cells, and the triangles indicate the Fe uptake (in pg Fe/cell) by the MS-1 epithelial cells, as a function of Fe concentration in the culture medium;

FIG. 47 is a schematic of multi-plexing nanoplatforms using multiple cyanine dyes on a central stealth-coated nanoparticle for detection of multiple proteases simultaneously;

FIG. 48 is a graph of the emission spectra of various cyanine dyes;

FIG. 49 is a schematic depicting oligoplexing of nanoparticles from Example 28;

FIG. 50 is an image of monocytes/macrophages loaded with nanoparticles from Example 29;

FIG. 51(A)-(D) are MRI images using the nanoplatform imaging agents in mice bearing B16F10 metastasizing lung melanomas from Example 30;

FIG. 52 is an image of mice 1 hour after being injected with the light switch nanoplatform using cyanine dyes from Example 31;

FIG. 53 is an image of mice 2 hours after being injected with the light switch nanoplatform using TCPP and rhodamine chromophores from Example 31;

FIG. 54 is an image of mice 24 hours after being injected with the light switch nanoplatform using TCPP and rhodamine chromophores from Example 31; and

FIG. 55 is a graph of the XRD data from Example 26.

### DETAILED DESCRIPTION

The present invention provides diagnostic, imaging, and therapeutic nanoplatforms and methods of using the same. Nanoplatforms are nanoscale 100 nm) structures designed as general platforms to create a variety of multitasking theranostic (diagnostic and therapeutic) devices and assays. The inventive nanoplatforms comprise an inorganic nanoparticle core with one or more protective layers. The inorganic core preferably comprises a core/shell nanoparticle. The protective layer is preferably selected from the group consisting of siloxane nanolayers, ligand monolayers, and combinations thereof. Gold coatings can also be used in addition to the protective layers. The nanoplatforms can further comprise chemically attached functional groups (i.e., molecules or compounds) bound to the protective layer. These functional groups preferably localize in, and are selectively taken up by tissues, and preferably target cancerous tissues. The protective layers and functional groups can also be utilized to modify properties of the nanoplatform, such as solubility. Preferred functional groups are selected from the group consisting of porphyrins, chlorins, bacteriochlorins, phthalocyanines, biotin, derivatives thereof, and combinations thereof.

In some embodiments, the functional groups will be bound directly to the protective layer. In other embodiments, the functional groups will be attached to the monolayer via oligopeptide linkages, which are selectively cleaved by a protease in the target tissue. Two or more nanoplatforms can also be linked together via these oligopeptide linkages. The nanoplatforms can also be linked to particles, such a chromophores and dyes via these oligopeptide linkages. In further embodiments, porphyrin compounds can be used in conjunction with oligopeptide linkages to link two nanoplatforms. It will be appreciated that the particular combination of the components of these multifunctional nanoplatforms can be adapted for diagnostic imaging, detection, monitoring, and therapeutic treatment of cancerous tissues.

### Inorganic Nanoparticle Core

As previously noted, the nanoplatforms preferably comprise an inorganic core, which comprises a nanoparticle. The term “nanoparticle” as used herein refers to metal particles with sizes under 100 nm. Preferred nanoparticles will be bimagnetic and comprise a metal or metal alloy core and a metal shell. Preferred cores are selected from the group consisting of Au, Ag, Cu, Co, Fe, and Pt. Even more preferably, the nanoparticles feature a strongly paramagnetic Fe core. Preferred shells are selected from the group consisting of Au, Ag, Cu, Co, Fe, Pt, the metal oxides (e.g., FeO, Fe₃O₄, Fe₂O₃, Fe_{X}O_{y}. (non-stoichiometric iron oxide), CuO, Cu₂O, NiO, Ag₂O, Mn₂O₃) thereof, and combinations thereof A particularly preferred nanoparticle is a superparamagnetic Fe/Fe₃O₄ core shell nanoparticle. Suitable nanoparticles are available from NanoScale® Corporation, Manhattan, Kans., including without limitation, those available under the name NanoActive®.

The nanoparticles preferably have an average total diameter of from about 3 nm to about 100 nm, more preferably from about 5 nm to about 20 nm, and even more preferably from about 7 nm to about 10 nm. The core of the nanoparticle preferably has a diameter of from about 2 nm to about 100 nm, more preferably from about 3 nm to about 18 nm and more preferably from about 5 nm to about 9 nm. The metal shell of the core/shell nanoparticle preferably has a thickness of from about 1 nm to about 10 nm, and more preferably from about 1 nm to about 2 nm. The nanoparticles also preferably have a Brunauer-Emmett-Teller (BET) multipoint surface area of from about 20 m²/g to about 500 m²/g, more preferably from about 50 m²/g to about 350 m²/g, and even more preferably from about 60 m²/g to about 80 m²/g. The nanoparticles preferably have a Barret-Joyner-Halenda (BJH) adsorption cumulative surface area of pores having a width between 17.000 Å and 1000,000 Å of from about 20 m²/g to about 500 m²/g, and more preferably from about 50 m²/g to about 150 m²/g. The nanoparticles also preferably have a BJH desorption cumulative surface area of pores having a width between 17.000 Å and 3000.000 Å of from about 50 m²/g to about 500 m²/g, and more preferably from about 50 m²/g to about 150 m²/g. The nanoparticle population is preferably substantially monodisperse, with a very narrow size/mass size distribution. More preferably, the nanoparticle population has a polydispersity index of from about 1.2 to about 1.05. It is particularly preferred that the nanoparticles used in the inventive nanoplatforms are discrete particles. That is, clustering of nanocrystals (i.e., nanocrystalline particles) is preferably avoided.

### Protective Layers

The inorganic core is preferably coated with one or more protective layers. In one aspect, the nanoparticle is coated with an organo-functional siloxane protecting layer, and more preferably an aminofunctional siloxane (ASOX) layer. The siloxane layer preferably protects the core from biocorrosion under physiological conditions. Preferred aminofunctional siloxanes are selected from the group consisting of 3-aminopropyltriethoxysilane, 3-aminopropyltrimethoxysilane, 3-(trimethoxysilyl)propanenitrile, and 3-(triethyoxysilyl)propanenitrile. Suitable siloxanes can be purchased, or they can be synthesized via known methods (i.e., aminolysis of chloroalkyltrimethoxysilanes or hydrogenation of cyanoalkyltrimethoxysilanes). The thickness of the siloxane layer can be modified depending upon the end use and the amount of time the nanoplatform will remain in vivo. Preferably, the nanoplatform comprises an iron-containing nanoparticle coated with an aminosiloxane layer. Depending on the thickness of the aminosiloxane layer, the iron-containing nanoparticle will preferably biocorrode within about 2 days to about 2 weeks, releasing iron-cations. Advantageously, these iron cations will enhance oxidative damage to the tumor tissue via iron(II/III)-enhanced chemistry of reactive oxygen species (ROS). Whereas the classic “stealth” ligand layer (discussed below) will affect biocompatibility, the optimal thickness of the protective aminosiloxane layer will control the kinetics of iron(II/III)-release from the bimagnetic nanoparticle nanoplatforms.

For complexation of the nanoparticle dimers and stabilization of the nanoparticle assemblies, the nanoparticles are preferably “stealth” coated or stabilized with a layer of ligands. Stabilized nanoparticles preferably comprise a protective layer surrounding the nanoparticle. The stealth coating can be attached directly to the nanoparticle, or may be added as a second monolayer surrounding the siloxane protecting layer. For example, a preferred combination is an aminosiloxane layer surrounded by a dopamine-stealth ligand layer. The term “stabilized” as used herein means the use of a ligand shell to coat, protect, or impart properties to the nanoparticle. The stealth coating enables the nanoplatforms to avoid the reticuloendothelial system, and enables the use of the nanoplatforms within a mammal for at least 2 days, and preferably from about 2 days to about 14 days for diagnosis and treatment.

The ligands comprise functional groups that are attracted to the nanoparticle's metal surface. Preferably, the ligands comprise at least one group selected from the group consisting of thiols, alcohols, nitro compounds, phosphines, phosphine oxides, resorcinarenes, selenides, phosphinic acids, phosphonic acids, sulfonic acids, sulfonates, carboxylic acids, disulfides, peroxides, amines, nitriles, isonitriles, thionitriles, oxynitriles, oxysilanes, alkanes, alkenes, alkynes, aromatic compounds, and seleno moieties. Preferred protective layers are selected from the group consisting of alkanethiolate monolayers, aminoalkylthiolate monolayers, alkylthiolsulfate monolayers, and organic phenols (e.g., dopamine and derivatives thereof). A particularly preferred class of ligands comprises oligoethylene glycol units with dopamine-based anchors. The thickness of the ligand layer can be tailored depending upon the length of the individual ligands and is preferably less than about 15 nm, and more preferably from about 2.9 nm to about 7 nm. For example, a tetraethylene glycol ligand has a length of about 2.9 nm, while an octaethylene glycol ligand has a length of about 4.2 nm.

Particularly preferred ligands have dopamine-based anchors and are selected from the group consisting of:

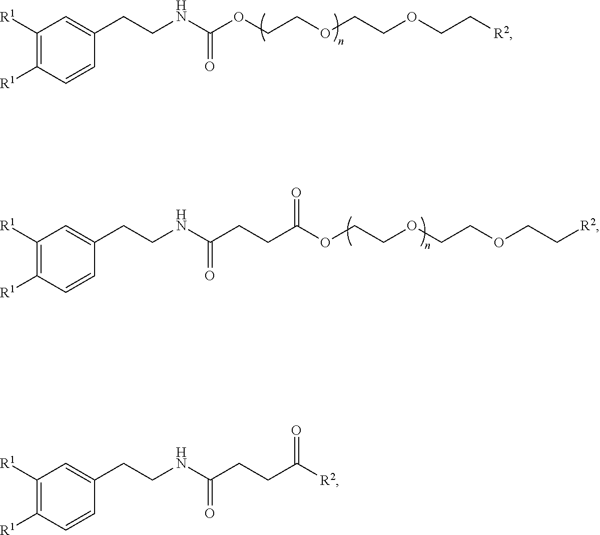

and combinations thereof, where n=2-25 (preferably 3-11), each R¹ is selected from the group consisting of protected and unprotected hydroxyl groups, each R² is individually selected from the group consisting of —OH,

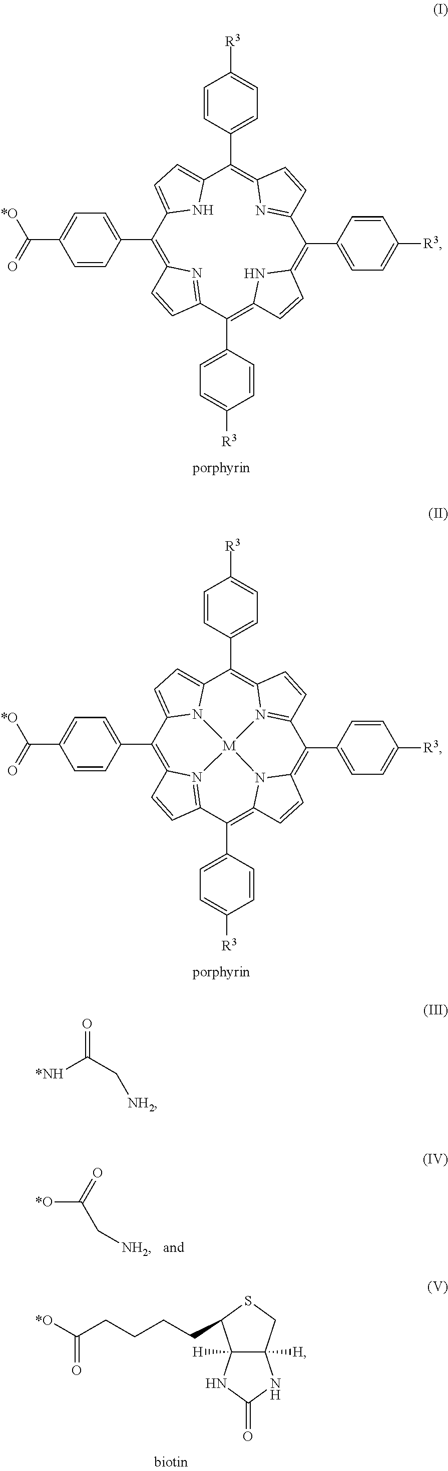

where * designates the atom where R² bonds to the ligand, each R³ is individually selected from the group consisting of —OH, —COOH, and —NH₂, —N(R⁴)₂, —N(R⁴)₃⁺, —NHR⁴, —NH—CO-AA, and —CO—NH-AA, where each R⁴ is selected from the group consisting of alkyl groups (preferably C₁-C₄ alkyl groups), AA is any amino acid, and M is selected from the group consisting of Ze, Pd²⁺, Mg²⁺, Al³⁺, Pt²⁺, Eu³⁺, and Gd³⁺. When present, preferred protecting groups are selected from the group consisting of benzyl, siloxyl, carboxylic ester, and [1,3]-dioxole (acetonide) groups. Preferably, the ligands are hydrophilic. More preferably, the ligands have an octanol/water partition coefficient (log P value) of at least about 5, and preferably from about 2 to about −1.5. The dopamine anchor aids solubility. For example, tetraethylene glycol has an octanol/water partitioning coefficient of log P=−1.26, while a dopamine-anchored tetraethylene glycol ligand has a log P of −0.2. Likewise, the log P of octaethylene glycol is −1.88, while the log P of a dopamine-anchored octaethylene glycol is −1.16.

For attachment to the oligopeptide linkages, the preferred ligands will preferably readily react with the thiol group of the terminal cysteine of the oligopeptide linkage (discussed below). The glycine on the C-terminal side will be connected via an ester bond to the alcohol function of the ligand on the other nanoparticle, forming a nanoparticle dimer.

As further discussed below, the ligands can be connected prior to attachment to the nanoparticles, or after the nanoparticles have been stealth coated. If the ligands are attached to each other before stealth coating, the protecting groups, when present, can be deprotected in one step using hydrogen/palladium on carbon.

The nanoparticle surface will preferably be essentially completely covered with ligands. That is, at least about 70%, preferably at least about 90%, and more preferably about 100% of the surface of the nanoparticle will have attached ligands. The number of ligands required to form a monolayer will be dependent upon the size of the nanoparticle (and monolayer), and can be calculated using molecular modeling or the ligand modeling methods described in Example 22. For example, a nanoparticle having a 20 nm diameter requires approximately 1.030 stealth ligands for complete surface coverage, whereas a nanoparticle with 12-nm diameter requires 412 dopamine-stealth ligands for complete surface coverage.

Various techniques for attaching ligands to the surface of various nanoparticles or to the siloxane protecting layer are known in the art. For example, nanoparticles may be mixed in a solution containing the ligands to promote the coating of the nanoparticle surface. Alternatively, coatings may be applied to nanoparticles by exposing the nanoparticles to a vapor phase of the coating material such that the coating attaches to or bonds with the nanoparticle. Preferably, the ligands attach to the nanoparticle or siloxane protecting layer through covalent bonding. Note that for dopamine-based ligand monolayers surrounding a siloxane protecting layer, both phenolic groups may not always be connected to the terminal amino-groups of the siloxane protection layer. However, the formation of one carbamate bond to the nanoparticle is sufficient for the attachment of the dopamine-based stealth ligands.

A preferred method of ligand attachment follows, where the ligands have already been linked via an oligopeptide sequence. A stoichiometric mixture (preferably about 1/1, more preferably about 10/1 per weight with respect to the mass of the nanoparticles) of the attached ligands can be reacted with the Fe/Fe₃O₄-nanoparticles in anhydrous THF. The mixture is then preferably sonicated for at least about 30 seconds and more preferably from about 1 to about 5 minutes and then continuously stirred for about 5 minutes to about 24 hours. The ligand displacement can be optionally followed up using HPLC. After completion of the stealth coating, the bimagnetic nanoparticles can be precipitated/separated with the help of a strong magnet. The particles are then preferably resuspended in THF, and recollected. Sonication for at least about 10 seconds, and preferably about 30 seconds, followed by stirring for about 5 minutes will redisperse the nanoparticles in the liquid medium. The washing/redispersion process can be repeated up to about 25 times, and preferably up to about 10 times before transferring the nanoparticles into an aqueous buffer (e.g. PBS). It will be appreciated that residual solvent can also be removed in an argon stream. Preferably, the amount of dimers (wanted) vs. monomers and oligomers is then determined using gel-permeation chromatography.

A gold coating layer can also be used to further enhance the stability of the nanoparticles and protect them from biocorrosion.

Prior to use for in vitro or in vivo experiments, the coated nanoparticles (whether or not attached) are then preferably suspended/dissolved in double-distilled and sterilized H₂O.

### Functional Groups

As shown above, in some embodiments, the nanoparticles are coated with a layer of ligands with attached functional groups for selective uptake by the target tissues. Preferred functional groups are selected from the group consisting of porphyrins, chlorins, bacteriochlorins, phthalocyanines, biotin labels, dyes, derivatives thereof, and combinations thereof.

Porphyrins (including chlorins and bacteriochlorins) have been found to trigger selective uptake by cancer cells, which over-express porphyrin receptors in their cell membranes. The LDL-receptor (low-density-lipoprotein), which is over-expressed in cancer cells, has the ability to take up porphyrins, either alone and/or by a simultaneous lipid uptake mechanism. The higher the hydrophobicity of a porphyrin, chlorin or bacteriochlorin, the easier the uptake can be facilitated by the LDL-receptor. Advantageously, this rapid uptake by cancer cells leads to the accumulation of porphyrin-doped nanoplatforms in the cancerous tissues, with only minor accumulation in other tissues such as the liver or spleen. When present, the nanoplatforms will preferably have at least about 1 attached porphyrin per nanoparticle, preferably from about 2 to about 20 attached porphyrins per nanoparticle, and even more preferably from about 5 to about 10 attached porphyrins per nanoparticle. Particularly preferred porphyrins are selected from the group consisting of metalated and unmetalated tetracarboxyphenyl porphyrins (TCPP) and tetrahydroxyphenyl porphyrins.

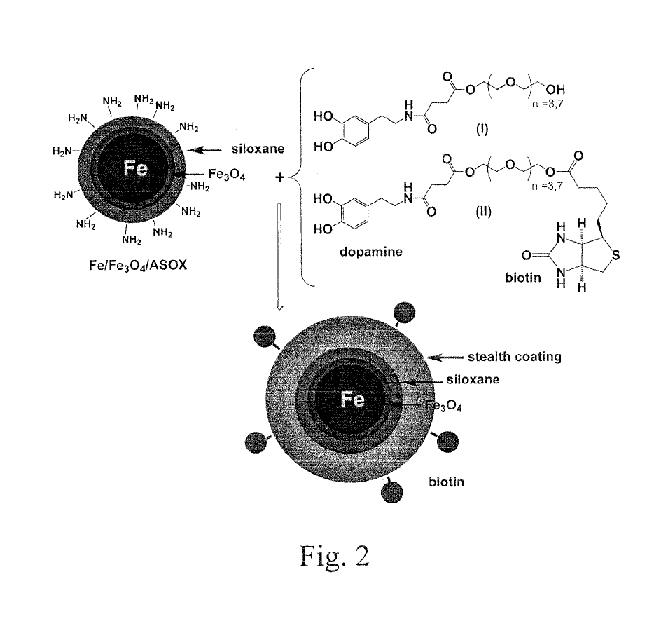

Biotin labels increase the solubility of the nanoplatforms and trigger very fast uptake processes by virtually all mammalian cells. To ensure the fastest possible uptake of the nanoplatform by the cells, as well as the highest payloads possible, the degree of biotin labeling can be varied. For that purpose, different ratios of the unlabeled and biotin-labeled ligands can be mixed with the nanoparticles. See for example, the scheme in FIG. 2 which shows the biotin labeling of the preferred Fe/Fe₃O₄ nanoparticles. Preferably the unlabeled to labeled ligands are mixed at a ratio of about 1:1 to about 200:1. Because of their similar steric demands, the ligands are most likely to follow a statistical distribution between the Fe/Fe₃O₄/ASOX nanoparticles that can be described by the Poisson distribution (see Example 24). As a consequence, the number of biotinylated organic ligands per nanoparticle will vary, although the distribution will preferably be relatively narrow: for more than 95% of the nanoparticles, the maximal deviation from each other will preferably be less than 10 relative percent. Furthermore, there will be a kinetic selection process during cell loading, because the nanoplatforms featuring the optimal structure will be taken up first. When present, the nanoplatforms will preferably have at least about 1 biotin label, preferably from about 1 to about 50 biotin labels per nanoparticle, and even more preferably from about 2 to about 10 biotin labels per nanoparticle.

### Oligopeptide Linkages and Consensus Sequences

Suitable oligopeptide linkages will comprise the consensus sequence for the target protease, a terminal carboxylic acid group (C terminus), and a terminal amine group (N terminus). The oligopeptide can also preferably comprise a thiol group at the C terminus, and a carboxylic acid group at the N terminus. In some embodiments, the oligopeptide linker comprises a hydrophilic region of at least 10 amino acids N-terminal to the protease consensus sequence, and a linking region C-terminal to the cleavage sequence, wherein the C-terminal linking region comprises a thiol reactive group at its terminus. Even more preferably, the C terminus of the oligopeptide comprises a cysteine residue, lysine, or aspartate. The N-terminal hydrophilic region of the oligopeptide preferably has an excess positive or negative charge at a ratio of about 1:1. The N-terminal hydrophilic region also preferably comprises amino acid residues capable of forming hydrogen bonds with each other.

Particularly preferred C-terminal linking regions comprise a sequence selected from the group consisting of GGGC (SEQ ID NO: 14), AAAC (SEQ ID NO: 15), SSSC (SEQ ID NO: 16), TTTC (SEQ ID NO: 17), GGC (SEQ ID NO: 38), GGK (SEQ ID NO: 39), GC (SEQ ID NO: 40), GGD (SEQ ID NO: 42), GXGD (SEQ ID NO: 58), and GXGXGD (SEQ ID NO: 59), where X is any amino acid other than cysteine or lysine. Particularly preferred N-terminal regions of the oligopeptide comprise a sequence selected from the group consisting of SRSRSRSRSR (SEQ ID NO: 1), KSRSRSRSRSR (SEQ ID NO: 19), KKSRSRSRSRSR (SEQ ID NO: 20), CGGG (SEQ ID NO: 23), KGGG (SEQ ID NO: 24), KGG (SEQ ID NO: 37), KGXG (SEQ ID NO: 60), and KGXGXG (SEQ ID NO: 61), where X is any amino acid other than cysteine or lysine, and DGXG (SEQ ID NO: 62) and DGXGXG (SEQ ID NO: 63), where X is any amino acid other than cysteine. The N-terminus can also comprise one or more terminal groups selected from the group consisting of lysine, ornithine, 2,4 diaminobutyric acid, and 2,3 diaminoproprionic acid. Another preferred oligopeptide has the following general structure:

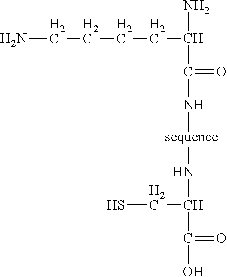

where the “sequence” can be any of the oligopeptide or consensus sequences described herein. The oligopeptides can be purchased, or they can be synthesized using known methods (e.g., modified Merrifield synthesis).

Preferably, the consensus sequence used in the oligopeptide linkages is selected from the group consisting of serine protease cleavage sequences, aspartyl protease cleavage sequences, cysteine protease cleavage sequences, and metalloprotease cleavage sequences. Even more preferably, the consensus sequence comprises a cleavage sequence for a protease selected from the group consisting of urokinase, matrix metallopeptidase, cathepsin, and gelatinase. Particularly preferred proteases and their corresponding consensus sequences are listed in Table I below.

TABLE I

| Protease | Consensus Sequence (Cleavage Sequence) |
|---|---|
| MMP-1 | VPMSMRGG (SEQ ID NO: 3 and variants thereof which may be deleted |
| | at the C-terminus by 1 residue) |
| MMP-2 | IPVSLRSG (SEQ ID NO: 4) |
| MMP-3 | RPFSMIMG (SEQ ID NO: 5) |
| MMP-7 | VPLSLTMG (SEQ ID NO: 6) |
| MMP-9 | VPLSLYSG (SEQ ID NO: 7) |
| MMP-11 | HGPEGLRVGFYESDVMGRGHARLVHVEEPHT (SEQ ID NO: 25) |
| | GAANLVRG (SEQ ID NO: 74) |
| MMP-13 | GPQGLAGQRGIV (SEQ ID NO: 26) |
| MMP-14 | IPESLRAG (SEQ ID NO: 8) |
| uPA | SGRSA (SEQ ID NO: 2) |
| Cathepsin B | SLLKSRMVPNFN (SEQ ID NO: 27) |
| | DAFK (SEQ ID NO: 10) |
| Cathepsin D | SLLIFRSWANFN (SEQ ID NO: 28) |
| | SGKPILFFRL (SEQ ID NO: 11) |
| Cathepsin E | SGSPAFLAKNR (SEQ ID NO: 9) |
| | SGKPIIFFRL (SEQ ID NO: 12) |
| Cathepsin K | PRAGA (SEQ ID NO: 75) |
| Cathepsin L | SGVVIATVIVIT (SEQ ID NO: 29) |
| Gelatinase | GPLGMISQ (SEQ ID NO: 13) |

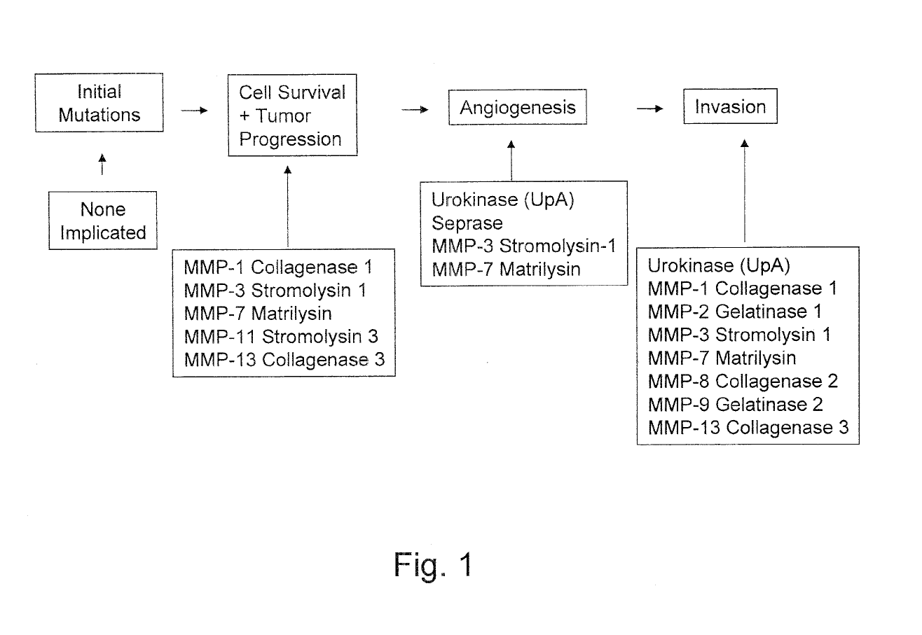

With reference to FIG. 1, the foregoing proteases are associated with many specific events in cancer progression. The stages of disease progression are separated into four events: initial mutation, cell survival/tumor progression, angiogenesis (development of new blood vessels), and invasion/tissue remodeling. The array of proteases associated with each stage can give a good picture of how far the cancer has progressed and what the prognosis will be. The most preferred oligopeptide sequences for select proteases are listed in the table below with the point of cleavage indicated by “−”.

TABLE II

| Protease | Preferred Oligopeptide with Consensus Sequence |
|---|---|
| MMP-1 | KGGVPMS-MRGGGC (SEQ ID NO: 30) |
| | HHHGAGVPMS-MRGAG (SEQ ID NO: 76)* |
| MMP-2 | KGGIPVS-LRSGGC (SEQ ID NO: 31) |
| | HHHGAGIPVS-LRSGAG (SEQ ID NO: 77)* |
| MMP-3 | HHHGAGRPFS-MIMGAG (SEQ ID NO: 78)* |
| MMP-7 | KGGVPLS-LTMGGC (SEQ ID NO: 32) |
| | HHHGAGVPLS-LTMGAG (SEQ ID NO: 79)* |
| MMP-9 | HHHGAGVPLS-LYSGAG (SEQ ID NO: 80)* |
| MMP-11 | HHHGAGGAAN-LVRGGAG (SEQ ID NO: 81)* |
| MMP-13 | HHHGAGPQGLA-GQRGIVGAG (SEQ ID NO: 82)* |
| uPA | KGGGSGR-SAGGGC (SEQ ID NO: 33) |
| | CGGGSGR-SAGGC (SEQ ID NO: 34) |
| | CGGGSGR-SAGGGC (SEQ ID NO: 35) |
| | DGGSGR-SAGGK (SEQ ID NO: 36) |
| | SRSRSRSRSRSGR-SAGGGC (SEQ ID NO: 18) |
| | KGGSGR-SAGGD (SEQ ID NO: 41) |
| | CGGGSGR-SAGGG (SEQ ID NO: 64) |
| | DGGGSGR-SAGGGD (SEQ ID NO: 65) |
| | DGAGSGR-SAGAGD (SEQ ID NO: 66 and variants thereof, which may be |
| | deleted at the N-terminus by 1 residue and C-terminus by 1 or 2 residues) |
| | KGGSGR-SAGGG (SEQ ID NO: 67) |
| | DGGSGR-SAGGGC (SEQ ID NO: 68) |
| | HHHGAGSGR-SAGAG (SEQ ID NO: 83)* |
| Cathepsin B | HHHGAGSLLKSR-MVPNFNGAG (SEQ ID NO: 84)* |
| Cathepsin D | HHHGAGSLLIFR-SWANFNGAG (SEQ ID NO: 85)* |
| Cathepsin L | HHHGAGSGVVIA-TVIVITGAG (SEQ ID NO: 86)* |
| Cathepsin K | HHHGAGPR-AGAG (SEQ ID NO: 87)* |

*(including variants thereof, which may be deleted at the N-terminus by 1, 2, or 3 residues)

With reference again to FIG. 1, an accurate cancer prognosis can be determined using the inventive assays. In particular, if MMP-1 and MMP-7, but neither of the other two proteases are detected by the inventive assays, the cancer prognosis is for cell survival/tumor progression. If uPA and MMP-7 are detected by the assays (but not MMP-1 or MMP-2), the prognosis is for angiogenesis. If all four proteases are detected, the prognosis is for invasion and eventual metastasis. Thus, the in-vivo measurements of these four proteases enable the spatially resolved determination of the progression of cancerous tissue, and permit a more detailed prognosis that can guide the treatment towards the most active tumors in the body,

In the presence of the protease, the consensus sequence of the nanoplatform assembly is cleaved, and the change caused by this cleavage is detected by the inventive MRI and light backscattering assays. Thus, depending upon the proteases targeted by the nanoplatform, two or more of the following sequences will result: KGGVPMS (SEQ ID NO: 43), MRGGGC (SEQ ID NO: 44), KGGIPVS (SEQ ID NO: 45), LRSGGC (SEQ ID NO: 46), KGGVPLS (SEQ ID NO: 47), LTMGGC (SEQ ID NO: 48), KGGGSGR (SEQ ID NO: 49), SAGGGC (SEQ ID NO: 50), CGGGSGR (SEQ ID NO: 51), SAGGC (SEQ ID NO: 52), DGGSGR (SEQ ID NO: 53), SAGGK (SEQ ID NO: 54), SRSRSRSRSRSGR (SEQ ID NO: 55), KGGSGR (SEQ ID NO: 56), SAGGD (SEQ ID NO: 57), SAGGG (SEQ ID NO: 69), DGGGSGR (SEQ ID NO: 70), SAGGGD (SEQ ID NO: 71), DGAGSGR (SEQ ID NO: 72) (and variants thereof which may be deleted at the N-terminus by 1 residue), SAGAGD (SEQ ID NO: 73) (and variants thereof which may be deleted at the C-terminus by 1 residue), HHHGAGVPMS (SEQ ID NO: 88)*, MRGAG (SEQ ID NO: 89), HHHGAGIPVS (SEQ ID NO: 90)*, LRSGAG (SEQ ID NO: 91), HHHGAGSGR (SEQ ID NO: 92)*, HHHGAGRPFS (SEQ ID NO: 93)*, MIMGAG (SEQ ID NO: 94), HHHGAGVPLS (SEQ ID NO: 95)*, LTMGAG (SEQ ID NO: 96), HHHGAGVPLS (SEQ ID NO: 97)*, LYSGAG (SEQ ID NO: 98), HHHGAGGAAN (SEQ ID NO: 99)*, LVRGGAG (SEQ ID NO: 100), HHHGAGPQGLA (SEQ ID NO: 101)*, GQRGIVGAG (SEQ ID NO: 102), HHHGAGSLLKSR (SEQ ID NO: 103)*, MVPNFNGAG (SEQ ID NO: 104), HHHGAGSLLIFR (SEQ ID NO: 105)*, SWANFNGAG (SEQ ID NO: 106), HHHGAGSGVVIA (SEQ ID NO: 107)*, TVIVITGAG (SEQ ID NO: 108), HHHGAGPR (SEQ ID NO: 109)*, or AGAG (SEQ ID NO: 110), where * indicates included sequence variants where the sequence may be deleted by 1, 2, or 3 residues at the N-terminus.

### Nanoplatform Structures

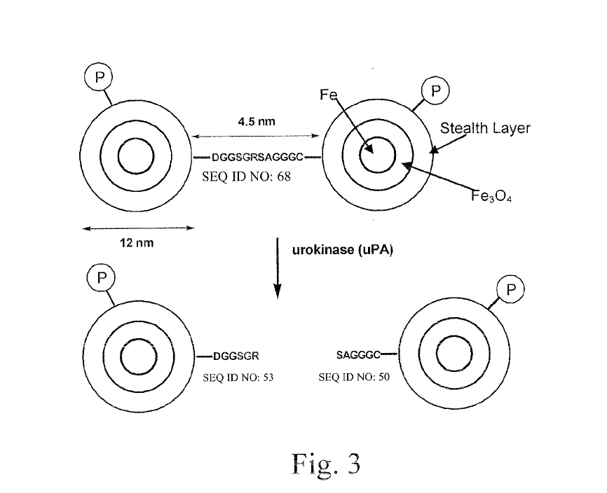

Linked nanoplatforms will preferably be used for protease detection (e.g., MRI contrast agents or light backscattering). The diagnostic nanoplatforms can be linked in various ways. In one embodiment, the nanoplatform assemblies will comprise at least two nanoplatforms linked together via one or more oligopeptide linkages. As previously noted, the oligopeptide linkages can be linked directly to the nanoparticles of the respective nanoplatforms, or to the one or more monolayers surrounding the nanoparticle. The nanoparticles may feature chemically attached functional groups, such as porphyrins or biotin labels. Such functional groups may be bound directly to the nanoparticle or protective layer, or they may be bound to the nanoparticle (with or without a monolayer) via an oligopeptide linkage. FIG. 3 illustrates (not to scale) two nanoplatforms comprising superparamagnetic Fe/Fe₃O₄-nanoparticles linked by an oligopeptide linkage comprising a consensus sequence for urokinase. ‘P’ stands for porphyrin (such as tetra-4-carboxyphenyl porphyrin, TCPP), which is linked to the stealth-coating of the Fe/Fe₃O₄-nanoparticles.

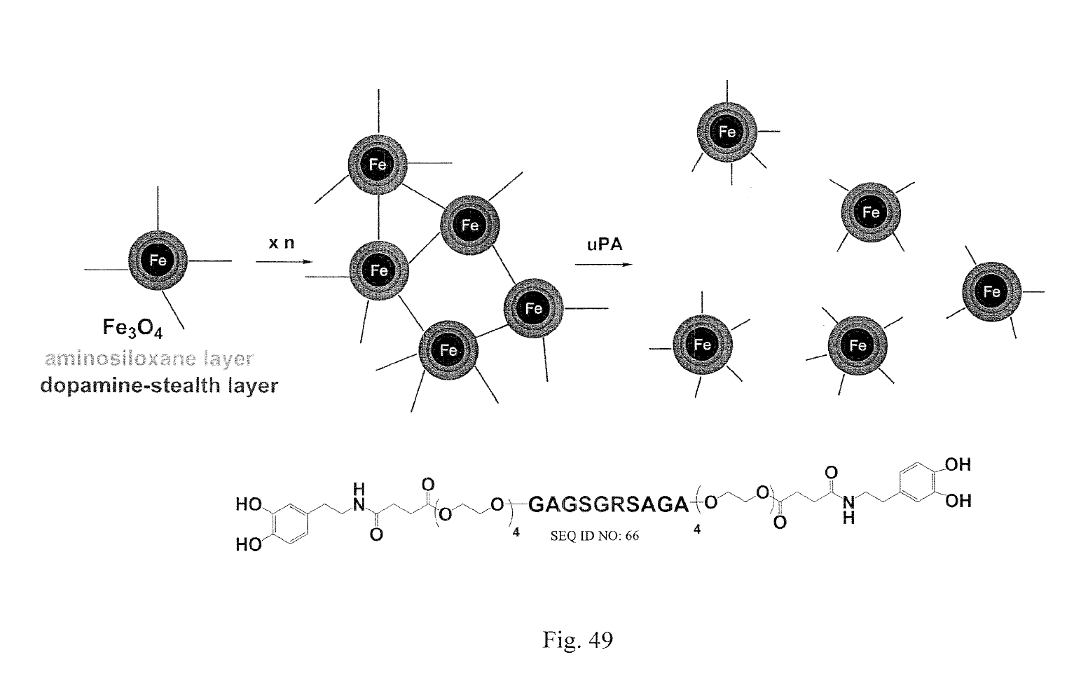

In some embodiments, multiple nanoparticles can be bound to a central structure via one or more oligopeptide linkages. Suitable central structures are selected from the group consisting of nanoparticles and porphyrins. FIG. 4 depicts the linkage of two nanoplatforms utilizing a porphyrin central structure featuring four cleavage sequences bound to the stealth-coating of the nanoparticles. Multiple nanoplatforms can also be linked together to form oligomeric complexes, as shown in FIG. 49. These nanoplatform or nanoparticle oligomers can further comprise particles other than nanoparticles (described below) as part of the oligomeric matrix. The nanoplatforms can also be functionalized as discussed herein.

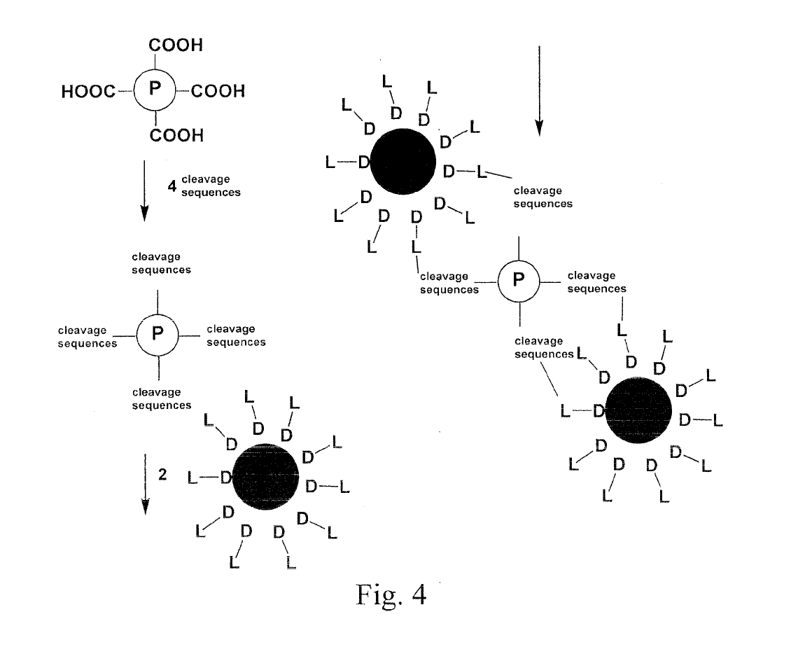

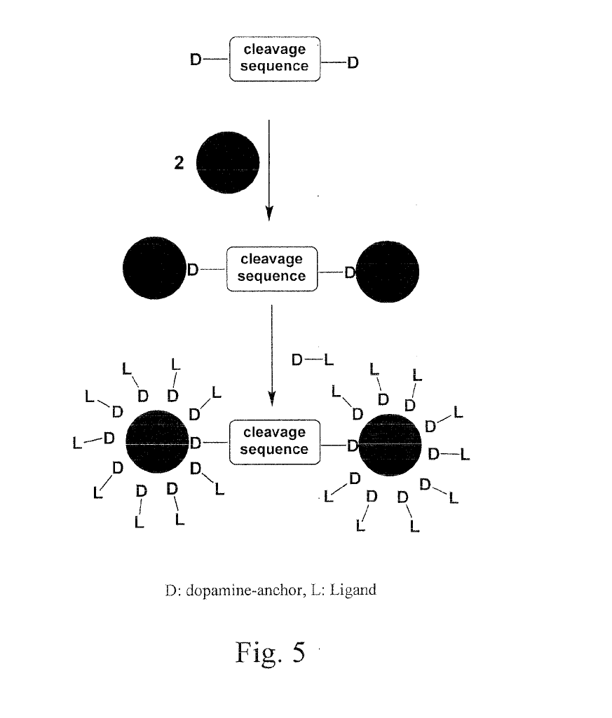

It will be appreciated that the various components of the theranostic platforms can be assembled in different orders. For example, the nanoparticles can be stealth coated, and then linked via the oligopeptide sequence. Likewise, the ligands can first be linked via an oligopeptide comprising the target cleavage sequence and then attached to the nanoparticles. FIG. 5 illustrates this process. The porphyrin can be attached to the ligand layer before or after coating. Regardless, the distance between the linked nanoplatforms is preferably from about 5 nm to about 70 nm, and more preferably from about 10 nm to about 30 ram.

The nanoplatforms for therapeutic treatment of cancerous tissues will preferably be unlinked. These nanodevices will preferably comprise a core/shell nanoparticle and a stealth ligand coating. In some embodiments, the nanoplatforms will also preferably include a siloxane protecting layer. Even more preferably, the nanoplatforms will feature chemically attached functional groups, such as porphyrins, biotin labels, and combinations thereof. Again, the components of the nanoplatforms can be assembled in various orders. The therapeutic nanoplatforms are particularly suited for hyperthermia treatment of cancerous tissues.

Regardless of the detection or treatment method, for in vivo use, the nanoplatforms preferably biocorrode after about 2 days to about 5 days, and are cleared from the patient's systems after about 10 days. More preferably, the nanoplatforms comprising siloxane protective layers will biocorrode after about 5 days to about 15 days, and are cleared from the patient's systems after about 30 days. Conversely, the nanoplatforms will preferably remain in vivo without biocorroding for at least a period of 2 days after administration.

Moreover, when used in vivo, the nanoplatforms preferably do not coagulate, but remain as distinct individual or linked nanostructures. In addition, when used in vivo, the majority of the administered nanoplatforms will preferably be taken up and localize in the cancerous tissue. That is, only small amounts of the nanoplatforms will be found in healthy tissues, such as the liver or spleen. For example, when 150 μg of nanoplatforms are administered by IV injection, at least about 50% of the total administered nanoplatforms preferably localize in the target tissue (tumor), while less than about 10% of the nanoplatforms preferably localize in healthy tissues. When 500 μg of nanoplatforms are administered (2 consecutive IV-injections of 250 μg each within 24 hours), at least about 30% to about 50% of the total administered nanoplatforms localize in the target tissue (tumor).

### Particles

In some embodiments, a nanoplatform will be linked to a particle (instead of a second nanoplatform, as described above). For example, the ligand protective layer of the nanoplatform can be linked via an oligopeptide linkage (e.g., SEQ ID NO: 66 variant) to a particle, such as TCPP, shown below.

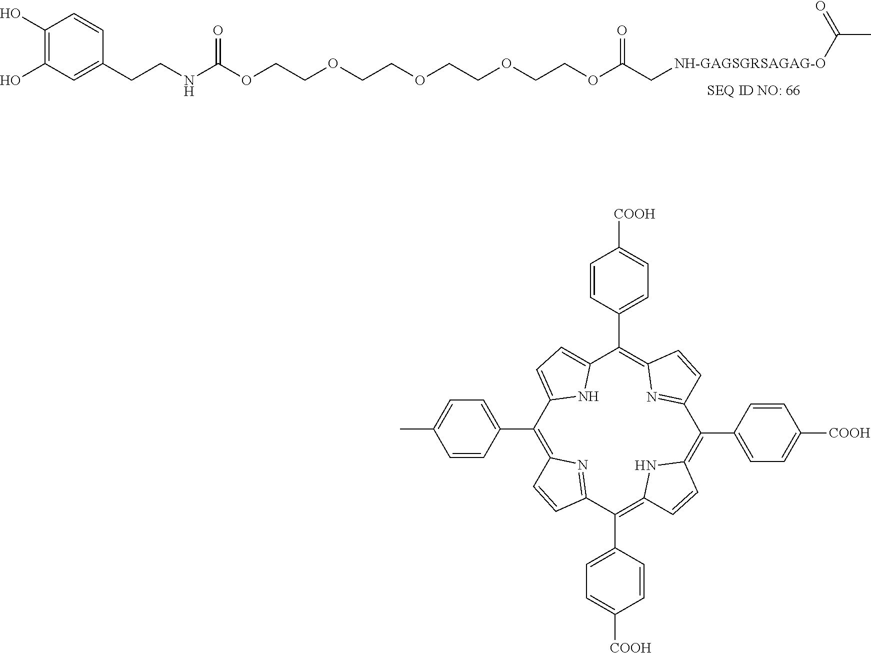

These embodiments are particularly useful for assays and methods of monitoring the progress of cancer treatment in a mammal. A number of different types of particles can be used to form these nanoplatform assemblies, depending upon the type of sensor used to measure the protease activity, as discussed in more detail below. Preferably, the excitation and emission spectral maxima of the particles are between 650 and 800 nm. Preferred particles for use in the diagnostic assays are selected from the group consisting of chromophores/luminophores (dyes), quantum dots, viologens, and combinations thereof.

### 1. Chromophores/Luminophores

Chromophore/luminophore particles suitable for use in the inventive assays include any organic or inorganic dyes, fluorophores, phosphosphores, light absorbing nanoparticles (e.g., Au, Ag, Pt, Pd), combinations thereof, or the metalated complexes thereof. Preferably, the chromophore/luminophore particles have a size of less than about 100 nm.

Suitable organic dyes are selected from the group consisting of coumarins, pyrene, cyanines, benzenes, N-methylcarbazole, erythrosin B, N-acetyl-L-tryptophanamide, 2,5-diphenyloxazole, rubrene, and N-(3-sulfopropyl)acridinium. Specific examples of preferred coumarins include 7-aminocoumarin, 7-dialkylamino coumarin, and coumarin 153. Examples of preferred benzenes include 1,4-bis(5-phenyloxazol-2-yl)benzene and 1,4-diphenylbenzene. Examples of preferred cyanines include oxacyanines, thiacyanines, indocyanins, merocyanines, and carbocyanines. Other exemplary cyanines include ECL Plus, ECF, C3-Oxacyanine, C3-Thiacyanine Dye (EtOH), C3-Thiacyanine Dye (PrOH), C5-Indocyanine, C5-Oxacyanine, C5-Thiacyanine, C7-Indocyanine, C7-Oxacyanine, CypHer5, Dye-33, Cy7, Cy7.5, Cy5.0, Cy5.5, Cy3Cy5 ET, Cy3B, Cy3.0, Cy3.5, Cy2, CBQCA, NIR1, NIR2, NIR3, NIR4, NIR820, SNIR1, SNIR2, SNIR4, Merocyanine 540, Pinacyanol-Iodide, 1,1-Diethyl-4,4-carbocyanine iodide, Stains All, Dye-1041, or Dye-304.

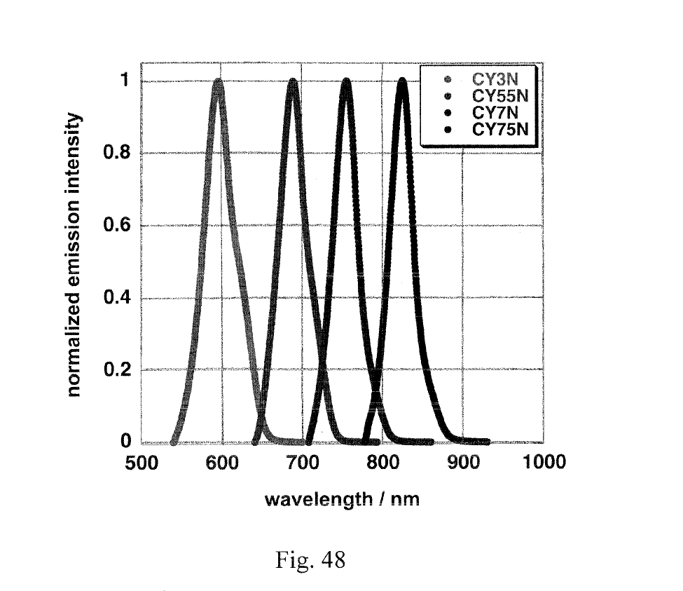

Cyanine dyes are particularly preferred organic dyes for use in the nanoplatforms. The fluorescent cyanine dye is tethered to the nanoparticle and experiences rapid fluorescence quenching by the plasmon of the Fe(0)-core. This is observed as long as the tether is smaller than the Förster-radius of the cyanine dye (5-6 nm for Cy3.0 and Cy3.5, 6-7 nm for Cy5.0 and Cy5.5, and approx. 7 nm for Cy7 and Cy7.5). The maximal length of the tether, consisting of the ligand (˜2.84 nm) and not more than 12 amino acid residues in the cleavage sequences (up to 4 nm) indicates that shorter cleavage sequences (uPA and MMP's) are suitable for use with Cy3.x and Cy5.x dyes, whereas the cathepsins are preferably linked to Cy5.x and Cy.7.x dyes to permit optimal quenching of the tethered cyanine dyes. For all of the cyanines, their emission maxima are red-shifted with respect to the autofluorescence of human urine. Multiple cyanines can be linked to a single nanoparticle to create oligoplexing nanoplatforms, as shown in FIG. 47, to measure the activity of up to four enzymes simultaneously. All four dyes in the UVA or blue region of the electromagnetic spectrum can be excited simultaneously, or each dye can be excited individually. All cyanine dyes have an excitation maximum, which is blueshifted by 20-25 μm with respect to their emission maximum (typical for fluorescent singlet states). The emission spectra of NS-Cy3.0 (λex=538, λem=560), NS-Cy5.5 (λex=639, λem=660), NS-Cy7.0 (λex=740, λem=760) and NS-Cy7.5 (λex=808, λem=830) are shown in FIG. 48. Suitable inorganic dyes are selected from the group consisting of metalated and non-metalated porphyrins, phthalocyanines, chlorins (e.g., chlorophyll A and B), and metalated chromophores. Preferred porphyrins are selected from the group consisting of tetra carboxy-phenyl-porphyrin (TCPP) and Zn-TCPP. Preferred metalated chromophores are selected from the group consisting of ruthenium polypyridyl complexes, osmium polypyridyl complexes, rhodium polypyridyl complexes, 3-(1-methylbenzoimidazol-2-yl)-7-(diethylamine)-coumarin complexes of iridium(III), and 3-(benzothiazol-2-yl)-7-(diethylamino)-coumarin complexes with iridium(III).

Suitable fluorophores and phosphosphores are selected from the group consisting of phosphorescent dyes, fluoresceines, rhodamines (e.g., rhodamine B, rhodamine 6G), and anthracenes (e.g., 9-cyanoanthracene, 9,10-diphenylanthracene, 1-Chloro-9,10-bis(phenyl-ethynyl)anthracene).

### 2. Quantum Dots

A quantum dot is a semiconductor composed of atoms from groups II-VI or III-V elements of the periodic table (e.g., CdSe, CdTe, InP). The optical properties of quantum dots can be manipulated by synthesizing a (usually stabilizing) shell. Such quantum dots are known as core-shell quantum dots (e.g., CdSe/ZnS, InP/ZnS, InP/CdSe). Quantum dots of the same material, but with different sizes, can emit light of different colors. Their brightness is attributed to the quantization of energy levels due to confinement of an electron in all three spatial dimensions. In a bulk semiconductor, an electron-hole pair is bound within the Bohr exciton radius, which is characteristic for each type of semiconductor. A quantum dot is smaller than the Bohr exciton radius, which causes the appearance of discrete energy levels. The band gap, ΔE, between the valance and conduction band of the semiconductor is a function of the nanocrystal's size and shape. Quantum dots feature slightly lower luminescence quantum yields than traditional organic fluorophores but they have much larger absorption cross-sections and very low rates of photobleaching. Molar extinction coefficients of quantum dots are about 10⁵-10⁶ M⁻¹ cm⁻¹, which is 10-100 times larger than dyes.

Core/shell quantum dots have higher band gap shells around their lower band gap cores, which emit light without any absorption by the shell. The shell passivates surface nonradiative emission from the core thereby enhancing the photoluminescence quantum yield and preventing natural degradation. The shell of type I quantum dots (e.g. CdSe/ZnS) has a higher energy conduction band and a lower energy valance band than that of the core, resulting in confinement of both electron and hole in the core. The conduction and valance bands of the shell of type II quantum dots (e.g., CdTe/CdSe, CdSe/ZnTe) are either both lower or both higher in energy than those of the core. Thus, the motions of the electron and the hole are restricted to one dimension. Radiative recombination of the exciton at the core-shell interface gives rise to the type-II emission. Type II quantum dots behave as indirect semiconductors near band edges and therefore, have an absorption tail into the red and near infrared. Alloyed semiconductor quantum dots (CdSeTe) can also be used, although types I and II are most preferred. The alloy composition and internal structure, which can be varied, permits tuning the optical properties without changing the particles' size. These quantum dots can be used to develop near infrared fluorescent probes for in vivo biological assays as they can emit up to 850 nm.

Particularly preferred quantum dots are selected from the group consisting of CdSe/ZnS core/shell quantum dots, CdTe/CdSe core/shell quantum dots, CdSe/ZnTe core/shell quantum dots, and alloyed semiconductor quantum dots (e.g., CdSeTe). The quantum dots are preferably small enough to be discharged via the renal pathway when used in vivo. More preferably, the quantum dots are less than about 10 nm in diameter, even more preferably from about 2 nm to about 5.5 nm in diameter, and most preferably from about 1.5 nm to about 4.5 nm in diameter. If different color emission is needed for creating multiple sensors (multiplex detection), this can be achieved by changing the size of the quantum dot core yielding different emission wavelengths. The quantum dots can be stabilized or unstabilized as discussed above regarding nanoparticles. Preferred ligands for stabilizing quantum dots are resorcinarenes.

### Cell Delivery

In some embodiments, the nanoplatforms and assemblies can be loaded into cells for targeted delivery of the cells to cancerous tissue. For each of the methods discussed herein, in vivo delivery to the cancerous tissue may be accomplished using cellular delivery. Cellular delivery is a particularly preferred delivery method for magnetic hyperthermia treatment, discussed herein. Suitable cells for delivering the nanoplatforms to the cancerous tissues include any tumor-tropic cells. Preferred cells include stem cells, monocytes, macrophages, and combinations thereof. Stem cells particularly suited for selective delivery to cancerous tissue include neural stem cells (NSCs), umbilical cord matrix stem cells, bone marrow stem cells, and adipose derived mesenchymal stem cells. In one embodiment, the cells are loaded with iron/iron oxide nanoplatforms and assemblies by incubating the cells in a suitable culture medium (such as fetal bovine serum (FBS)) containing the nanoplatforms and assemblies at a level providing a total Fe concentration of from about 1 mg/l to about 250 mg/l (and preferably from about 10 mg/l to about 100 mg/l) for about 1 to about 72 hours (and preferably for about 12 to about 24 hours). Preferably, the amount of Fe loaded into each cell is from about 0.1 pg (picogram) per cell to about 10 pg/cell (and more preferably from about 1 pg/cell to about 5 pg/cell).

### The Inventive Methods

One advantage of the inventive nanoplatforms is the flexibility to adapt the nanodevices and assays by modifying the nanoparticles, particles, protective layers, or functional groups to suit the sensor technology available, and likewise, using a variety of sensor technologies for detecting enzyme activity in cancerous tissues. Advantageously, the same nanoplatforms can also be used for targeted therapeutic treatment of the cancerous tissue.

The nanoplatforms can be used to detect cancerous or pre-cancerous cells associated with a cancer selected from the group consisting of an AIDS-related cancer, AIDS-related lymphoma, anal cancer, appendix cancer, childhood cerebellar astrocytoma, childhood cerebral astrocytoma, basal cell carcinoma, extrahepatic bile duct cancer, childhood brain stem glioma, adult brain tumor, childhood malignant glioma, childhood ependymoma, childhood medulloblastoma, childhood supratentorial primitive neuroectodermal tumors, childhood visual pathway and hypothalamic glioma, breast cancer, pregnancy-related breast cancer, childhood breast cancer, male breast cancer, childhood carcinoid tumor, gastrointestinal carcinoid tumor, primary central nervous system lymphoma, cervical cancer, colon cancer, childhood colorectal cancer, esophageal cancer, childhood esophageal cancer, intraocular melanoma, retinoblastoma, adult glioma, adult (primary) hepatocellular cancer, childhood (primary) hepatocellular cancer, adult Hodgkin lymphoma, childhood Hodgkin lymphoma, islet cell tumors, Kaposi Sarcoma, kidney (renal cell) cancer, childhood kidney cancer, adult (primary) liver cancer, childhood (primary) liver cancer, Non-small cell liver cancer, small cell liver cancer, AIDS-related lymphoma, Burkitt lymphoma, adult Non-Hodgkin lymphoma, childhood Non-Hodgkin lymphoma, primary central nervous system lymphoma, melanoma, adult malignant mesothelioma, childhood mesothelioma, metastatic squamous neck cancer with occult primary, mouth cancer, childhood multiple endocrine neoplasia syndrome, multiple myeloma/plasma cell neoplasm, mycosis fungoides, myelodysplastic syndromes, myelodysplastic/myeloproliferative diseases, adult acute myeloid leukemia, childhood acute myeloid leukemia, multiple myeloma, neuroblastoma, non-small cell lung cancer, childhood ovarian cancer, ovarian epithelial cancer, ovarian germ cell tumor, ovarian low malignant potential tumor, pancreatic cancer, childhood pancreatic cancer, islet cell pancreatic cancer, parathyroid cancer, penile cancer, plasma cell neoplasm/multiple myeloma, pleuropulmonary blastoma, prostate cancer, rectal cancer, childhood renal cell cancer, renal pelvis and ureter, transitional cell cancer, adult soft tissue sarcoma, childhood soft tissue sarcoma, uterine sarcoma, skin cancer (nonmelanoma), childhood skin cancer, melanoma, Merkel cell skin carcinoma, small cell lung cancer, small intestine cancer, squamous cell carcinoma, stomach cancer, childhood stomach cancer, cutaneous T-Cell lymphoma, testicular cancer, thyroid cancer, childhood thyroid cancer, and vaginal cancer.

The assemblies can also be used to monitor the progression of cancer treatment in a mammal.

For each of the in vivo methods discussed below, the nanoplatforms can be administered using any suitable method, including without limitation, intravenously, subcutaneously, or via localized injection directly into or near the tumor site (i.e., intratumoral or peritumoral). These administration routes are also suitable for use in conjunction with liposomal or cellular delivery methods discussed herein.

### Detection and Imaging

### 1. Magnetic Resonance Imaging

In one aspect of the invention, the inventive nanoplatforms work on the basis of spin-relaxation times of protons (¹H) in tissues or biological samples. The diagnostic nanoplatforms work as MRI contrast agents, which alter the T₁ and/or T₂ relaxation times of the ¹H nuclei in the tissue or sample. For in vivo imaging, this changes the signal intensity of the tissue being imaged. The linked nanoplatform assay, or composition comprising the linked nanoplatforms, is preferably administered to a mammal using a pharmaceutically-acceptable carrier. The nanoplatform can be administered by intravenous (IV) injection into the bloodstream. Preferably, about 200 μg of linked nanoplatforms are administered by IV-injection. Alternatively, the linked nanoplatforms dissolved in an aqueous buffer (e.g., phosphate buffered saline (PBS)) can be administered by injection to a localized region, such as directly into or near the tumor site. Liposomal delivery may also be used, including thermolabile liposomes. Cellular delivery can also be used.

MRI data acquisition can start almost immediately after injection. MRI data acquisition preferably begins once the nanoplatform contrast agents have been taken up by the cancerous cells and localize in the target area of the body or sample. The concentration of the nanoplatform assay in the target tissue is preferably from about 1 μg/g of tissue to about 1,000 μg/g of tissue, and more preferably from about 10 μg/g of tissue to about 250 μg/g of tissue. Meaningful data is preferably acquired after about 15 minutes to about 24 hours after injection of the linked nanoplatform assays, and more preferably after about 30 min. to about 5 hours, depending upon when data acquisition begins. Short RF pulses are transmitted into the region or sample of interest. The pulse sequences can be modified depending upon whether the tissue contrast will be determined mainly by differences in T₁ (T₁-weighted image) or T₂ (T₂-weighted image). Automatic data collection and analysis can be implemented using a computer program (i.e., algorithm) for assessing, preferably in real time, the data transmitted or collected from the MRI machine. The pulse sequence parameters can be further adjusted by the machine operator to maximize contrast.

A preferred sequence for use in the inventive method is a Carr-Purcell Meiboom-Gill spin-echo sequence. This sequence uses a 90° excitation pulse followed by an echo train induced by a series of 180° refocusing pulses separated by an array of times set by the user to achieve full decay of the signal. Data is acquired during the spin echo. CPMG spin-echo sequences produce T₂-weighted images. The pulse sequence and MR data acquisition process can be repeated as many times as necessary to collect multiple sets of data over a given period of time until the nanoplatforms begin to biocorrode (at least about 2 days, and preferably from about 5-15 days when a siloxane protective layer is used). It will be appreciated that the total number and frequency of the repetitive MRI scans depends upon the instrumentation used. Advantageously, the results can be read within about 1 hour after administration of the nanoplatforms. These data sets can then be compared to determine any changes. In the presence of the target protease, the oligopeptide linkage between the nanoplatforms is cleaved, separating the nanoplatforms. As a consequence, a dramatic change in T₂ will preferably be observed in the MRI data over time. In general, the greater the observed change in T₂, the more active the cancerous tissue. Preferably, a change in T₂ of greater than about a factor of 5 (preferably from about 5 to about 10) is correlated to a developing cancer, and more preferably, a change in T₂ of greater than about a factor of 10 is correlated to an active (metastatic) cancer. It is particularly preferred that the observed T₁ values remain substantially unchanged.

The inventive MRI contrast agents preferably have relaxivities of r₁ of greater than about 100 mM⁻¹s⁻¹ for T₁-enhancement and an r₂ with an integer number greater than about −2,000 mM⁻¹s⁻¹ (that is −3,000 mM⁻¹ s⁻¹ is considered to be greater than −2,000 mM⁻¹s⁻) for T₂-decrease.

Strong T₁-weighting can be achieved by using an inversion recovery pulse. In this sequence, the acquisition sequences is preceded by a 180° RF pulse, which inverts the longitudinal magnetization. The signal is then acquired during recovering of the longitudinal magnetization towards equilibrium. The interval between the inversion pulse and the first acquisition sequence is called the inversion time, T1. The rate of recovery is inversely proportional to T₁.

The acquired data can then be used to generate an image. More specifically, depending on the pulse sequence used, a computer utilizes a software program to construct the image based upon the data. Suitable MR apparatuses and programs are known in the art. It will be appreciated that the change in T₁ or T₂ caused by the cleavage of the protease sequence is visually discernable as increased contrast and changes in the images over time. For example, data acquisition can be set up to make large T₂ times brighter in the generated image, or short T₂ times can be set up to give a brighter image. In general, it is preferred that the stronger signal be correlated with a brighter image. In another example, data acquisition can be set up so that the shorter T₂ times (induced by the inventive MRI assay) appear brighter in the generated image. Alternatively, the T₂ values can be color coded, for example to show up red in the image. As the assay reacts, the shorter T₂ values become more and more red in the generated images over time.

It will be appreciated that a number of different parameters can be manipulated by the MRI operator to build up enough information to construct the images in a number of different ways.

Advantageously, MRI permits the spatially resolved in-situ measurement of protease activity and imaging of cancerous tissue anywhere in the body. The increased in vivo time of the assay also permits detection of much lower protease levels, permitting much earlier detection of cancerous or precancerous cells. In addition, unlike gadolinium contrast agents, a direct contact between the in-vivo water and the nanoplatform MRI contrast agent is not required for observing sufficient MRI-contrasts with the invention, especially in T₂-weighted images.

According to a further embodiment, a method for diagnosing disease progression is provided. In the method, a diagnostic nanoplatform comprising a consensus cleavage sequence for urokinase (SGRSA, SEQ ID NO: 2) is administered, and MRI data is acquired as described above. If urokinase activity is found in the MRI assay, then a diagnostic nanoplatform employing a consensus sequence for matrilysin (MMP-7) is injected intravenously two days later, followed by the acquisition of MRI data. If matrilysin activity is detected, the prognosis is for angiogenesis or metastasis. For confirmation, a nanoplatform comprising a consensus sequence for collagenase (MMP-1) is injected intravenously two days later. If the assay is negative, the prognosis is for angiogenesis. If the assay is positive, the prognosis is for metastasis. If the first urokinase MRI assay was negative, then a collagenase (MMP-1) sensitive MRI imaging drug is given after two days. Advantageously, employing modern MRI instrumentation (B>>2Tesla), a millimeter resolution is achievable when imaging the cancerous tissue that is over-expressing cancer related proteases. This tissue can then either be excised or treated by hyperthermia as sole treatment method or in combination with an anti-cancer drug that is delivered by a thermosensitive nanogel, liposome or micelle. Assay time can also be correlated to prognosis. In general, the more aggressive the cancer, the higher the concentration of a given protease, meaning that observed changes in r₂/r₁ will be faster.

### 2. Light Backscattering

In a further aspect of the invention, the inventive nanoplatforms work on the basis of light backscattering. Light scattering is a physical process where an incoming light wave will be reflected (not absorbed) by a surface. In contrast to fluorescence/phosphorescence detection methods where the absorption and re-emission of light is required, no light absorption occurs during scattering. This also means that the frequency of the scattered electromagnetic wave remains the same. For macroscopic surfaces, the reflection behavior can be described by the law of reflection. For nanoscopic particles however, reflection is a much more complex process as previously discussed. Preferably, the nanoplatform assays can be performed in vitro and in vivo. The light backscattering assay is particularly advantageous for detection and imaging of surface cancers such as melanomas.

a. In Vitro Methods

The nanoplatform assays may be used to detect protease activity in a fluid sample comprising a biological fluid, such as urine or blood samples of a mammal. In one aspect, a urine sample is collected from the mammal and physically mixed with a linked nanoplatform assay. Preferably, the concentration of the nanoplatform in the urine is from about 10 to about 1,000 μg of nanoplatform per ml of urine, and more preferably from about 50 to about 250 μg of nanoplatform per ml of urine. Excitation is preferably performed with an energy source of appropriate wavelength selected from the group consisting of a polychromatic light source, laser, and laser-diode. The wavelength used will depend upon the particles used in the nanoplatform assembly. Preferably, the wavelength ranges between about 200 nm and about 1,000 nm. The backscattered light will have the same frequency than the incoming energy source. The loss of the backscattered signals as the protease in the urine sample cleaves the oligopeptide linkages will be observed as a change in the optical extinction over a time period of from about 30 seconds to about 24 hours, and more preferably from about 2 minutes to about 1 hour. In the presence of the protease, a typical change in the optical extinction of about 0.001 to about 1 will be observed. Thus, in the inventive method, this change in the optical extinction preferably indicates the presence of a cancerous or precancerous cell in the mammal. Blood can be collected from the mammal and analyzed in the same manner as urine discussed above.

These assay results (from the biological fluid) can then be correlated with a prognosis for cancer progression, based upon the specific protease activity detected, as discussed above with regard to the preferred proteases, uPA, MMP-1, MMP-2, and MMP-7, or based upon the speed of the assay, as discussed below.

b. In Vivo Methods

In an alternative embodiment, detection of protease activity using the linked nanoplatforms may be done in vivo in a mammal. The diagnostic nanoplatform assay, or composition comprising the assay, is preferably administered using a pharmaceutically-acceptable carrier (i.e., buffer or liposome). The assay can be administered intravenously by injection into the bloodstream. Alternatively, the assay dissolved in an aqueous buffer (e.g., phosphate buffered saline (PBS)) can be administered by injection to a localized region, such as directly into or near the tumor site. The nanoplatform is preferably utilized at a concentration of from about 100 to about 5,000 μg per ml of PBS, and more preferably from about 200 to about 500 μg per ml of PBS. Liposomal delivery may also be used, including thermolabile liposomes. Cellular delivery can also be used.

Once the linked nanoplatform assay is in the vicinity of the cancerous tissue, excitation will be directed to the region of interest using an energy source selected from the group consisting of a polychromatic light source, laser, and laser diode. As the light- or laser-beam enters the tissue, the backscattered light is preferably recorded via a fiberoptic device. The backscattered light will have the same frequency as the incoming light, and the signal will be much stronger (up to from about 2 to about 100 times stronger) in the presence of the linked nanoplatforms than in their absence. Thus, the signal is preferably stronger in the cancerous tissues where the nanoplatforms aggregate than in the surrounding healthy tissue. The loss of the backscattered signals as the protease in the cancerous tissue cleaves the oligopeptide linkages will be observed as a change in the optical extinction over a time period of from about 30 seconds to about 24 hours, and more preferably from about 2 minutes to about 1 hour. Notably, the signal will still be stronger than in the healthy tissue. In the presence of the protease, a typical change in the optical extinction of about 0.05 to about 1 will be observed. Thus, in the inventive method, this change in the optical extinction preferably indicates the presence of a cancerous or precancerous cell in the mammal. The assay results can then be correlated with a prognosis for cancer progression, based upon the protease activity detected, as discussed in more detail below.

Using either sensor method (in vitro or in vivo), the assay time of the present invention is dependent upon the concentration of protease present in the sample or tissue. The cleavage speeds will increase by 3-5 times per order of magnitude of increase in protease concentration. In the presence of an aggressive tumor, assay time can be as fast as a fraction of a second. In healthy tissue, it can take about 24 hours for activity to be detected. Thus, the faster the assay, the more aggressive the tumor, and the greater the likelihood of metastatic potential of the tumor. The use of protease-specific oligopeptides for the construction of a nanoparticle-based in vivo nanosensors for the determination of the metastatic potential of solid tumors permits the physician and surgeon to target the more advanced tumors first. Preferably, when the assay is directly injected into the tumor region (or suspected tumor region), results can be determined about 30 minutes after injection. When the assay is administered intravenously, the results can be read within about 1 hour after administration of the IV (to permit the assay to reach the target region), and up to 24 hours after administration. In either case, once the assay is in the vicinity of the tumor, protease activity detected within 10 minutes can be correlated with a high probability that the tumor is aggressive. Preferably, if no activity is detected within the first 30 minutes, there is a very low probability that the tumor is aggressive. Likewise, for in vitro testing protease activity detected within 10 minutes can be correlated with a high probability that the tumor is aggressive, whereas no activity within the first 30 minutes after contacting the sample with the assay can be correlated with a very low probability that the tumor is aggressive. This reaction rate provides a distinct advantage over known detection methods which take several hours for assay completion (and results).

### 3. FRET-Based Sensors

The nanoplatforms are also suitable for detection methods based upon surface plasmon resonance and Forster resonance energy transfer (FRET) between non-identical particles (i.e., nanoparticles or a nanoparticle and porphyrin). FRET describes energy transfer between two particles. Surface plasmon resonance is used to excite the particles. A donor particle initially in its excited state, may transfer this energy to an acceptor particle in close proximity through nonradiative dipole-dipole coupling. Briefly, while the particles are bound by the oligopeptide, emission from the acceptor is observed upon excitation of the donor particle. Once the enzyme cleaves the linkage between the particles, FRET change is observed, and the emission spectra changes. Only the donor emission is observed. In more detail, if both particles are within the so-called Forster-distance, energy transfer occurs between the two particles and a red-shift in absorbance and emission is observed. During this ultrafast process, the energy of the electronically excited state or surface plasmon of the first particle is at least partially transferred to the second particle. Under these conditions, light is emitted from the second particle. However, once the bond between the two particles is cleaved by the enzyme, light is emitted only from the first particle and a distinct blue-shift in absorption and emission is observed. This is because the distance between both particles greatly increases.

a. In Vitro Methods

The nanoplatforms may be used to detect protease activity in a fluid sample comprising a biological fluid, such as urine or blood samples of a mammal. In one aspect, a urine sample is collected from the mammal and physically mixed with the nanoplatform assay. Preferably, the concentration of the luminophore in the urine is from about 1×10⁻⁴M to about 1×10⁻¹⁰ M, and more preferably from about 1×10⁻⁵M to about 1×10⁻⁸M. Excitation is preferably performed with an energy source of appropriate wavelength selected from the group consisting of a tungsten lamp, laser diode, and laser. The wavelength used will depend upon the particles used in the nanoplatform assembly. Preferably, the wavelength ranges between about 400 nm and about 1,000 nm, and more preferably between about 500 nm and 800 nm. The changes in absorption and emission of the particles as the protease in the urine sample cleaves the oligopeptide linkers will be observed over a time period of from about 1 second to about 30 minutes, and preferably from about 30 seconds to about 10 minutes, when in the presence of an aggressive tumor. In the presence of the protease, a typical absorption and emission blue-shift of between about 5 and about 200 nm will be observed. Thus, in the inventive method, a blue-shift in absorption or emission spectrum maximum between 5 and 200 nm preferably indicates the presence of a cancerous or precancerous cell in the mammal.

Blood can be collected from the mammal and analyzed like urine discussed above. Preferably, the concentration of the assay in the blood sample is from about 1×10⁻⁴ M to about 1×10¹⁰ M, and more preferably from about 1×10⁻⁵ M to about 1×10⁻⁸ M. The wavelength used will depend upon the particles used in the nanoplatform assembly. Preferably, the wavelength ranges between about 500 nm and about 1,000 nm, and more preferably between about 600 nm and 800 nm. More preferably, excitation is performed using multi-photon excitation at a wavelength of about 800 nm with a Ti-sapphire-laser because of the strong self-absorption of blood. Changes in emission will be observed over a time period of from about 1 second to about 30 minutes, and preferably from about 30 seconds to about 10 minutes, when in the presence of an aggressive tumor. As with urine, in the presence of the protease in the blood, a typical emission blue-shift of between about 5 and about 200 nm will be observed. This preferably indicates the presence of a cancerous or precancerous cell in the mammal.

These assay results (from urine or blood) can then be correlated with a prognosis for cancer progression, based upon the specific protease activity detected or the speed of the assay, as discussed above.

The assay can also be used to monitor progress of cancer treatment in a patient over time by determining the presence and level of various proteases in the blood or urine of a patient during or between treatments. Assays can be run on a daily basis while the patient is undergoing treatment and the protease activity levels compared between the initial and subsequent levels. Likewise, assays may be performed periodically (i.e., on a monthly basis) after a patient has gone into remission to facilitate early detection of cancer reoccurrence. Thus, assay can help determine whether the cancer is diminishing or increasing in severity based upon the assay results,

b. In Vivo Methods

The nanoplatform assay can be administered as described above for the light backscattering detection methods. Once the assay is in the vicinity of the cancerous cells, one or two intersecting Ti:sapphire lasers are preferably used to excite the assay. Other suitable excitation sources include Nd:YAG-lasers (first harmonic at 1,064 nm), and any kind of dye-laser, powered by the second harmonic of the Nd:YAG-laser at 532 nm. The light emission from the assay will then be analyzed using a camera, microscope, or confocal microscope. The light emitted from the cancerous regions has a different color than the light emitted from the healthy tissue regions due to the higher activity of the target proteases in the cancerous regions. Advantageously, the cancerous tissue is then visibly discernible to an oncologist or surgeon. For example, the nanoplatforms can be used to identify the boundary of the cancerous tissue to facilitate removal of cancerous tissue and tumors while preserving as much healthy tissue as possible. Preferably, the Ti:sapphire laser is tuned to a wavelength of about 830 nm for the multi-photon excitation so that only the light emission, but not the excitation can be observed. The assay results can then be correlated with a prognosis for cancer progression, based upon the protease activity detected.

4. Light-Switch-Based Sensors

In another aspect, the assays utilize a nanoplatform comprise a nanoparticle having one or more protective layer bound via an oligopeptide linkage to a porphyrin or other organic or inorganic luminophore. In this method, the surface plasmon of the core/shell nanoparticle is able to quench the excited state emission spectra from the linked porphyrin. Once the protease cleaves the consensus sequence, the porphyrin is released and lights up, referred to herein as an “enzyme-triggered light switch.” Advantageously, the appearance of a new luminescence/fluorescence band allows for much more sensitive detection. Preferably, excitation is performed at a wavelength of from about 400 nm to about 500 nm (monophotonic) or from about 800 nm to about 900 nm (multi-photonic). Excitation of porphyrins is preferably performed using tri-photonic excitation with Ti:sapphire laser at 870 nm. The emission from the assay will then be analyzed using a camera, microscope, or confocal microscope. The light-switch-based sensors can be utilized in the exact same procedure (in vitro or in vivo) as the discussed above with regard to the FRET-based sensors. Using either sensor method (in vitro or in vivo), the assay time of the present invention is dependent upon the concentration of protease present in the sample or tissue, and can be directly correlated to the severity of the cancer as discussed for the light backscattering methods.

This method is particularly suited for monitoring cancer progression and treatment progress. In one aspect, a first sample (such as urine) is collected from a mammal diagnosed with cancer and mixed with the nanoplatform assay. The assay is then excited using a suitable excitation source and the emission (or absorption) spectrum is analyzed. The rate of enzyme hydrolysis can then be correlated with the severity of the cancer, as described herein. Samples can also be collected from the patient over time and compared to determine whether the cancer is increasing or decreasing in severity. For example, a first sample can be collected from a patient upon the initial diagnosis of cancer and subjected to a first assay. After undergoing a first course of treatment, a second sample can be collected from the patient and subjected to a second assay. The results can then be compared to the results from the first assay to determine if enzyme activity levels have increased or decreased. If the levels have decreased, the prognosis is that the treatment is working and the course of treatment should be maintained (or perhaps decreased). If the levels have increased, the prognosis is that the treatment needs to be increased or altered. If levels decrease dramatically, the prognosis might be for remission and treatment can be stopped. The assay can then be performed periodically to detect for the reoccurrence of the cancer. The assay results can therefore determine whether a particular course of treatment is effective for treating the cancer.

The light switch method is also suitable for identifying the boundary of cancerous tissue and tumors during surgery to enable more precise tissue excision, as described above with respect to FRET-based sensors.

### Therapeutic Treatment

Hyperthermia (heating cells to a few degrees above their growth temperature) can lead to cell death (reproductive capacity), and can also enhance the sensitivity of cells for radiation and chemotherapeutics. Although many cancer cells are slightly more susceptible to hyperthermia than healthy cells, the latter often share the same fate when an entire portion of the body is indiscriminately heated. Therefore, the development of methods to selectively target hyperthermia treatment in cancer cells remains one of the challenges in this field. This is equally important when attempting to treat solid tumors within the human body, as well as for the treatment of metastatic cancers.

In the inventive method, the therapeutic (unlinked) nanoplatform or composition comprising the nanoplatform is administered to a mammal, preferably using a pharmaceutically-acceptable carrier. The nanoplatform can be administered by injection to a localized region, such as directly into or near the tumor site. The nanoplatform can be administered intravenously by injection into the bloodstream. The amount of nanoplatform in each dose is preferably from about 0.001 to about 0.10 g per kg of the patient's weight, and more preferably from about 0.010 to about 0.025 g per kg of the patient's weight. Liposomal delivery of the nanoplatform to the cancerous tissue may also be used, including thermolabile liposomes. However, cellular delivery of the nanoplatforms to the cancerous tissue is particularly preferred for hyperthermia treatment. When heated, the delivery cells perish and release their cargo directly to the cancerous tissue.

Once the nanoplatform has been taken up by the cancer cells and located in the cancer tissue, the target region of interest is heated using magnetic A/C-excitation. Excitation is preferably performed at frequencies ranging from about 50 to about 500 kHz, and preferably from about 100 to about 300 kHz. Preferably, A/C magnetic heating begins from about 12 hours to about three days after nanoplatform delivery to the cancerous tissue. Magnetic A/C-excitation raises the temperature of the nanoplatform, this heat is then dissipated into and raises the temperature of the cancerous tissue, resulting in growth inhibition, and cell death. Because the nanoplatforms are selectively taken up by the target cancerous tissue, the heat remains relatively confined to the target tissue minimizing damage to surrounding healthy tissue. Preferably, the target tissue is heated to a temperature of at least about 40° C., more preferably from about 42° C. to about 60° C., and even more preferably from about 45° C. to about 50° C. The duration of the treatment preferably lasts from about 10 minutes to about 2 hours, and more preferably from about 10 minutes to about 1 hour. The temperature and duration of heating can be modified depending upon the treatment goal.

At high temperatures (>60° C.) resulting from plasmonic and intense A/C-magnetic hyperthermia, partial carbonization, massive protein denaturation and a partial dissolution of cell and mitochondrial membranes in the surrounding buffer solution are observed. These processes result in necrosis (uncontrolled, premature cell death), which is characterized by cell swelling, chromatin digestion, and disruption of the plasma membrane and organelle membranes, followed by extensive DNA hydrolysis, vacuolation of the endoplasmic reticulum, organelle breakdown (especially mitochondria and lysosomes) and, eventually, cell lysis. Damage to the lysosomes usually triggers the release of lysosomal cysteine proteinases (caspases and other proteases), which first lyse many vital cell structures and then are released from the dead cell. They can trigger a chain reaction of further cell deaths of neighboring cells.

When heated to medium temperatures of from about 43° C. to about 45° C., vital proteins of the cancer cell become damaged (e.g. misfolded) and/or the cell membrane partially dissolves in the surrounding aqueous medium. The influx of calcium from the interstitium and endoplasmatic reticulum synchronizes the mass exodus of cytochrome c from the mitochondria. These deviations from the “normal” metabolism of a cancer cell can eventually lead to apoptosis (programmed cell death). After hyperthermia, significant increases in TRAIL ((tumor necrosis factor (TNF)-related apoptosis-inducing ligand) is observed. In short, hyperthermia induces apoptosis in cells that is mediated by caspase-3 and other caspases as a result of activation of cell-death membrane receptors of the tumor-necrosis-factor family. For hyperthermia treatment of cancerous tissue, apoptosis is preferred to necrosis because it is less damaging to surrounding healthy tissue.

It has been found that if temperatures of between about 43° C. and about 45° C. are retained for an extended period of time (greater than about 1 hour, and preferably between about 1 hour and about 2 hours), the anti-tumor immune response can be markedly enhanced. In addition, the heat shock proteins (hsp) which are produced in abundant quantities in cells exposed to heat, are potent immune modulators and can lead to stimulation of both the innate and adaptive immune responses to tumors. Immunostimulation by hyperthermia involves both direct effects of heat on the behavior of immune cells as well as indirect effects mediated through hsp release.

For optimal heating, the nanoparticles utilized in the nanoplatforms, preferably have a very narrow size/mass distribution as previously described. In addition, the nanoparticles preferably feature a strongly paramagnetic iron-core. Compared to existing superparamagnetic iron oxides for hyperthermia applications, superparamagnetic iron possesses a higher magnetic moment and a higher saturation magnetization. This permits both lower concentrations of the nanoplatforms in the tissue than existing treatments and shorter A/C-magnetic heating times during the treatment of patients. Even more preferably, the nanoparticles also feature a Fe₃O₄ shell around the iron core. Particularly preferred therapeutic nanoplatforms comprise a Fe/Fe₃O₄ core/shell nanoparticle surrounded by a siloxane protecting layer and ligand monolayer. An important factor for A/C magnetic hyperthermia is the specific absorption rate or SAR of the nanoparticle, which is determined by SAR=C*ΔT/Δt, where C is the specific heat capacity of the sample and T and t are the temperature and time, respectively. Thus, the therapeutic nanoplatforms will preferably have a specific absorption rate (SAR) of at least about 50 W/g, preferably from about 100 to about 5,000 W/g, and more preferably from about 1,500 to about 2,000 Wig.

SAR is very sensitive to the material properties. While in multi-domain particles the dominant heating is hysteresis loss due to the movement of domain walls, it is not so in case of small particles. The two main contributing mechanisms of SAR in single domain magnetic nanoparticles are the Brownian (rotation of the entire nanoparticle) and Neel (random flipping of the spin without rotation of the particle) relaxations. The transition between the two mechanisms occurs between 5-12 nm for various materials, but it also varies with frequency. The preferred nanoparticles will be dominated by Néel relaxation due to the superparamagnetic nature of the iron(0)-core.

The human body tolerates Fe²⁺ and Fe³⁺ much better than many other metals (e.g. Cd²⁺). The tolerable daily upper intake level (UL) for iron is 45 mg per day for adults. If an imaging or treatment procedure requires the intake of more iron, chelation treatment is feasible. The most widely used iron chelator, desferrioxamine, removes up to 70 mg of iron per day from the bloodstream of an adult. Assuming that the complete biocorrosion of the theranostic nanoparticles is 5 days, 575 mg of iron can be given at once for imaging or treatment. If the additional siloxane-protection layer is present, the lifetime of the Fe/Fe₃O₄/ASOX/stealth nanoparticles is increased, and the dosage of iron in the nanoplatforms can be increased up to about 2.3 g for a single dose. In addition, an overdose of Fe³⁺ can greatly increase the amount of reactive oxygen species (ROS) in the body further enhancing the tumor inhibition.

Advantageously, the hyperthermia treatment could directly follow the imaging and detection methods described above. That is, the same nanoplatforms or assays utilized for imaging and detection in a patient can then be used to immediately treat the detected cancerous tissue without the administration of any additional nanoplatforms or other agents.

### EXAMPLES

The following examples set forth preferred methods in accordance with the invention. It is to be understood, however, that these examples are provided by way of illustration and nothing therein should be taken as a limitation upon the overall scope of the invention.

### Example 1

### Synthesis of Organic Stealth Ligands

In this Example, three different ligands for the stealth coating of the nanoparticles are synthesized. Analysis of each reaction product was done by proton NMR (¹H NMR) and/or carbon-13 NMR (¹³C NMR), employing a 400 MHz NMR spectrometer (Varian; Kans. State University), and by Electrospray Ionization Mass Spectrometry (MS-ESI), employing a hybrid triple quadrupole/linear ion trap mass spectrometer (4000 Q-TRAP®, Applied Biosystems; Foster City, Calif.) with an electrospray source.

### A. Ligand A Synthesis

1. Boc-Protection of Dopamine

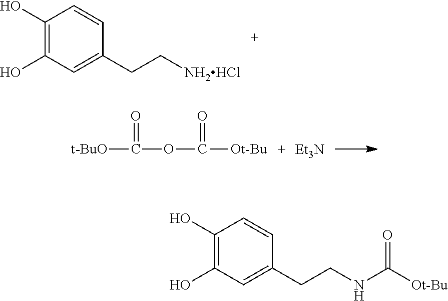

A solution of dopamine (310 mg, 1.63 mmol) in methanol (8 ml) was prepared and stirred under N₂ for 5 minutes. 1.8 mmol triethylamine (TEA) was added to the solution followed by Boc-anhydride (393 mg, 1.8 mmol) The mixture was stirred under N₂ for 12 hours. The solvent was then removed under reduced pressure. The remaining residue was dissolved in 40 ml of CH₂Cl₂ and washed three times with 5 ml of each of 1.0 N HCl and brine. The organic layer was then dried over anhydrous Na₂SO₄. After filtration, the organic phase was kept at −5° C. for 3 hours. A white precipitate came out and was collected by filtration. Total Yield 85%.

¹H NMR spectrum (400 MHz, DMSO-d6) δ: 1.73 (s, 9H); 2.48 (t, 2H); 3.02 (q, 2H); 6.40 (d, 1H); 6.54 (s, 1H); 6.61 (d, 1H); 6.83 (t, 1H); 6.85 (s, 1H); 6.76 (s, 1H).

2. Benzyl-Protection of Boc-Dopamine

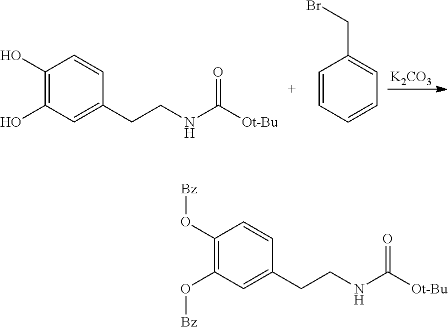

3.47 grams of Boc-protected dopamine were dissolved in 100 ml of dimethylformamide (DMF). 12.6 grams of K₂CO₃ were then added, and the system was protected under N₂. Next, 4.69 grams of (2 eq.) benzyl bromide were added dropwise to the solution. The mixture was stirred at room temperature for 24 hours without light. The resulting solid was then removed by filtering through a short pad of celite, and the filter-cake was washed three times with 100 ml of ether. The combined filtrate and washing solution were washed three times with ice-water (50 ml) and brine (15 ml). The organic layer was dried over anhydrous Na₂SO₄ and concentrated to 150 ml. After setting at −5° C. for 5 hours, a white precipitate came out and was collected by vacuum filtration. Total Yield 90%.

¹H NMR (400 MHz, CDCl₃) δ: 1.45 (s, 9H); 2.70 (t, 2H); 3.31 (q, 2H); 4.49 (s, 1H); 5.15 (d, 4H); 6.71 (d, 1H); 6.80 (s, 1H); 6.88 (d, 1H); 7.32 (t, 2H); 7.37 (t, 4H); 7.45 (d, 4H).

3. Deprotection of Boc-Group

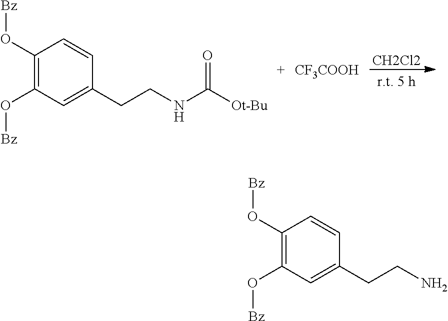

4.3 grams of benzyl-protected Boc-dopamine were dissolved in 150 ml of 5% trifluoroacetic acid (TFA) CH₂Cl₂ solution and stirred at room temperature for 5 hours. The solvent was removed under vacuum and clear oil was obtained. Total Yield 100% yield.

¹H NMR (400 MHz, CDCl₃) δ: 2.79 (t, 2H); 3.08 (m, 2H); 5.11 (s, 4H); 6.68 (d, 1H); 6.75 (s, 1H); 6.90 (d, 1H); 7.32 (t, 2H); 7.35 (t, 4H); 7.42 (d, 4H). ¹³C NMR (400 MHz, CDCl₃) δ: 32.90; 41.85; 71.50; 72.00; 115.60; 116.25; 122.30; 127.60; 127.85; 128.35; 128.45; 128.63; 128.85; 136.70; 136.85; 148.45; 149.00; 160.88; 161.20; 161.58; 161.90.

4. Amid Formation

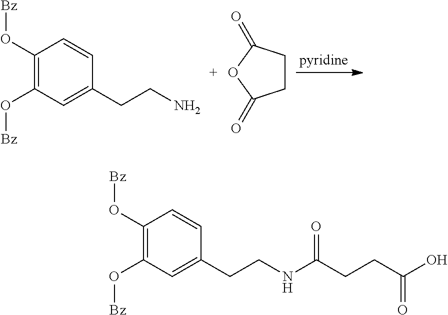

1.43 grams of benzyl-protected dopamine and 0.43 grams of succinic anhydride (1:1 molar ratio) were dissolved in 6 ml of pyridine. The solution was stirred at room temperature for 5 hours. The solvent was removed by co-evaporation with toluene (5×5 ml). A white solid was obtained and washed three times with CH₂Cl₂. After drying under vacuum, 1.4 grams of product were obtained. Total Yield 75%.

¹H NMR (400 MHz, DMSO-d6) δ: 2.29 (t, 2H); 2.42 (t, 2H); 2.60 (t, 2H); 3.21 (q, 2H); 5.09 (d, 4H); 6.71 (d, 1H); 6.94 (s, 1H); 6.96 (d, 1H); 7.32 (t, 2H); 7.38 (d, 4H); 7.45 (t, 4H); 7.90 (t, 1H); 12.08 (s, 1H). MS-ESI⁺: m/z 434.2. Molecular weight: 433.5.

5. Ester Formation

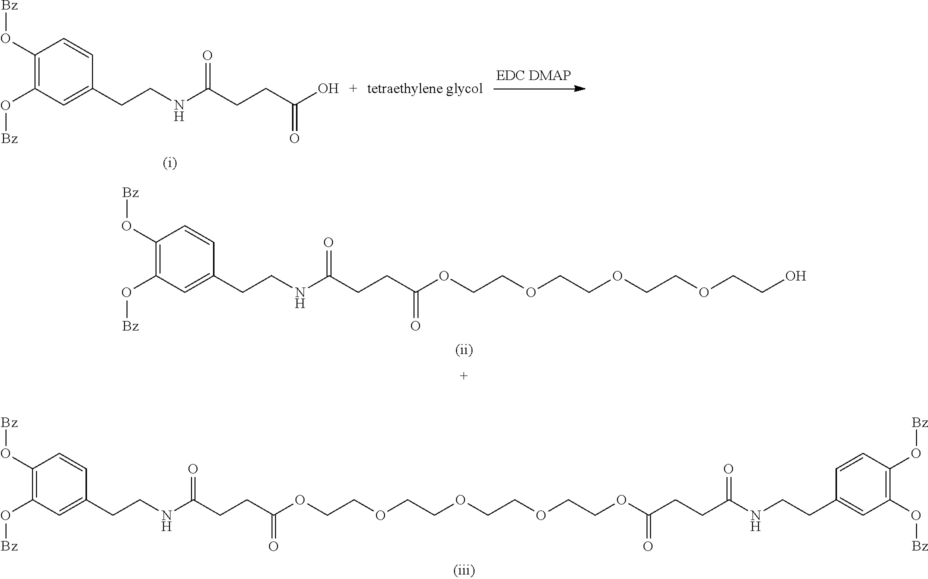

0.964 grams of the reaction product from step 4 above and 0.426 grams of 1-ethyl-3-(3-dimethylaminopropyl) carbodiimide (EDC) (1:1 molar ratio) were dissolved in 100 ml of CH₂Cl₂ and stirred at room temperature for 10 minutes. Next, 0.433 grams of tetraethylene glycol were added to the solution followed by 5 mg of dimethylaminopyridine (DMAP). After stirring for 12 hours at room temperature, the organic phase was washed three times with 10% H₃PO₄ solution (10 ml), water (10 ml), and brine (10 ml). The organic phase was then dried over anhydrous Mg₂SO₄. After removing the solvent under vacuum, the residue was loaded on column and eluted with 1:1 acetone/methylene chloride. 0.42 grams of product ii (benzyl-protected dopamine-based tetraethylene glycol) were obtained. Total Yield 40%. 0.4 grams of side product iii was also isolated.

¹H NMR for product ii (400 MHz, CDCl₃) δ: 2.39 (t, 2H); 2.57 (t, 1H); 2.70 (q, 4H); 3.44 (q, 2H); 3.60 (t, 2H); 3.65 (broad 12H); 4.24 (t, 2H); 5.15 (d, 4H); 5.74 (t, 1H); 6.71 (d, 1H); 6.81 (s, 1H); 6.89 (d, 1H); 7.31 (t, 2H); 7.37 (t, 4H); 7.46 (d, 4H). MS-ESL: m/z 610.4. Molecular weight 609.3.

6. De-Benzylation to Produce Ligand A

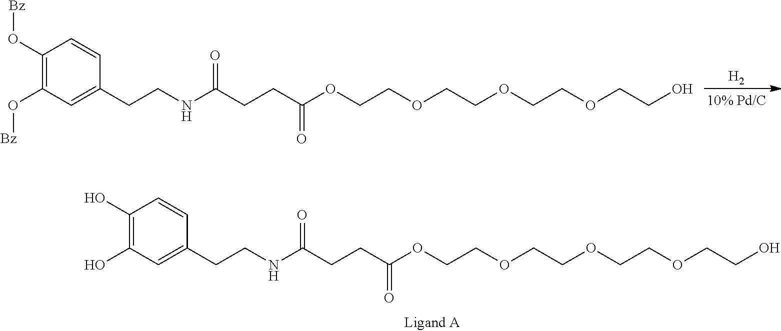

0.34 grams of benzyl-protected dopamine-based tetraethylene glycol (ii) were dissolved in 50 ml of methanol. Next, 77 mg of palladium on carbon (Pd/C) were added under N₂. After evacuating three times, 1 atm. H₂ was applied and the mixture was stirred for 24 hours at room temperature. The catalyst was removed by filtering through a short pad of celite. The solvent was then removed under vacuum, resulting in 0.23 grams of product (Ligand A). Total Yield 100%.

¹H NMR (400 MHz, DMSO-d6) δ: 2.33 (t, 2H); 2.48 (q, 2H); 3.15 (broad multiplet, 4H); 3.41 (t, 2H); 3.49 (t, 2H); 3.51 (broad multiplet, 8H); 3.59 (t, 2H); 4.11 (t, 2H); 6.41 (d, 1H); 6.55 (s, 1H); 6.61 (d, 1H).

### B. Ligand B Synthesis

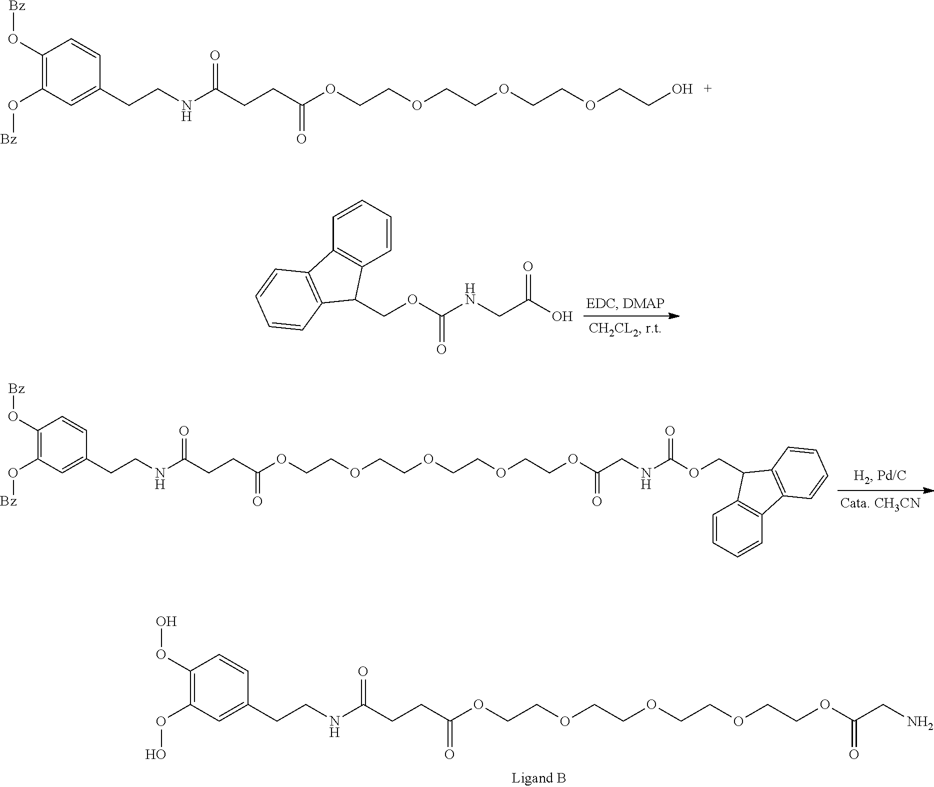

1.0 gram of benzyl-protected dopamine-based tetraethylene glycol (product ii from A.5. above) was treated with 1 equiv. of Fmoc-Glycine and 1.2 equiv. of EDC in the presence of 0.020 grams of DMAP to give over 95% coupled product. The benzyl and Fmoc groups were deprotected at the same time with hydrogen/palladium on carbon (H₂/Pd(C)) in the presence of 10 ml of CH₃CN. The catalyst was removed by filtering through a short pad of celite. The solvent was then removed under vacuum, resulting in Ligand B. Total Yield 35%.

¹H NMR (400 MHz, DMSO-d6) d: 2.33 (t, 2H); 2.46 (q, 2H); 3.14 (q, 2H); 3.41 (t, 2H); 3.49 (t, 4H); 3.51 (broad multiplet, 8H); 3.59 (t, 2H); 4.10 (t, 2H); 4.57 (t, 2H); 6.43 (d, 1H); 6.55 (s, 1H); 6.61 (d, 1H); 7.90 (t, 1H); 8.62 (s, 1H); 8.73 (s, 1H). ¹³C NMR (400 MHz, DMSO-d6) δ: 28.98; 29.85; 34.73; 60.25; 63.33; 68.30; 72.38; 115.49; 115.96; 119.22; 130.25; 143.54; 145.07; 170.48; 172.48.

### C. Ligand C Synthesis

1. Urethane Formation

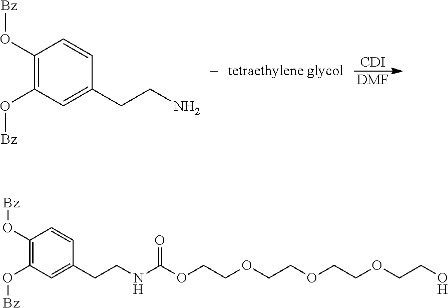

1.43 grams of benzyl-protected dopamine (from A.3. above) were dissolved in 5 ml of ahydrous DMF, along with 0.83 grams of tetraethylene glycol (1:1 ratio) and 0.50 grams of carbonyl-bis-imidazole (CDI). The solution was stirred at room temperature for 1 hour and then at 60° C. for 4 hours. The solvent was then removed by co-evaporation with toluene (5×5 ml). A white solid was obtained and washed with CH₂Cl₂ 3 times. After drying in a vacuum, 1.66 grams of product were obtained. Total Yield: 70%.

¹H NMR (400 MHz, CDCl₃ δ: 2.40 (s, 1H); 2.88 (m, 4H); 3.26 (q, 2H); 3.68 (t, 2H); 3.66 (broad 12H); 4.25 (t, 2H); 5.18 (d, 4H); 5.74 (t, 1H); 6.71 (d, 1H); 6.81 (s, 1H); 6.89 (d, 1H); 7.31 (t, 2H); 7.37 (t, 4H); 7.46 (d, 4H), 8.24 (s, 1H). MS-ESI⁺: m/z 553.2.

2. Deprotection to Produce Ligand C

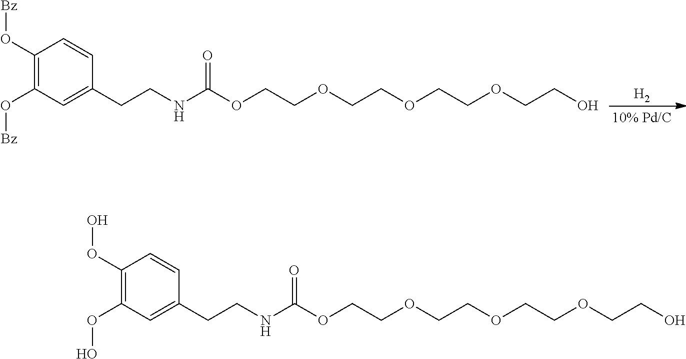

0.35 grams of benzyl-protected dopamine-based tetraethylene glycol ligand were dissolved in 50 ml methanol. 77 mg Pd/C was added under N₂. After evacuating three times, 1 atm. H₂ was applied and the mixture was stirred for 24 hours at room temperature. The catalyst was removed by filtering through a short pad of celite. After removing the solvent under vacuum, 0.235 grams of product (Ligand C) were obtained. Total Yield: 98%.

¹H NMR (400 MHz, DMSO-d6) δ: 2.43 (t, 2H); 3.45 (t, 2H); 3.49 (t, 2H); 3.54 (broad multiplet, 10H); 3.60 (t, 2H); 4.11 (t, 2H); 6.41 (d, 1H); 6.55 (s, 1H); 6.61 (d, 1H).

### Example 2

### Synthesis of Non-Metalated Porphyrin

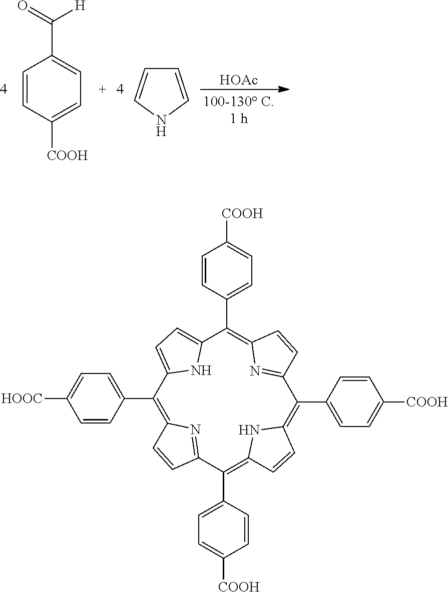

In this Example, a non-metalated tetracarboxyphenyl porphyrin (TCPP) was synthesized. First, 1.50 grams of 4-carboxybenzaldehyde were dissolved in 80 ml of acetic acid. The solution was warmed to 100° C., followed by the dropwise addition of a solution of 0.67 grams of pyrrole in 10 ml of acetic acid over a period of 20 minutes. Upon completion of the addition, the resulting solution was warmed up to 130° C. slowly and kept at 130′C for 1 hour. The mixture was then cooled to 80° C. Next, 100 ml of 95% ethanol were added and the temperature was lowered to room temperature while stirring for 3 hours. The mixture was then stored at −15° C. for 24 hours. A purple solid was collected by vacuum filtration. The filter cake was then washed three times with 5 ml of cold 50/50 ethanol/acetic acid, and dried under high vacuum (oil pump) overnight. 0.51 grams of pure product were obtained. Total Yield 25.5%.

¹H NMR (400 MHz, DMSO-d6) δ: −2.94 (s, 2H); 8.35 (d, 8H); 8.39 (d, 8H); 8.86 (s, 8H); 13.31 (s, 4H). ¹³C NMR (400 MHz, DMSO-d6) δ: 119.31; 127.90; 130.51; 134.44; 145.42; 167.46. MS-ESI⁺: m/z 791.2. Molecular weight 790.2.

### Example 3

### Alternative Synthesis Method for Ligand A

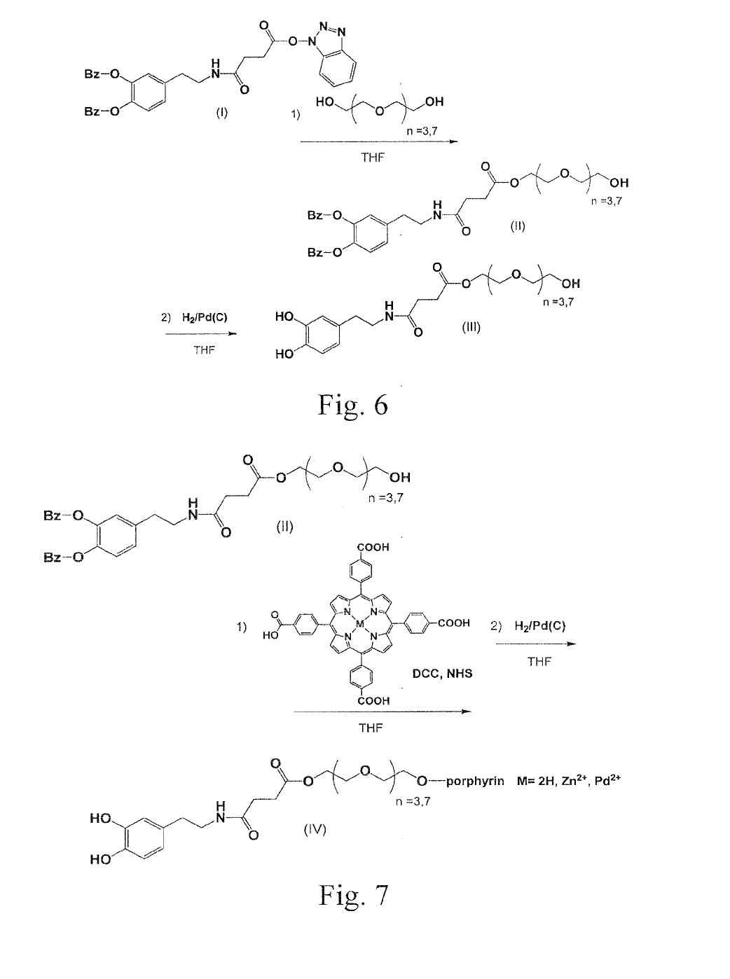

The synthesis starts with the benzyl-protected dopamine, which reacts first with succinic anhydride and then with dicyclohexyl-carbodiimide (DCC) and N-hydroxy-benzotriazole (HOBT) to selectively form a HOBT-active ester (I). This active ester reacts with commercially available tetraethylene glycol or octaethylene glycol to compound (II), which is then deprotected with H₂/Pd(C) in tetrahydrofuran (THF), resulting in compound (III). This reaction scheme is shown in FIG. 6.

Purification of all stages can be achieved by descending column chromatography using neutral silica as stationary phase and n-hexane/ethyl acetate as eluent. According to molecular modeling the octaethylene glycol ligand has a length of 3.7 nm, whereas the tetraethylene glycol ligand is 2.5 nm in length.

The porphyrin can be attached to the ligand prior to stabilization of the nanoparticle. In this embodiment, compound II can be reacted with metalated (M=Zn²⁺ or Pd²⁺ or non-metalated (M=2H) tetracarboxyphenyl porphyrin (TCPP) using DCC and N-hydroxy-succinimide (NHS) as coupling agents in THF, followed by deprotection with H₂/Pd(C) in THF, as shown in FIG. 7. The resulting compound (IV) can be purified by descending column chromatography or reverse phase HPLC(C18) using H₂O/acetonitrile gradients as mobile phase.

### Example 4

### Stabilization of Fe/Fe₃O₄ Nanoparticles with Dopamine-Based Ligands

In this Example, Fe/Fe₃O₄ core/shell nanoparticles were stabilized using Ligands A and B synthesized in Example 1 above, followed by attachment of the porphyrin synthesized in Example 2. The nanoparticles were obtained from NanoScale Corporation (Manhattan, Kans.). The Fe(0)-core had a diameter of about 5.4 nm. The thickness of the Fe₃O₄ shell was about 1.5 nm.

First, 26 mg of dopamine-based Ligand A and 5 mg of dopamine-based Ligand B were dissolved in 5 ml THF. Next, 10 mg of the Fe/Fe₃O₄ nanoparticles were added, followed by sonicating for 60 minutes. The stabilized nanoparticles were then collected using a magnet. The resulting solid was then washed three times with 1 ml THF, and re-dissolved (dispersed) in 5 ml of THF. The attachment of each ligand is depicted below, where n=3.

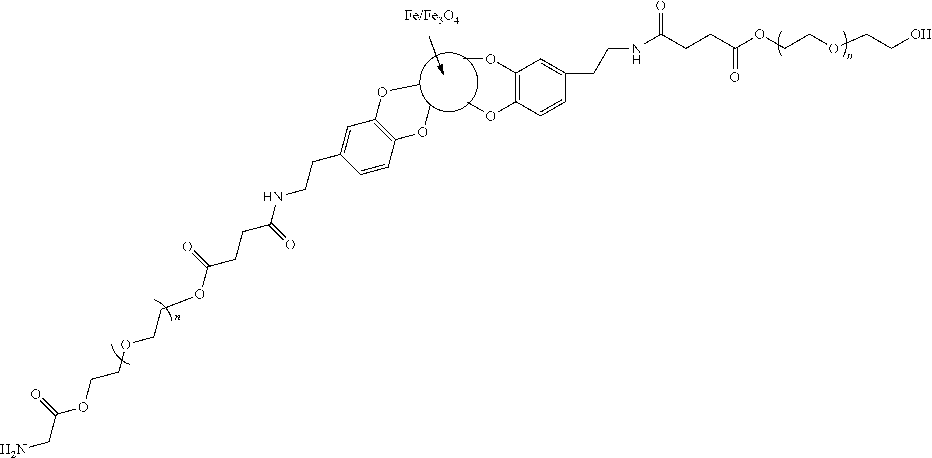

Next, 17 mg of the tetracarboxyphenyl porphyrin (TCPP), synthesized in Example 2 was added to the suspension, along with 2 mg of DMAP and 4 mg of EDC, followed by sonicating for 60 minutes. The solid was collected by magnet and washed with 3 ml of THF until the washing was colorless (about 8 times). The solid was then dried under vacuum. 8.9 mg of solid (stabilized nanoparticles) were obtained. Total Yield 20%. The porphyrin attachment is depicted below.

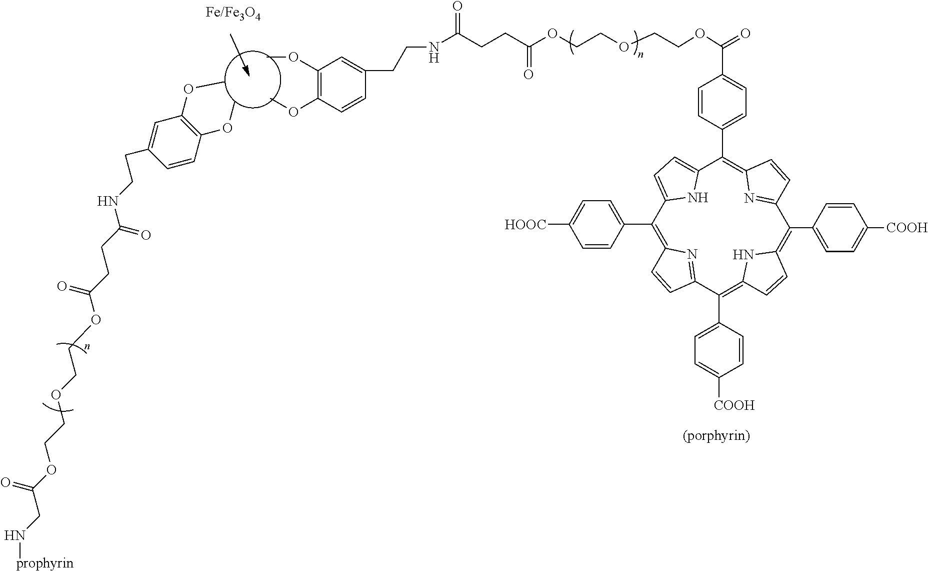

### Example 5

### Modification of Fe/Fe₃O₄ Nanoparticles with Biotin-Labeled Dopamine Based Ligands

In this Example, Fe/Fe₃O₄ core/shell nanoparticles were stabilized using Ligand C synthesized in Example 1 above, followed by attachment of a biotin label. The nanoparticles were obtained from NanoScale Corporation (Manhattan, Kans.). The Fe(0)-core had a diameter of about 5.4 nm. The thickness of the Fe₃O₄ shell was about 1.1 nm.

First, 30 mg of ligand C were dissolved in 5 ml of THF. Next, 10 mg of the Fe/Fe₃O₄ nanoparticles were added, followed by sonicating for 60 minutes. The stabilized nanoparticles were then collected using a 0.5 T iron magnet (Varian). The resulting solid was then washed three times with 1 ml THF, and re-dissolved (dispersed) in 5 ml of THF.

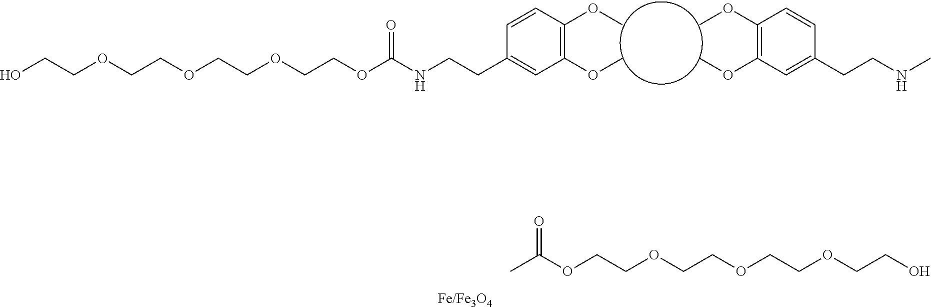

Next, 20 mg of biotin, 2 mg of DMAP, and 4 mg of EDC were added to the suspension and sonicated for 60 minutes. The solid was collected using a magnet and washed with THE (=8 times with 3 ml), until the supernatant was colorless. The solid was dried under vacuum, and 8.7 mg of brown solid were obtained.

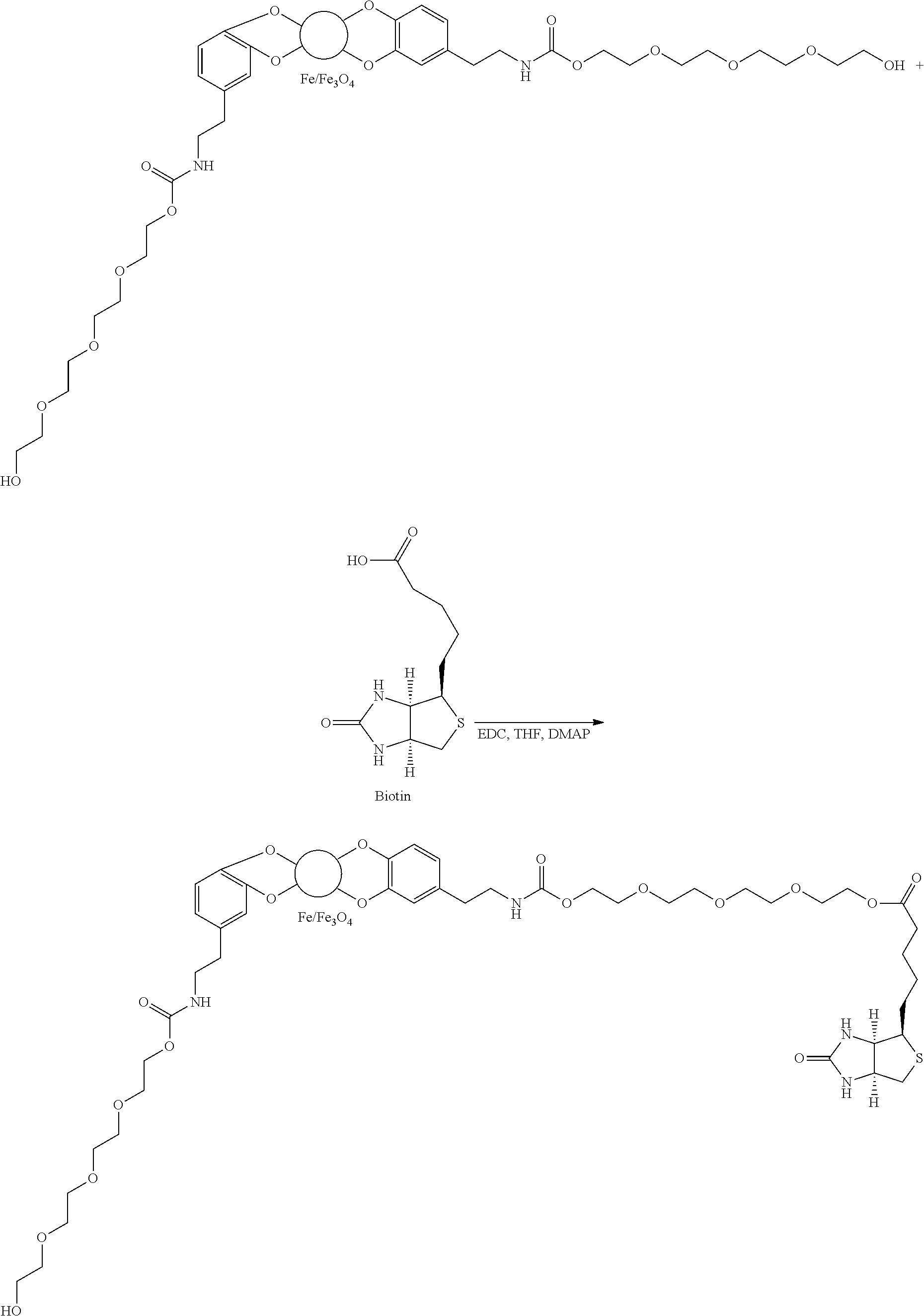

The solubility of the biotin-labeled nanoparticles was then measured. Phosphate buffer (0.1M. pH=6.8) was added dropwise to 0.25 mg of the nanoparticles in a glass cuvette. The suspension was continuously stirred with a micromagnetical stirrer (Fisher). The light scattering of the suspension was recorded at 700 nm. Once the particles have dissolved, the extinction (i.e., light absorption and scattering) at 700 nm decreased to less than E=0.01. The solubility was found to be 105 mg/ml.

### Example 6

### Synthesis of Siloxane-Covered Fe/Fe₃O₄ Nanoparticles

In this Example, Fe/Fe₃O₄ core/shell nanoparticles were coated with an aminosiloxane (ASOX) protection layer. The nanoparticles were obtained from NanoScale Corporation (Manhattan, Kans.). The Fe(0)-core had a diameter of about 5.4 nm. The thickness of the Fe₃O₄ shell was about 1.5 nm.

First, 20 mg of Fe/Fe₃O₄ nanoparticles were suspended in 10 ml THF, followed by sonicating for 30 minutes. The undissolved solid was separated by precipitation through low-speed centrifugation at 1500 rpm. The clear solution was transferred to another test tube and 0.3 ml of 3-aminopropyltriethoxysilane were added to the solution. After sonicating for 10 hours, the nanoparticles were collected using a strong magnet and the solution was carefully removed. After washing with THF (3×5 ml) and drying under vacuum, 7.5 mg of ASOX-protected nanoparticles were collected.

### Example 7

### Linking of Dopamine-Based Ligands to ASOX-Protected Fe/Fe₃O₄ Nanoparticles

In this Example, the Fe/Fe₃O₄-ASOX nanoparticles from Example 5 were coated with the dopamine-based ligands A-C synthesized in Example 1, followed by attachment of porhryins and biotin labels, respectively.

### A. Porphyrin Attachment

First, 26 mg of Ligand A and 5 mg of Ligand B were dissolved in 5 ml THF. Next, 10 mg Fe/Fe₃O₄-ASOX nanoparticles and 3.0 mg of CDI were added, followed by sonicating for 60 minutes. The nanoparticles were collected using a magnet, and the solid was washed with THF (3×1 ml) and re-dissolved (dispersed) in 5 ml THF. Next, 17 mg TCPP porphyrin, 2 mg DMAP, and 4 mg EDC were added to the suspension and sonicated for 60 minutes. The solid was collected using a 0.5 T iron magnet (Varian), and washed with THF (8×3 ml) until the washing was colorless. The solid was dried under vacuum, and 9.0 mg solid was obtained. Solubility in water: 52 mg/ml.

### B. Biotin Labeling

First, 30 mg of Ligand C were dissolved in 5 ml THF. Next, 10 mg Fe/Fe₃O₄-ASOX nanoparticles and 3.0 mg of CDI were added, followed by sonicating for 60 minutes. The nanoparticles were collected using a 0.5 T iron magnet (Varian). The solid was washed with THF (3×1 ml) and re-dissolved (dispersed) in 5 ml THF. Then, 20 mg biotin, 2 mg DMAP, and 4 mg EDC were added to the suspension and sonicated for 60 minutes. The solid was magnetically collected and washed with THF (at least with 8×3 ml, until the supernatant was colorless). The solid was dried under vacuum, and 8.0 mg of brown solid was obtained. The solubility of the biotin-labeled nanoparticles increased dramatically to 205 mg/ml.

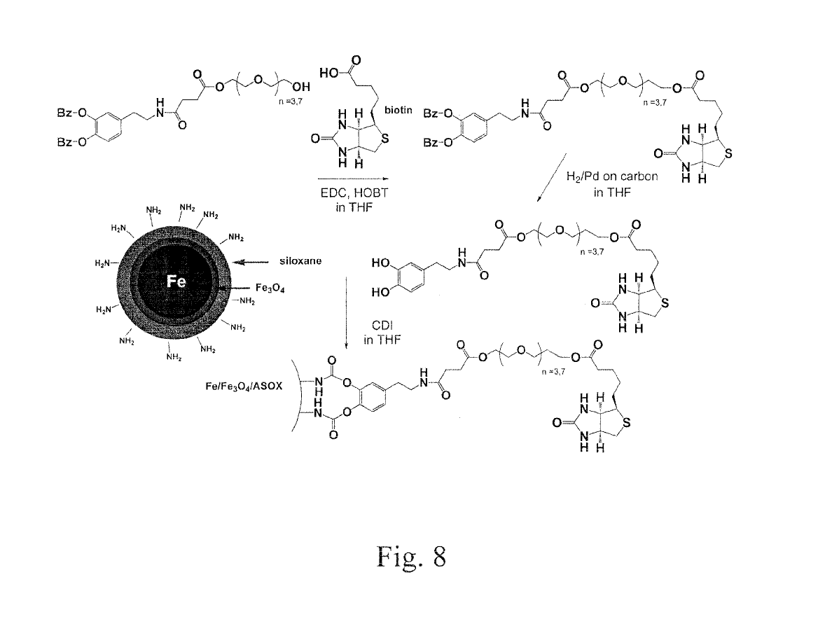

An alternative method of biotin labeling is depicted in FIG. 8 using dopamine-anchored oligoethylene glycol stealth ligands, and Fe/Fe₃O₄-ASOX nanoparticles. The free aliphatic hydroxyl group on the ligand permits the attachment of a biotin label by means of an ester bond using well-established EDC chemistry. (EDC: 1-ethyl-3-(3-dim ethyl aminopropyl) carbodiimide, HOBT: 1-hydroxybenzo-triazole, CDI: 1,1-carbonyldiimidazole).

### Example 8

### Alternative Nanoplatform Assembly Method A

In this Example, a nanoparticle-nanoparticle assembly was prepared by first connecting dopamine anchors to a protease consensus sequence. The dopamine anchor was then used to bind two nanoparticles together, followed by coating the remaining surface of the nanoparticle with dopamine-anchored (monodendate) ligands.

A. Acid chloride Ligand Stock Solution

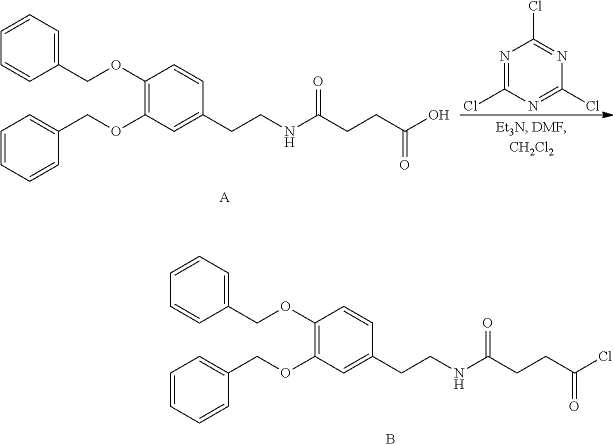

First, 50 mg of benzyl-protected dopamine-based anchor A was dissolved in 5 ml methylene chloride. Next, 21.3 mg (1 equiv.) of cyanuric chloride, 1 equiv. of Et₃N, and 2 mg of DMF were added to the solution. After stirring at room temperature for 3 hours, a white precipitate came out. The precipitate was removed by filtering through a short pad of pre-dried celite and the filtrate was concentrated under vacuum to give 48 mg of white solid. Then, 20 ml of dry THF was added to dissolve the solid to make a stock solution.

B. Linking with Cleavage Sequence

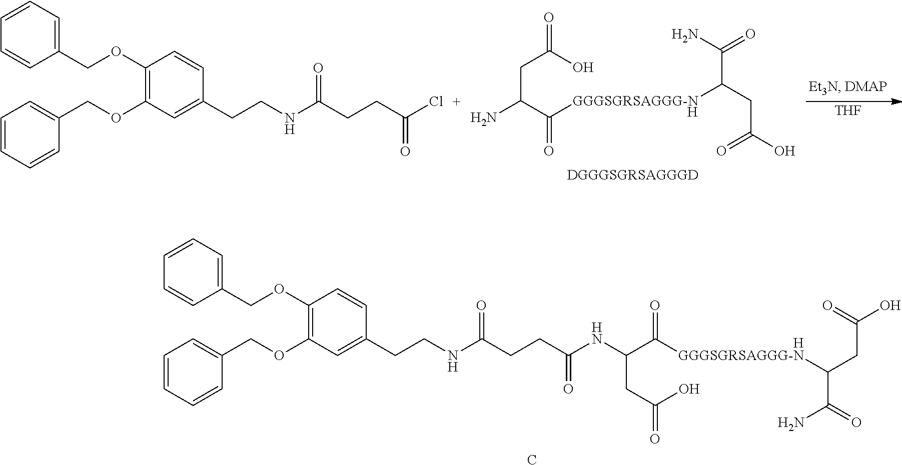

Next, 5.6 mg of the target protease cleavage sequence (DGGGSGRSAGGGD, SEQ ID NO: 65) was dissolved in 5 ml dry THF, followed by the addition of 1 ml of the dopamine anchor acid chloride stock solution (made in the previous step), along with 1 mg Et₃N and 1 mg DMAP. The solution was stirred at room temperature for 12 hours. The solvent was then removed under vacuum. After washing the residue with ether (3×3 ml), 4.6 mg of off-white solid were obtained. MS-ESI⁻: m/z 1,463.7. Molecular weight: 1,462.7.

### C. Addition of Second Benzyl-Protected Dopamine-Based Anchor

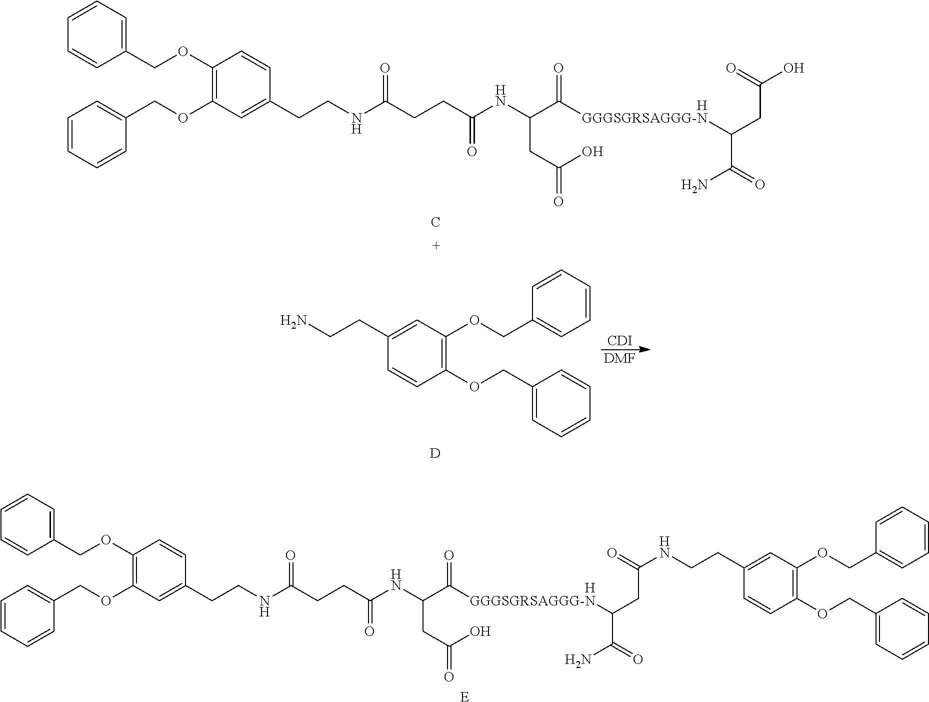

Then, 4.6 mg of product C was dissolved in 3 ml of dry DMF, followed by the addition of 0.6 mg (1 equiv.) CDI. The solution was stirred at room temperature for 30 minutes. Next, 1.2 mg (1.1 equiv.) of dopamine-based anchor D was added. The solution was stirred at room temperature for 6 hours, at which point TLC showed most of D disappeared. The solution was poured into 20 ml of ether and the organic phase was washed with cold 1N HCl (3×2 ml), cold water (3×2 ml) and brine (1×2 ml). After drying over anhydrous MgSO₄, solvent was removed under vacuum, and 3.1 mg of solid E were obtained.

### D. Debenzylation

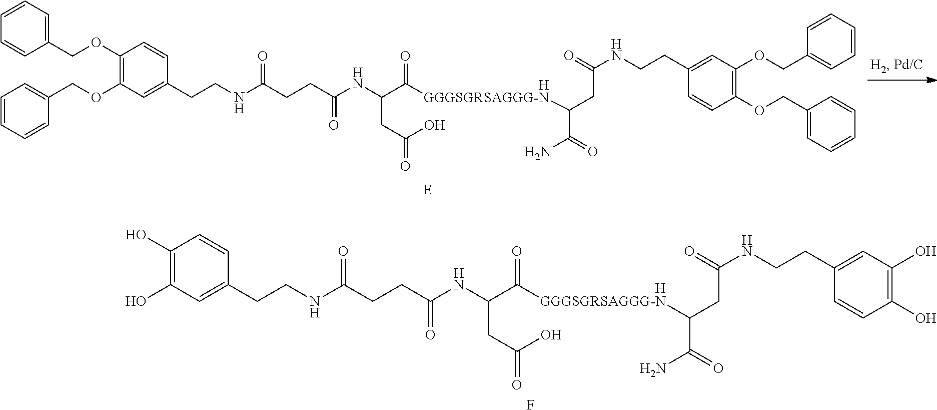

3.1 mg of product E was dissolved in 5 ml of methanol, followed by the addition of 3 mg 10% Pd/C. The system was subjected to 1 atm. H₂ atmosphere for 12 hours while stirring. The catalyst was removed by filtering through a fine filter paper. 2.3 mg clear oil F were obtained after removing solvent.

### E. Nanoparticle Assembly

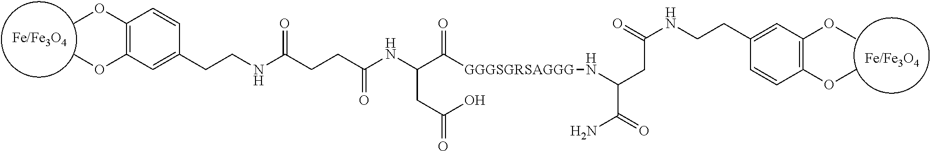

Finally, 2.3 mg of linked dopamine based anchors F were dissolved in 5 ml THF, followed by the addition of 3 mg Fe/Fe₃O₄ nanoparticles (NanoScale Corporation). The suspension was sonicated at room temperature for 1 hour, and the nanoparticles were collected by a strong magnet, and washed with THF (5×3 ml). After drying under vacuum for 2 hours, 2.2 mg of linked nanoparticles were obtained. The remaining surface of the nanoparticle can then be coated with ligands. Alternatively, the nanoparticle may already be stealth protected prior to attachment of linked dopamine anchors, or have a siloxane protecting layer.

### Example 9

### Alternative Assembly Method B

In this procedure, four target protease consensus sequences are linked to a tetracarboxylphenyl porphyrin (TCPP). The other end the cleavage sequences are linked to the glycine tips of two stealth-coated Fe/Fe₃O₄ or Fe/Fe₃O₄/ASOx nanoparticles.

### A. Acid Solution

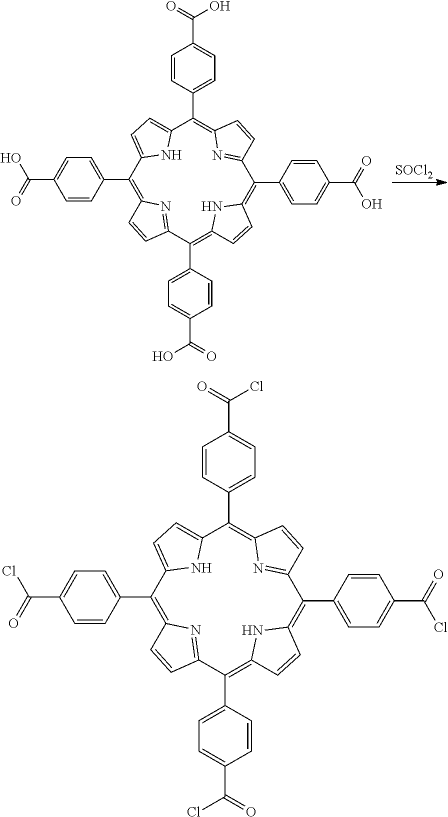

First, 6 mg of porphyrin (TPP-COOH) was dissolved in 3 ml thionyl chloride. The solution was refluxed for 2 hours at 85° C. The excess thionyl chloride was removed under vacuum. The solid was further dried under high vacuum for 6 hours.

### B. Porphyrin-Cleavage Sequence Attachment

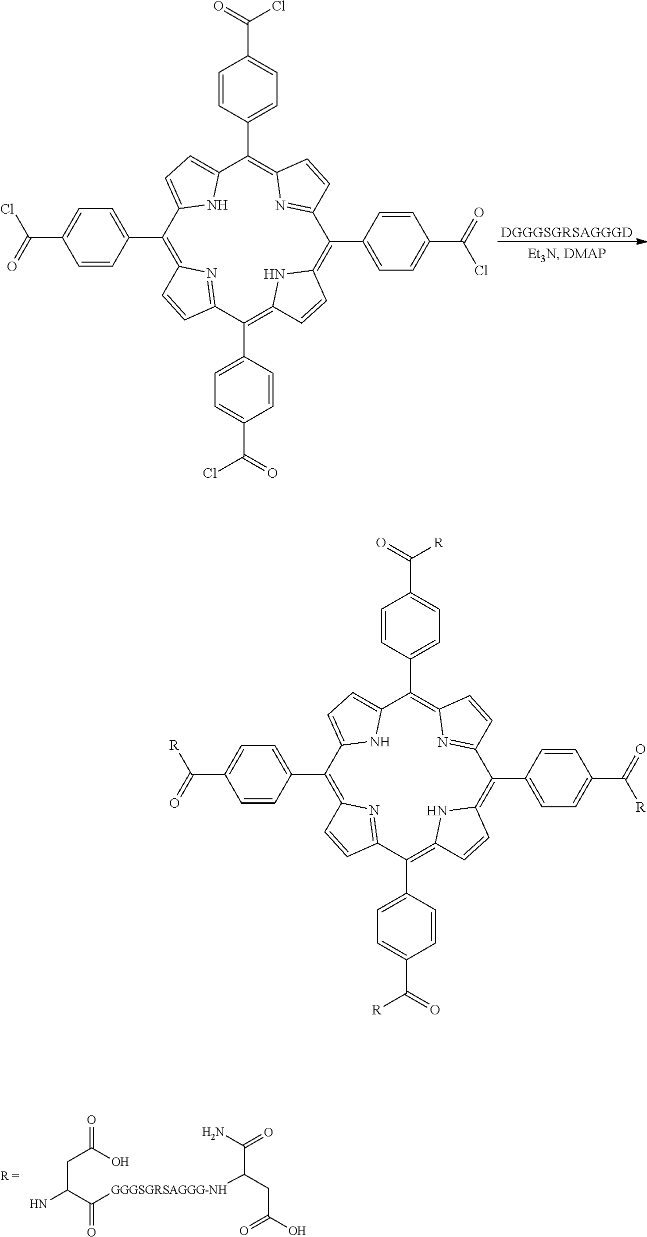

After dissolving the solid in 5 ml dry DMF, 32 mg (4 equiv.) of cleavage sequence (DGGGSGRSAGGGD; SEQ ID NO: 65) was added, followed by 0.05 ml Et₃N and 2 mg DMAP. The solution was stirred at room temperature for 18 hours. Mass spectrum showed the disappearance of starting materials and the di-peptide sequence coupled porphyrin. MS-ESI⁻: m/z 2,884.3. Molecular weight: 2,883.3.

### C. Stealth-Coated Nanoparticles

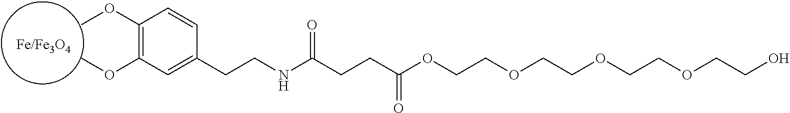

Stealth-coated nanoparticles were prepared by suspending 8 mg of Fe/Fe₃O₄ nano particles in 5 ml THF, followed by the addition of 20 mg of dopamine-based tetraethylene glycol ligand. The mixture was sonicated for 60 minutes. The nanoparticles were then collected by a strong magnet, and the excess ligand was washed away by THF (5×3 ml).

### D. Porphyrin Attachment

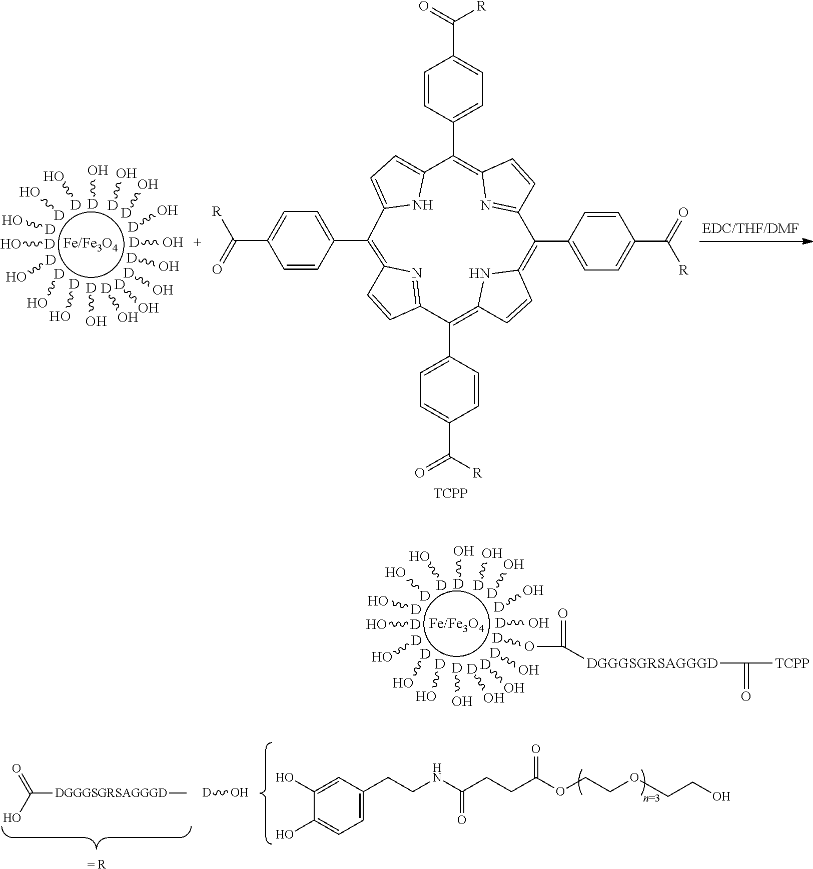

The dopamine tetraethylene glycol-modified (i.e., stealth coated) Fe/Fe₃O₄ nanoparticles were suspended in 5 ml THF, followed by the addition of 1 ml of the porphyrin tethered cleavage sequence DMF solution and 6 mg of EDC were added. The mixture was sonicated at room temperature for 60 minutes. The nanoparticles were collected by a magnet again, and washed with THF (10×3 ml). 6.2 mg of porphyrin linked stealth-coated nanoparticles were obtained after drying under vacuum.

### Example 10

### Alternative Method of Stealth Ligand Linking

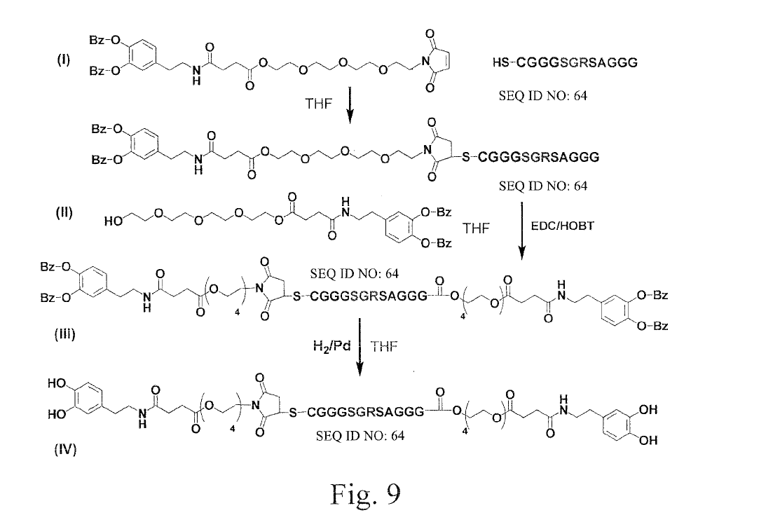

In this Example, two dopamine-based ligands were linked according to the reaction scheme in FIG. 9. Starting ligand (I) readily reacts with the thiol group of the terminal cysteine of the cleavage sequence for urokinase. Other cleavage sequences would be linked via their terminal cysteine groups as well. The glycine will be connected via an ester bond to the alcohol function of the second ligand (II) using well-established EDC/HOBT chemistry. The ligands can then be deprotected in one step with hydrogen/palladium on carbon, as previously described.

### Example 11

### Measurement of NMR Relaxation Times

The influence of various concentrations of the inventive Fe/Fe₃O₄ nanoparticle MRI contrast agents on the T₁- and T₂-relaxation behavior of ¹H-spins in water were determined using a 400 MHz NMR (Varian, field strength 9.4 T). Nanoparticles stabilized with tetraethyleneglycol ligands, and non-stealth coated nanoparticles were used. The stealth coated nanoparticles featured chemically attached porphyrins (See Example 4 above). As shown in Table IV, increasing concentrations (from 0 up to 160 μg) of Fe/Fe₃O₄ nanoparticles were suspended (non-stealth) or dissolved (stealth coated) in 1.0 ml of H₂O/D₂O (90/10 v/v). To this was added 1.0×10¹° mol urokinase (Sigma Aldrich, St. Louis, Mo.) dissolved in 0.1 ml H₂O/D₂O (90/10 v/v/). The nanoparticles were linked via a urokinase consensus sequence. The Fe core had a diameter of 5.4±1.1 nm, and the Fe₃O₄ shell had a thickness of 1.0±0.4 nm. In close proximity (d<10 nm), the magnetic spins couple and therefore, the superparamagnets strengthen each other in a magnetic Field. The measurements were conducted at 300K in standard NMR tubes. Standard T₁ and T₂ pulse sequences were used:

TABLE III
Pulse Sequences

T₁—Inversion recovery pulse sequence:
[d1]-[180]-[t]-[90]-[acquisition], where the delay, t, was varied
T₂—Carr-Purcell Meiboom-Gill (CPMGT) or spin-echo pulse sequence:
[d1]-[90]-[spin-echo]-[acquisition], where the spin-echo period is a
t-180-t block and the delay, t, was varied

TABLE IV
Pulse Sequence Results

| microgram ml⁻¹ | T₁ (A) | T₁ (B) | T₂ (A) | T₂ (B) | t (min) | r₂/r₁ |
|---|---|---|---|---|---|---|
| 0 | 0.2475 | 0.2475 | 3.565 | 3.565 | 0 | −27.6 |
| 20 | 1.157 | 2.04 | 1.717 | 0.8845 | 5 | −21.9 |
| 40 | 2.245 | 3.999 | 0.545 | 0.06156 | 10 | −19.6 |
| 60 | 2.754 | | 0.314 | 0.0652 | 15 | −16.8 |
| 80 | 3.033 | 4.055 | 0.2653 | 0.0721 | 20 | −16.5 |
| 100 | | | 0.2884 | 0.0652 | 25 | −15.8 |
| 120 | 3.172 | 4.0224 | 0.521 | 0.1253 | 30 | −15.3 |
| 140 | | | 0.751 | 0.2154 | 40 | −14.8 |
| 160 | 3.239 | 3.985 | 2.121 | 1.77 | 50 | −14.1 |
| | | | | | 60 | −13.5 |

The field strength used was higher than in clinical MRI's, however, the data obtained at higher fields are very comparable to the lifetimes in clinical MRI applications.

The stealth ligand-coated Fe/Fe₃O₄ nanoparticles achieved T₁ relaxivity of r₁=150±20 mM s⁻¹ and a T₂ relaxivity of r₂=−4300±250 mM and r₂/r₁=−28, which is advantageous in T₁-enhancement, T₂-decrease and the ratio or r₂ and r₁ compared to existing MRI contrast agents. According to the results from previously reported Monte-Carlo simulations, the coupled Fe/Fe₃O₄ nanoparticles influence the T₂-relaxation of the surrounding ¹H-spins similar to a nanoparticle of their combined radii. In the presence of urokinase, the specific consensus cleavage sequence (SGRSA, SEQ ID NO: 2) of the linker will be cut and, therefore, the Fe/Fe₃O₄ nanoparticles become separated. Consequently, they now decrease T₂ relaxation time to a lesser extent.

After the protease-cleavage of the linker, r₁ increased slightly to 180±20 mM s⁻¹, whereas r₂ increased to −2,350±250 mM s⁻¹, with the r₂/r₁ ratio being −13. The remarkable change in T₂ combined with an almost constant value for T₁ permits the spatially-resolved in-situ measurement of the protease activity in the mammalian body by comparing T₁- and T₂-weighted MRI images at various times.

The results are depicted in FIGS. 10-11. Line A is the non-stealth ligand-coated nanoparticle. Line B is the stealth ligand coated nanoparticle. FIG. 10 indicates that both the non-stabilized and the tetraethylene glycol stabilized bimetallic nanoparticles increase the T₁ relaxation time. The presence of the tetraethylene glycol layer did not hamper the magnetic effects of the nanoparticle on the surrounding H₂O/D₂O mixture. This is a clear advantage of the Fe/Fe₃O₄ nanoparticles, as compared with gadolinium-based contrast agents. The maximally observed T₁ increase was 16 times, which is close to the best results reported in the art.

FIG. 11 shows a remarkable decrease in T₂ (up to a factor of 57) when the Fe/Fe₃O₄-nanoparticles are added. The observed significant decrease in T₂ demonstrates that the nanoparticles can be used as MRI contrast agents. The presence of the tetra(ethylene glycol) ligands leads to an even more significant decrease of T₂, as shown by line B. T₂ increased for both particles once the nanoparticle concentration reached 120 μg/ml.

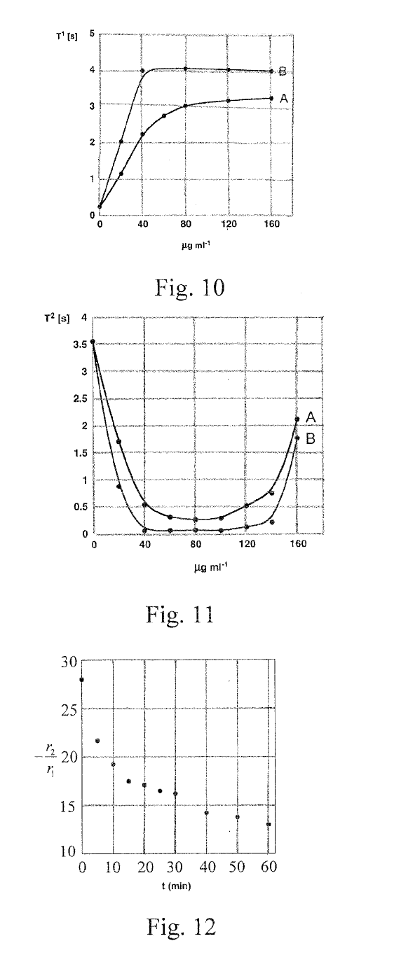

FIG. 12 illustrates the decrease of −(r₂/r₁) over time as linked nanoparticles are cleaved by urokinase. For this measurement, 40 μg of porphyrin-labeled stealth coated Fe/Fe₃O₄ nanoparticles linked by a cleavage sequence for urokinase (DGAGSGRSAGAGD, SEQ ID NO: 66) were dissolved in 0.9 ml H₂O/D₂O at 300K. To this was added 1.0×10⁻¹⁰ mol urokinase (Sigma Aldrich, St. Louis, Mo.) dissolved in 0.1 ml H₂O/D₂O (90/10 v/v/). The measurements were conducted at 300K using standard pulse sequences for T₁ and T₂ measurements at 400 MHz. The r₁ and r₂ values were then calculated and plotted on the graph in FIG. 12.

### Example 12

### FRET Based Assays

The fluorescence of free sodium tetracarboxylate porphyrin (at pH=6.8 in PBS) and zinc-doped sodium tetracarboxylate porphyrin was studied, and results compared with those obtained for core/shell Fe/Fe₃O₄-nanoparticles to (NanoScale Corporation; Manhattan, Kans.) nanoparticles featuring stealth ligands with chemically-attached metalated and unmetalated tetracarboxyphenyl porphyrin (TCPP).

First, both the “free” sodium tetra-carboxylate porphyrin and the zinc-doped sodium tetracarboxylate porphyrin are tethered to Fe/Fe₃O₄-nanoparticles. To prepare the stealth-protected Fe/Fe₃O₄-nanoparticles, 35 mg of dopamine-tetraethylene glycol ligand were dissolved in 5 ml THF. Next, 11.0 mg of Fe/Fe₃O₄-nanoparticles were added and sonicated at room temperature for 1 hour. The core of the nanoparticles had a diameter of from about 3-5 nm. The Fe₃O₄ shell had a thickness of less than 2 nm. The solid was then collected with a magnet and solvent was decanted carefully. The solid was washed with THF (3×3 ml). After drying under vacuum for 2 hours, 10.0 mg of stealth-protected nanoparticle product was obtained.

The oligopeptide linker was then attached to the metalated porphyrin. First, 5.0 mg of the porphyrin was refluxed in 5.0 ml SOCl₂ at 100° C. for 30 minutes. The excess SOCl₂ was then removed under high vacuum, and the resulting solid was further dried under vacuum for 3 hours. Next, 4 mg of the oligopeptide sequence and 5 ml THF were added to the porphyrin solid and stirred at room temperature for 5 hours. The THF was then removed under vacuum, and a greenish-colored solid was obtained. Electrospray ionization (ESI) mass spectrometry showed a mixture of at least 2 linked porphyrin species (mono-peptide and di-peptide linked to porphyrin). The same procedure was used to attach the oligopeptide linker to the non-metalated porphyrin.

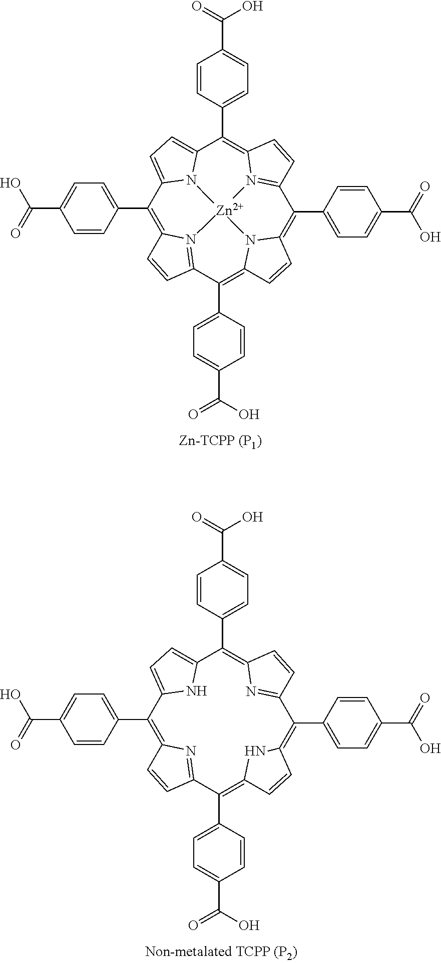

To attach the porphyrins to the nanoparticles, the metalated porphyrin-oligopeptide solid was dissolved in 10 ml dry THF. Next, 5.0 ml of this solution was added to 10.0 mg of the dopamine tetraethylene glycol-tethered Fe/Fe₃O₄ nanoparticles, followed by 1.0 mg 4-dimethylaminopyridine (DMAP) and 8.0 mg EDC. The resulting suspension was sonicated for 1 hour at room temperature. The solid precipitate was collected by magnet and thoroughly washed with THF (8×2 ml). The sample was then dried under high vacuum for 5 hours. 8.0 mg of product was obtained. The procedure was repeated to attach the non-metalated porphyrin to the nanoparticle.

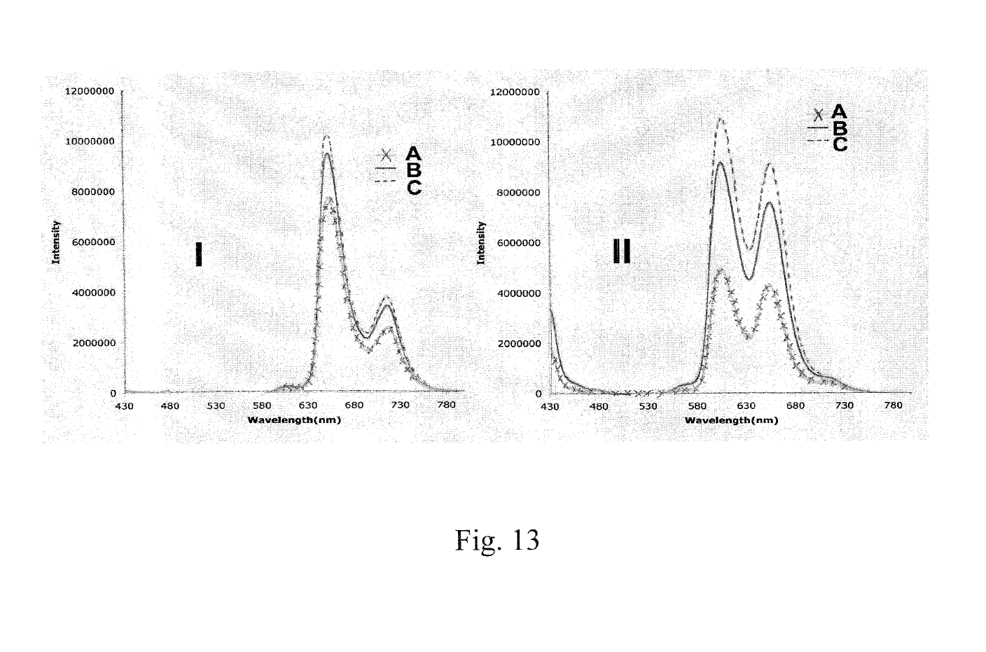

As shown in FIG. 13, for both tethered porphyrins, the emission intensity rises slightly less than linear with increasing concentration of the nanoplatforms. This is a first indication of Forster energy transfer (FRET), as discussed below. The number of porphyrins that are tethered to one Fe/Fe₃O₄-nanoparticle (d=20 nm) in FIG. 13 was estimated to be 4.8 (I) and 4.5 (II).

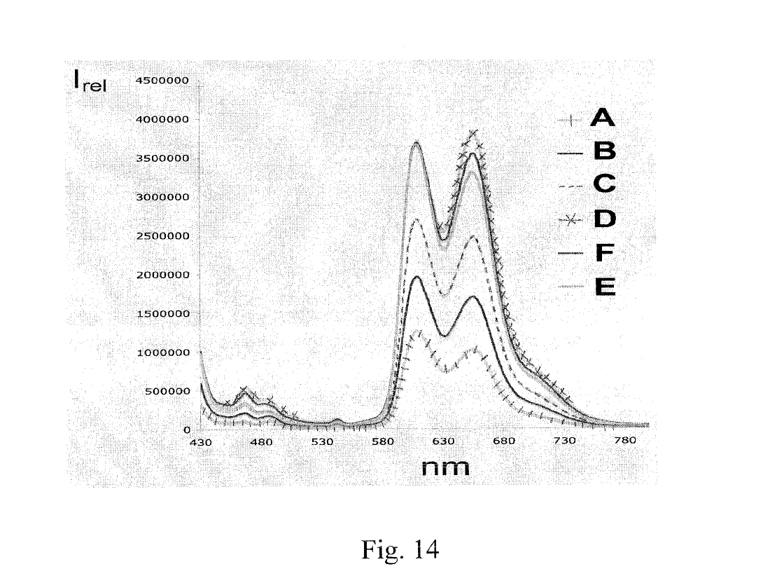

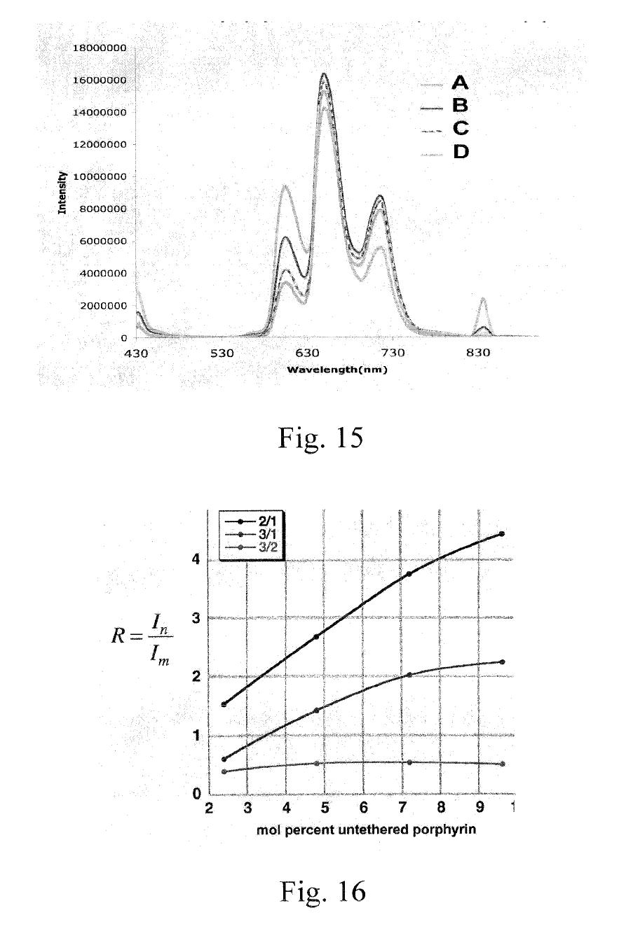

FIG. 14 shows the concentration dependence of zinc-doped sodium tetracarboxylate porphyrin and sodium tetracarboxylate porphyrin, in a relative molar ratio of 9 to 1, in PBS. Whereas the first fluorescence band at λ=609 nm shows saturation, the second band at λ=657 nm shows a maximum of intensity at the concentration of c=8.0×10⁻⁶M nanoplatforms. As the concentration increases, Förster energy transfer (FRET) increases: the hopping of excited states from porphyrin to porphyrin increases the degree of internal (radiation-less) conversion. So, the fluorescence quantum yield does not exceed a maximum of F=0.011 for the Fe/Fe₃O₄-bound porphyrins. The emissions from the zinc-doped sodium tetracarboxylate porphyrin=607 nm, λ₂=657 nm) are higher in energy than those of the “free” sodium tetracarboxylate porphyrin (λ₁=654 nm, λ₂=718 nm). Therefore, FRET is directed towards the “free” porphyrin, which shows a slight relative emission enhancement (f<2.2 from the analysis of the spectra shown in FIG. 15 when bound to Fe/Fe₃O₄ nanoparticles). The number of porphyrins tethered to one Fe/Fe₃O₄-nanoparticle (d=20 nm) in FIG. 14 is estimated to be 52.

The emission spectra of the nanoplatform assembly (1×10⁻⁵ M) in PBS in the presence of about 1×10⁻⁸ M urokinase is depicted in FIG. 15. Untethered sodium tetracarboxylate porphyrin was added to the Fe/Fe₃O₄ nanoplatform featuring zinc-doped sodium tetracarboxylate porphyrin and sodium tetracarboxylate porphyrin in a relative molar ratio of 9 to 1 in PBS. A: c=2.8×10⁻⁶ M added porphyrin, B: c=5.6×10⁻⁷ M added porphyrin, C: c=8.4×10⁻⁷ M added porphyrin, D: c=1.2×10⁻⁷M added porphyrin. A distinct decrease of the fluorescence band is visible at λ₁=607 nm. The concentration dependence of the fluorescence occurring from the other two fluorescence bands at (λ₂=654 nm, λ₃=718 nm) is non-linear. The reason for the observed non-linear behavior can be found in the high fluorescence quantum yield of the non-metalated, untethered sodium tetracarboxylate porphyrin. We estimated Φ=0.082, which is approximately eight times higher than in the tethered state, when the large porphyrin-concentration in the sphere around the Fe/Fe₃O₄ nanoparticle leads to increased FRET and, consequently, radiation-less deactivation of the excited states.

In FIG. 16, the ratios of the integrals of the fluorescence bands shown at λ₁=607 nm, 654 nm and λ₃=718 nm are plotted versus the mole percent of added untethered sodium tetracarboxylate porphyrin (as measured by HPLC using an Agilent workstation (HP 1050) equipped with an optical detection system). The plots of R=I(λ₂)/I(λ₁) and R=I(λ₂)/I(λ₁) increase with increasing mol percent of added untethered porphyrin. They are quite linear in the concentration range from 0 to 7 mol percent of added untethered sodium tetracarboxylate porphyrin. Therefore, the concentration of porphyrin that is “freed” by the enzyme urokinase, which will be cleaving the urokinase-cleavage sequence (SRGSA, SEQ ID NO: 2), can be measured by recording fluorescence spectra of the nanoplatform at different time intervals and comparing the fluorescence intensities at the three wavelengths. All three wavelengths permit in vivo-measurements in mammalian tissue, especially when coupled with single-photon counting techniques (fluorescence microscopy).

### Example 13

### In Vitro Urokinase Sensor

In this Example, TCPP was tethered via an oligopeptide containing a urokinase-specific cleavage sequence (SGRSA, SEQ ID NO: 2) to a dopamine-tetraethylene glycol ligand. This ligand was then bound to the Fe/Fe₃O₄-nanoparticles. The assembly is prepared using the same procedures described above in Example 12, except that only one type of porphyrin was used (i.e., non-metalated only or metalated only).

Although the plasmon band of the inner Fe core did not appear in the UV/Vis spectrum due its small diameter, it was able to quench the luminescence occurring from TCPP. This type of sensor is based on the quenching of the excited states of chromophores (e.g. porphyrins) with organic (e.g. viologens) or inorganic quenchers (e.g. metal, alloy, and core/shell nanoparticles). Due to the proximity of the nanoparticle (˜2 nm) to the porphyrin, the surface plasmon of the core/shell nanoparticle is able to quench the emission spectra from the chemically-attached porphyrin. Once released by urokinase cleavage, the luminescence increases significantly. This luminescence increase can be detected spectrally. When several chromophores featuring discernible emission spectra are used, the activity of various enzymes can be detected simultaneously.

The light-switch mechanism was tested using 3 samples of urine from rats impregnated with MATB III type cancer cells (rodent model for aggressive breast cancer), since urokinase can pass the mammalian kidneys and retains at least some activity in urine. The samples were collected 5 days (control) and 36 days after cancer impregnation, respectively, and immediately frozen at −80° C. Before testing, the urine samples were thawed and heated to 37° C. The following procedure was used to test each sample.

The TCPP-nanoparticle nanoplatform assembly was dissolved in bidest. water using sonication for 30 minutes. Next, 100 μl of urine was added to a 5×10⁸ M solution of the nanoplatform assembly in water. The temperature was kept constant at 34° C. The fluorescence spectra was recorded every 2 minutes.

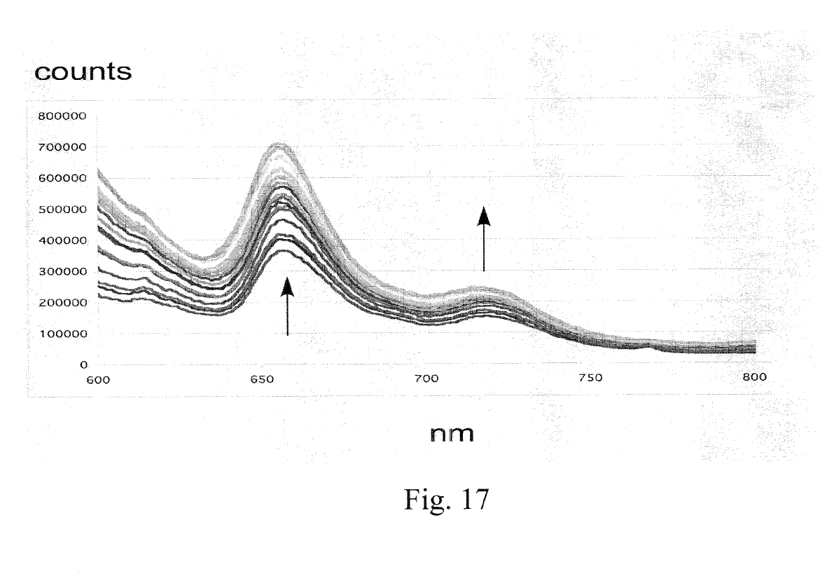

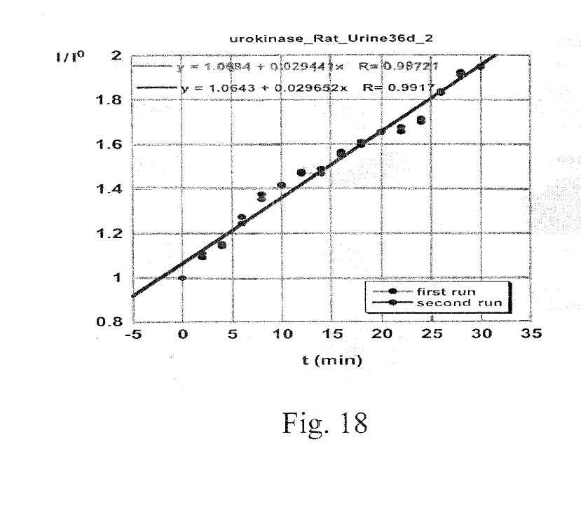

As can be seen from FIG. 17, the luminescence from TCPP increased steadily over time for the 36 day urine. The control (5 day urine) did not demonstrate a significant increase in luminescence. FIG. 10 shows the plot of the relative intensities of the luminescence of TCPP occurring at λ=656 nm using the measurement shown in FIG. 17. The assay was tested twice using the 36 day urine, and the measurements in FIG. 18 show that it was highly reproducible.

### Example 14

### In Vivo Urokinase Assay

An in-vivo urokinase-assay was tested in Charles River female mice, which have been impregnated with B16F19 mouse melanoma cells 10 days prior to these measurements. The mice were anesthetized and then a solution of a Fe/Fe₃O₄-nanoparticle-TCPP assembly was administered to the mice intravenously (IV) or via direct injection into the tumors (IT). The IV solution was 200 μg of the nanoparticle assembly in 200 ml PBS. The IT solution was 100 μg of the nanoparticle assembly in 200 ml PBS. To measure the activity of the assay, the mice were anesthetized again and placed under a fluorescence microscope employing a single-photo-counting detector. This instrument has been built in-house. The tumor regions at the hind legs of the mice were excited using laser light (Ti:sapphire-laser, λ=870 nm, P=6.5 mW) in the IR-region.

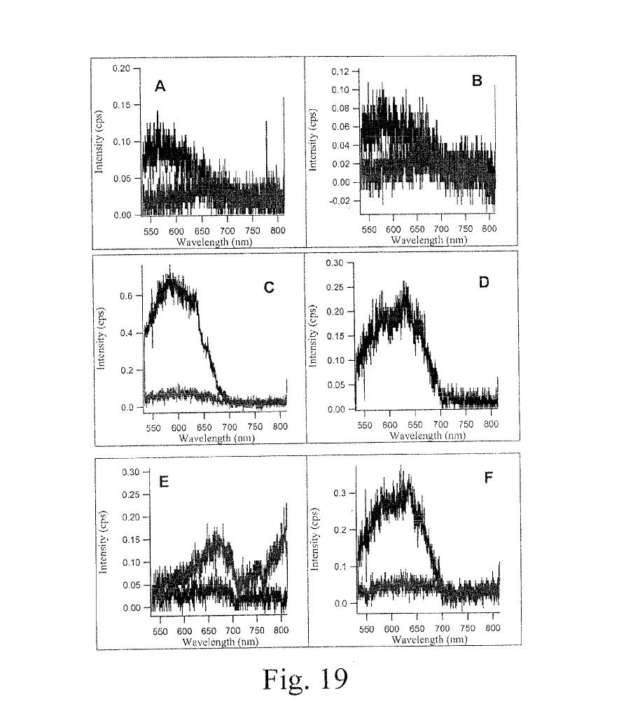

The results of the single-photo-counting spectra, from the right and left limbs of the mice, recorded through a fluorescence microscope (resolution: 1 m×1 m×1 m) is illustrated in FIG. 19 (red: left limb; blue: right limb). Box A shows the results from mouse 1, which was IT-injected 30 minutes prior to measurement. Box B shows the results from mouse 2 (no tumors), which was IV-injected 12 hours prior to measurement. Box C shows the results from mouse 3 (bearing tumors on both legs), which was IV-injected 12 hours prior to measurement, Box D shows the results of mouse 4, which was IV-injected 24 hours prior to measurement. Box E shows the results from the control mouse, neither IT- nor IV-injected. Box F is a repeat of C from mouse 7.

The porphyrin, TCPP, requires tri-photonic excitation at this excitation wavelength. It is remarkable that the signal strengths obtained in the right legs of the tumor-bearing mice correlates with the tumor size, whereas the signal in the left limb apparently does not. The hypothesized explanation is that the uptake of the nanoparticle assembly by the tumors is so rapid, that the first tumor, which is encountered by the nanoparticles injected intravenously, incorporates almost everything. It was found that the IT-injection is less efficient than IV-injection, because the urokinase does not have the time to cleave the majority of the cleavage sequences and the porphyrin does not light up.

### Example 15

### Nanoparticle-Porphyrin Assemblies

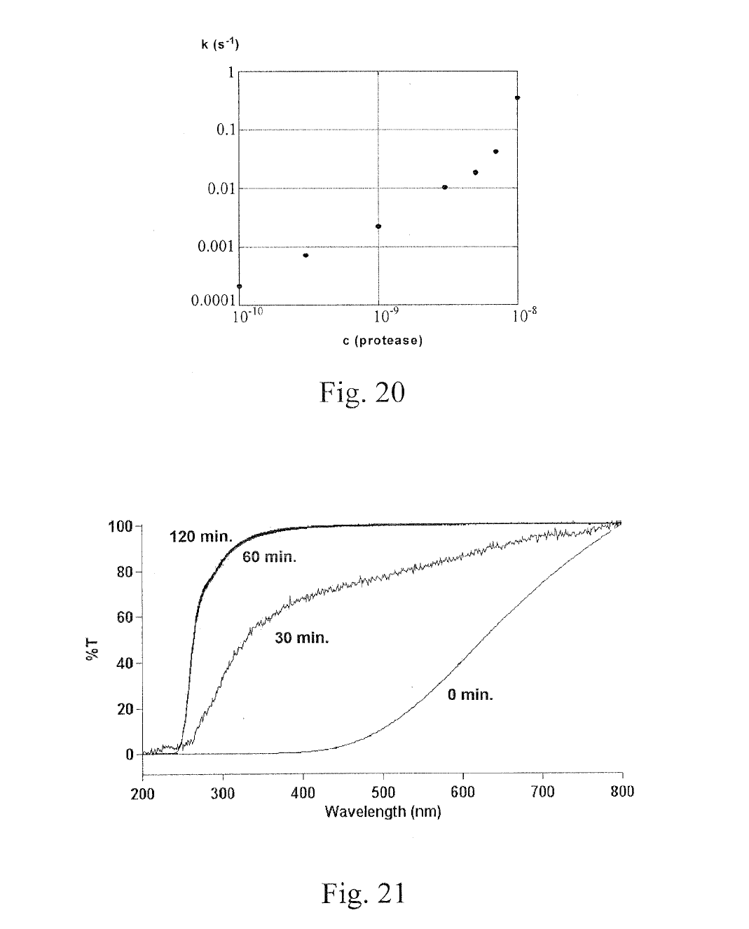

In this Example, stealth-protected. Fe₃O₄ nanoparticles were linked to one or more organic chlorins and/or phthalocyanines via target protease consensus sequences. The luminophores feature distinct emission spectrums in the region between 650 and 900 nm. Charles River mice bearing B16F10 melanomas were intravenously injected with 100 μg of the nanoparticle assay in PBS. The targeted area was then excited using a Ti:sapphire laser at wavelengths ranging between 800 and 1,050 nm. Once the nanoplatform is in the vicinity of the cancerous tissue, the linkage is cleaved by the proteases. This stops the quenching of the luminescence by the nanoparticle, and the luminophore lights up. The intensity of the light is directly correlated to the level of enzyme activity. In addition, a positive correlation was found between tumor size and the intensity of the emitted light. This mechanism could be used as a visual reference for locating tumors, and as a luminescent contrast enhancer during tumor removal surgery. FIG. 20 shows the typically observed protease cleavage kinetics as a function of protease (urokinase) concentration, at a pH 6.8 and temperature of 36° C.

### Example 16

### Light Backscattering Sensor

In this Example, a UV/Vis-spectrometer was used to measure the activity of uPA in two different experiments.

A first nanoplatform was prepared using Fe/Fe₃O₄ nanoplatforms linked via a urokinase consensus sequence (DGGSGRSAGGGC, SEQ ID NO: 68). The nanoplatforms included a ligand stealth coating and attached porphyrin. The solution was prepared by dissolving 0.010 mg of the linked nanoplatforms in 3.0 ml phosphate buffer (pH=6.8) containing 100 ml of rat urine from rats with advanced pancreatic cancer (estimated concentration of urokinase: 5×10⁻¹⁰ M). The assay was then excited using a light beam. The change in the optical properties is clearly discernible upon the cleavage of the oligopeptides-linker by urokinase. The UV/Vis backscattering spectrum of a nanoparticle-dimer is shown in FIG. 21 over a period of 120 minutes.

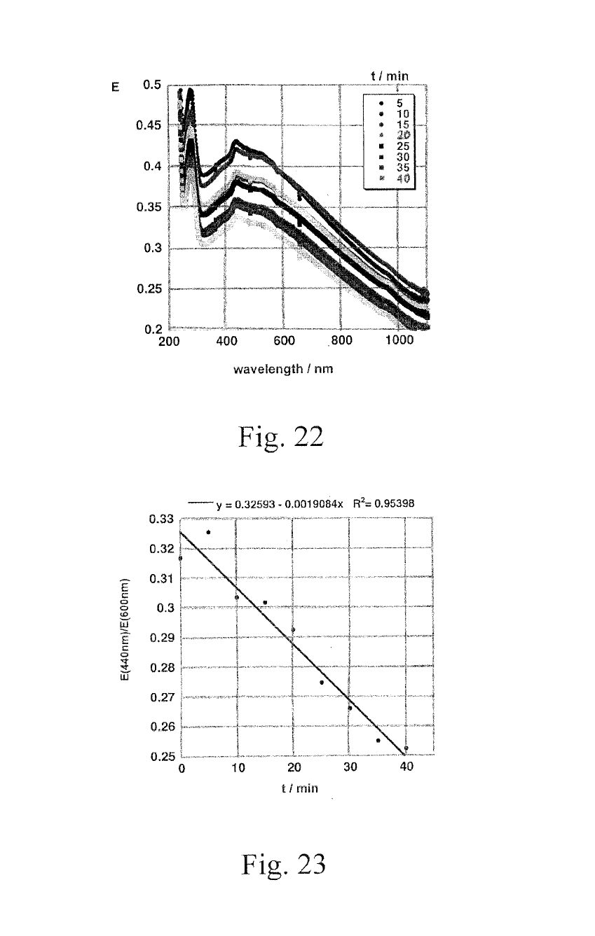

A second nanoplatform assembly was prepared according to Example 9 using a TCPP-tether. 1.0 mg of the nanoplatforms were dissolved in 3.0 ml of aqueous buffer (0.01M PBS). The temperature was kept constant at 36.8° C. Next, the urokinase was added to the aqueous PBS mixture at a concentration of 1×10⁻¹⁰ M. The assay was then excited using a light beam. The UV/Vis-spectrometer recorded the optical extinction E=absorption (A)+scattering (S), at t=0, 5, 10, 15, 20, 25, 30, 35, and 40 minutes. It was assumed that the absorption spectrum does not change during 45 min, as a control measurement taken without urokinase has shown. Therefore, the observable change of the extinction is caused by the change in scattering once the oligopeptide-tether is cleaved by the enzyme. FIG. 22 shows the changes in extinction during a period of 40 min.

To visualize the kinetics of reaction, the signal intensity at 440 nm, divided by the signal intensity at 600 nm was plotted vs. the progress of time. As FIG. 23 indicates, a linear slope has been obtained. The observed kinetics permit an estimate of the amount of protease in the tissue. That is, the speed of cleavage is directly related to the concentration of urokinase, and thus, the speed of cleavage can be correlated with the aggressiveness of the tumor.

### Example 17

### Photophysical Properties of Fe/Fe₃O₄ Nanoparticle Assemblies

Fe/Fe₃O₄-nanoparticles were stabilized using Ligands 1-3, with ligands 2-3 featuring chemically attached porphyrins. The nanoparticles had a core diameter of about 5.4 nm, and a shell thickness of about 1.5 nm.

The ligands were added to the nanoparticles in anhydrous THF (10/1 per weight with respect to the mass of Fe/Fe₃O₄) and sonicated for 5 min., then continuously stirred for 24 h. The coated bimetallic nanoparticles were then separated from the dispersion medium with a strong permanent magnet. The bimagnetic nanoparticles were then resuspended in THF, and recollected. Sonication for 30 seconds, followed by stirring for 5 min. redispersed the nanoparticles in the liquid medium. The washing/redispersion process was repeated 10 times. The residual solvent was then removed in an argon stream. Finally, the coated bimagnetic nanoparticles were suspended/dissolved in sterile deionized H₂O.

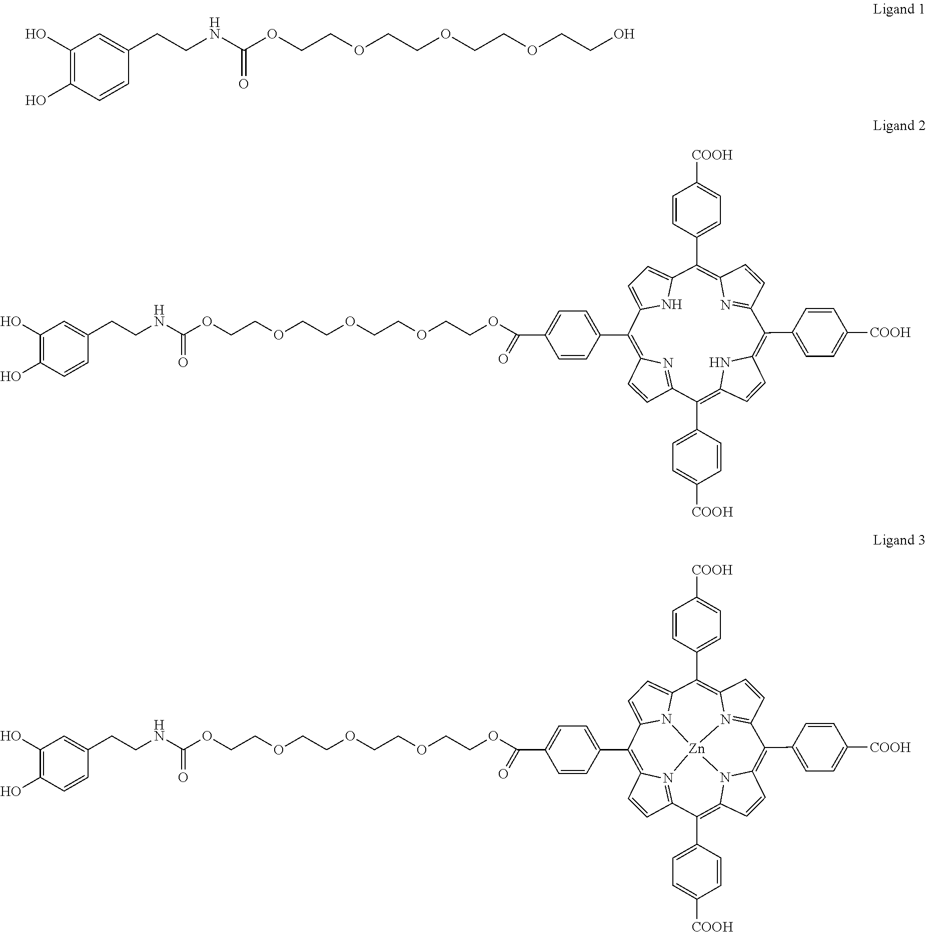

Excitation was then performed using a Ti:sapphire laser at the wavelengths indicated in Table V below. The emission was observed using a Fluoromax® 2 fluorescence spectrometer (HORIBA Jobin Yvon; Edison, N.J.). Table V shows the photophysical properties of these nanoassemblies.

TABLE V
Photophysical Properties of the Fe/Fe₃O₄/porphyrin Assemblies

| Fe/Fe₃O₄ | | | | λₑₓ | λₑₘ 1 | λₑₘ 2 |
|---|---|---|---|---|---|---|
| (nm) | L1 | L2 | L3 | (nm) | (nm) | (nm) |
| Fe (2.1 ± 0.4)/Fe₃O₄ | 0.95 | 0.05 | 0 | 417 (860)* | 654 | 720 |
| (1.1 ± 0.4) | 0.95 | 0 | 0.05 | 425 | 607 | 657 |
| Fe (5.3 ± 1.2)/Fe₃O₄ | 0.95 | 0.05 | 0 | 417 | 656 | 716 |
| (1.0 ± 0.3) | 0.95 | 0 | 0.05 | 425 | 605 | 656 |
| Fe (5.4 ± 1.1)/Fe₃O₄ | 0.95 | 0.05 | 0 | 417 | 655 | 720 |
| (1.0 ± 0.4) | 0.95 | 0 | 0.05 | 425 | 607 | 657 |

λₑₓ: Excitation wavelengths,
λₑₘ: Emission wavelengths.
*Multiphoton excitation using a Ti: sapphire laser is possible.

The phosphorescence quantum yield did not exceed a maximum of Φ=0.011 for the Fe/Fe₃O₄-bound porphyrins. Emission from the iron(0)-cores was not detectable. However, the luminescence quenching ability of the Fe/Fe₃O₄ nanoparticles was clearly discernible. The phosphorescence quantum yield of the non-nanoparticle attached porphyrins was approximately 2.2 to 2.5 times higher.

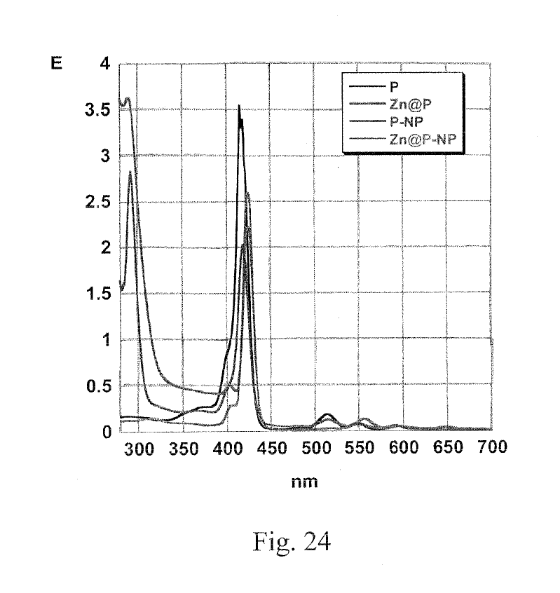

FIG. 24 shows typical UV/Vis absorption spectra of the “free” and Fe/Fe₃O₄-attached tetracarboxyphenyl porphyrin (TCPP), together with the zinc complexes of the porphyrin in H₂O at a concentration of 7.5×10⁻⁶ M. The ratio of Fe/Fe₃O₄ to porphyrin is estimated to be 1:1.2. As seen in FIG. 11, the peak positions of the Soret band (extremely intense near-ultraviolet band) are at λ=417 nm for TCPP and λ=425 nm for Zn-TCPP. The absorption coefficients are 4.8×10⁵ cm⁻¹ for TCPP and 4.1×10⁵ cm⁻¹ for Zn-TCPP, in agreement with the literature. Chemical attachment to the bimagnetic Fe/Fe₃O₄ nanoparticles via a dopamine-tetra(ethylene glycol) bridge decreases the absorption coefficient of TCPP by a factor of 2.1, whereas only a minor decrease (<1.1) is observed when attaching Zn-TCPP.

### Example 18

### Solubility and SAR Values of Nanoplatforms

In this Example, the solubility and SAR values of various nanoparticle assemblies using Ligands 1-7 was evaluated. The ligands were added to the nanoparticles (described in Tables below) in anhydrous THF (10/1 per weight with respect to the mass of Fe/Fe₃O₄) and sonicated for 5 min., then continuously stirred for 24 h. The coated bimetallic nanoparticles were then separated from the dispersion medium with a strong permanent magnet. The bimagnetic nanoparticles were then resuspended in THF, and recollected. Sonication for 30 seconds, followed by stirring for 5 min. redispersed the nanoparticles in the liquid medium. The washing/redispersion process was repeated 10 times. The residual solvent was then removed in an argon stream. Finally, the coated bimagnetic nanoparticles were suspended/dissolved in sterile deionized H₂O. Ligands 1-7 below were used.

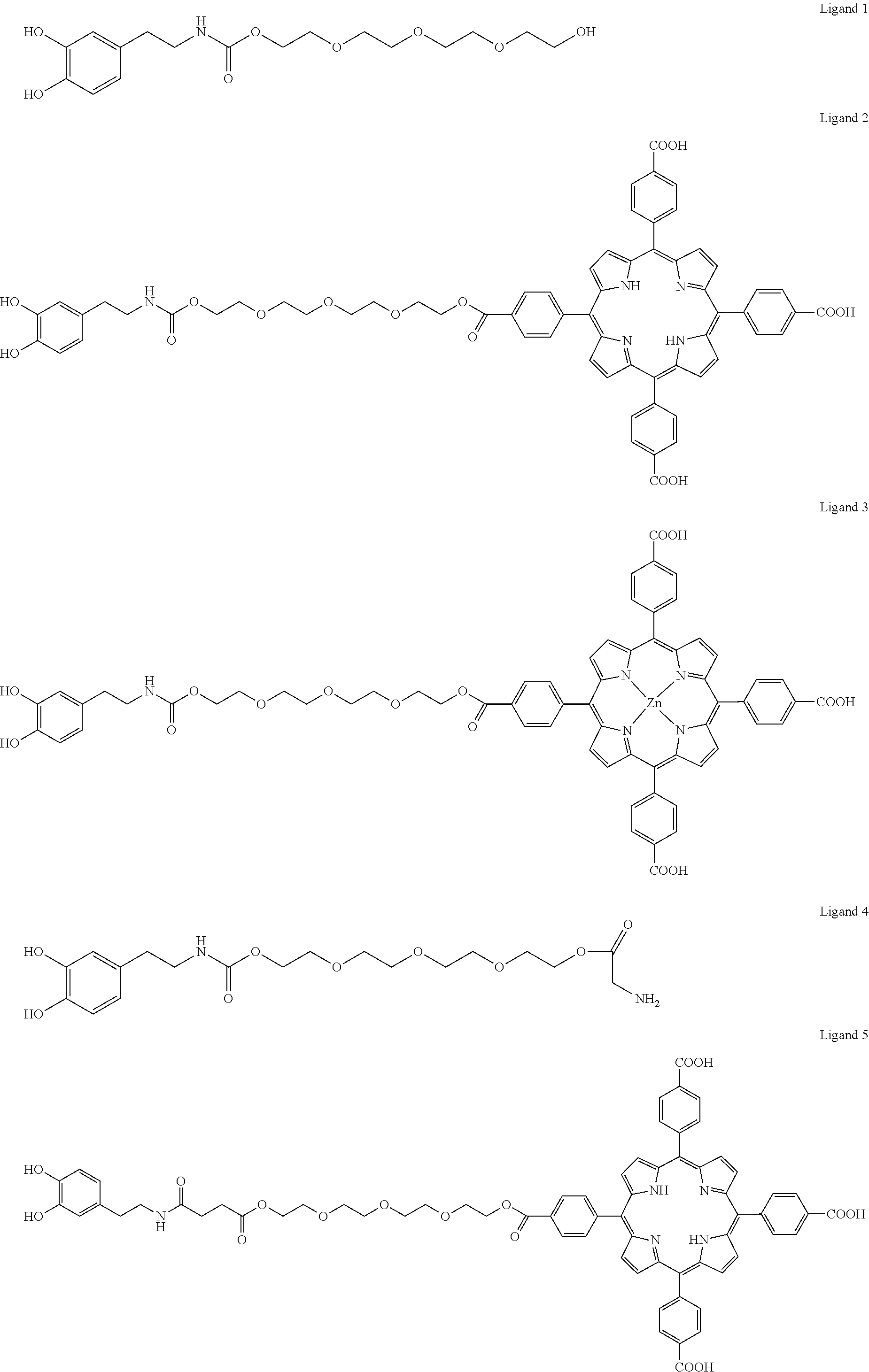

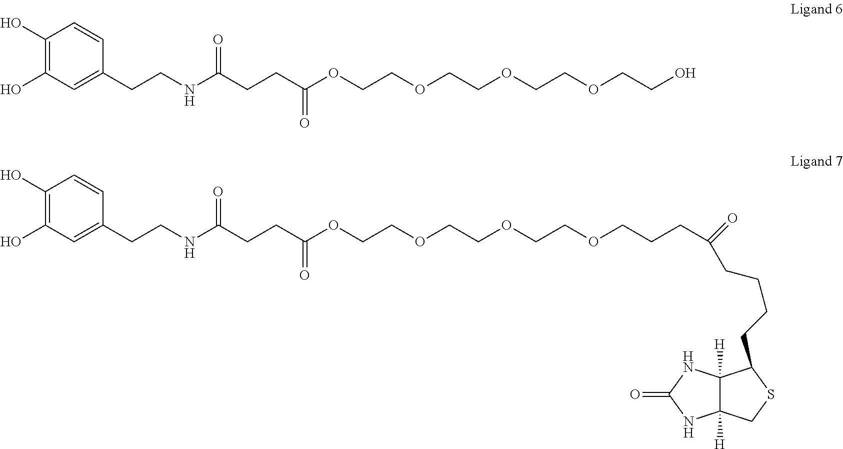

To determine solubility, phosphate buffer (0.1M, pH=6.8) was added dropwise to 0.25 mg of the nanoparticles in a glass cuvette. The suspension was continuously stirred with a micromagnetical stirrer (Fisher). The light scattering of the suspension was recorded at 700 nm. Once the particles have dissolved, the extinction (i.e., light absorption and scattering) at 700 nm decreased to less than E=0.01.

The specific absorption rate (SAR) is calculated by SAR=C*ΔT/Δt, where C is the specific heat capacity of the sample, T is the temperature, and t is the time. To determine the SAR values, the hyperthermia apparatus was developed in-house and uses a modified heavy duty induction heater converted to measure the temperature change of the sample. In the setup, a remote IR probe is used to detect the temperature change. The apparatus uses remote fiber-optic sensing and its frequency is fixed.

TABLE VI
Solubility and SAR Values of Nanoparticle-Ligand Combinations (1-4)

| | | | | | Solubility | SAR |
|---|---|---|---|---|---|---|
| Fe/Fe₃O₄ | | Ligand | Ligand | Ligand | in H₂O | (W/g |
| (nm)\hat{ } | Ligand 1 | 2 | 3 | 4 | (mg/ml) | (Fe)) |
| Fe: 2.1 ± 0.4 | 33 ± 4 | 0 | 0 | 0 | 0.015 | 25.2 |
| Fe₃O₄: | 29 ± 4 | 4 ± 3 | 0 | 0 | 0.012 | 24.8 |
| 1.1 ± 0.4 | 29 ± 4 | 0 | 4 ± 3 | 0 | 0.014 | 24.3 |
| Fe: 2.5 ± 0.5 | 1.0 | 0 | 0 | 0 | <0.005 | 56.6† |
| Fe₃O₄: | | | | | | |
| 1.0 ± 0.5 | | | | | | |
| Fe: 4.1 ± 0.3 | 35 ± 4 | 0 | 0 | 0 | 0.16 | 48.4 |
| Fe₃O₄: | 30 ± 4 | 5 ± 3 | 0 | 0 | 0.14 | 46.1 |
| 1.5 ± 0.7 | 30 ± 4 | 0 | 5 ± 3 | 0 | 0.14 | 45.3 |
| Fe: 4.5 ± 0.7 | 121 ± 11 | 0 | 0 | 0 | <0.005 | 20.0† |
| Fe₃O₄: | | | | | | |
| 2.0 ± 0.5 | | | | | | |
| Fe: 4.7 ± 0.7 | 75 ± 9 | 0 | 0 | 0 | <0.005 | 18.7† |
| Fe₃O₄: | | | | | | |
| 0.4 ± 0.1 | | | | | | |
| Fe: 5.3 ± 1.2 | 114 ± 12 | 0 | 0 | 0 | 0.11 | 48.2 |
| Fe₃O₄: | 105 ± 9 | 8 ± 6 | 0 | 0 | 0.10 | 45.7 |
| 1.0 ± 0.3 | 105 ± 9 | 0 | 8 ± 6 | 0 | 0.11 | 46.3 |
| Fe: 5.4 ± 1.1 | 118 ± 13 | 0 | 0 | 0 | 0.075 | 47.4 |
| Fe₃O₄: | 108 ± 10 | 9 ± 6 | 0 | 0 | 0.065 | 46.6 |
| 1.0 ± 0.4 | 108 ± 10 | 0 | 9 ± 6 | 0 | 0.068 | 48.1 |
| | 108 ± 10 | 8 ± 6 | 4 ± 3 | 0 | 0.070 | 46.5 |
| | 95 ± 10 | 0 | 0 | 25 ± 7 | 0.35 | 43.2 |
| | 88 ± 8 | 9 ± 6 | 0 | 25 ± 7 | 0.34 | 43.4 |
| Fe: | 88 ± 8 | 0 | 9 ± 6 | 25 ± 7 | 0.35 | 63.1 |
| 5.4 ± 1.1* | 88 ± 8 | 8 ± 6 | 3 ± 2 | 25 ± 7 | 0.35 | 63.3 |
| Fe₃O₄: | 108 ± 10 | 10 ± 8 | | | 0.33 | 63.0 |
| 1.0 ± 0.4* | | | | | | |

*Used in mouse trials.
†Solid in H₂O.
\hat{ }Diameter of the nanoparticle core and thickness of the shell in nm.
The relative error in the SAR measurements is ±8 relative percent.

TABLE VII
Solubility and SAR Values of Nanoparticle-
Ligand Combinations (5-7)

| | | | | Solu- | |
|---|---|---|---|---|---|
| | | | | bility | |
| | Li- | Li- | Li- | in H₂O | SAR |
| Fe/Fe₃O₄ | gand 5 | gand 6 | gand 7 | (mg/ml) | (W/g) |
| Fe: 5.4 ± 1.1* | 10 ± 6 | 108 ± 10 | | 0.35 | 63.9 |
| Fe₃O₄: 1.0 ± 0.4* | | | | | |
| Fe: 5.4 ± 1.1 | | 108 ± 10 | 10 ± 6 | 3.45 | 61.7 |
| Fe₃O₄: 1.0 ± 0.4 | | | | | |
| Fe: 5.4 ± 1.1 | 88 ± 18 | 10 ± 6 | 10 ± 6 | 2.87 | 62.4 |
| Fe₃O₄: 1.0 ± 0.4 | | | | | |
| Fe: 5.4 ± 1.1 | | 180 ± 25 | | 50.5 | 225 |
| Fe₃O₄: 1.0 ± 0.4 | | | | | |
| ASOX: 1.5 ± 0.5 | | | | | |
| Fe: 5.4 ± 1.1 | 10 ± 5 | 160 ± 20 | 10 ± 5 | 102 | 228 |
| Fe₃O₄: 1.0 ± 0.4 | | | | | |
| ASOX: 1.5 ± 0.5 | | | | | |
| Fe: 5.4 ± 1.1 | 20 ± 9 | 160 ± 20 | | 35 | 231 |
| Fe₃O₄: 1.0 ± 0.4 | | | | | |
| ASOX: 1.5 ± 0.5 | | | | | |
| Fe: 5.4 ± 1.1 | | 160 ± 20 | 20 ± 9 | 120 | 250 |
| Fe₃O₄: 1.0 ± 0.4 | | | | | |
| ASOX: 1.5 ± 0.5 | | | | | |
| Fe: 7.2 ± 1.3 | | 270 ± 45 | | 35.8 | 2,600 |
| Fe₃O₄: 1.0 ± 0.2 | | | | | |
| ASOX: 1.5 ± 0.5 | | | | | |
| Fe: 7.2 ± 1.3 | 13 ± 8 | 245 ± 40 | 13 ± 8 | 80 | 2,550 |
| Fe₃O₄: 1.0 ± 0.2 | | | | | |
| ASOX: 1.5 ± 0.5 | | | | | |
| Fe: 7.2 ± 1.3 | 25 ± 15 | 245 ± 40 | | 325 | 2,680 |
| Fe₃O₄: 1.0 ± 0.2 | | | | | |
| ASOX: 1.5 ± 0.5 | | | | | |
| Fe: 7.2 ± 1.3 | | 245 ± 40 | 25 ± 15 | 115 | 2,750 |
| Fe₃O₄: 1.0 ± 0.2 | | | | | |
| ASOX: 1.5 ± 0.5 | | | | | |

*Used in the mouse trials.

TABLE VIII
SAR Values of additional nanoparticle/ligand combinations
compared to commercial Fe particles

| Sample | SAR [W/g (Fe)] |
|---|---|
| Commercially Available Iron Oxide Sample¹ | 9.24 |
| Commercially Available Iron Oxide Sample² | 8.2 |
| Fe (4.1 ± 0.5 nm)/Fe₃O₄ (1.0 ± 0.2 nm) | 46.7 |
| dopamine-monolayer, 75 ± 10 ligands per particle | |
| Fe (4.1 ± 0.5 nm)/Fe₃O₄ (1.0 ± 0.2 nm) | 46.6 |
| Ligand 1 (75 ± 10) | |
| Fe (4.1 ± 0.5 nm)/Fe₃O₄ (1.0 ± 0.2 nm) | 45.8 |
| Ligand 1 (67 ± 7), Ligand 2 (8 ± 6) | |

¹Fe₃O₄ (Feridex ®; Bayer HealthCare).
²Fe₂O₃ (Ferrotech; Nashua, NH).

### Example 19

### Magnetic Resonance Imaging

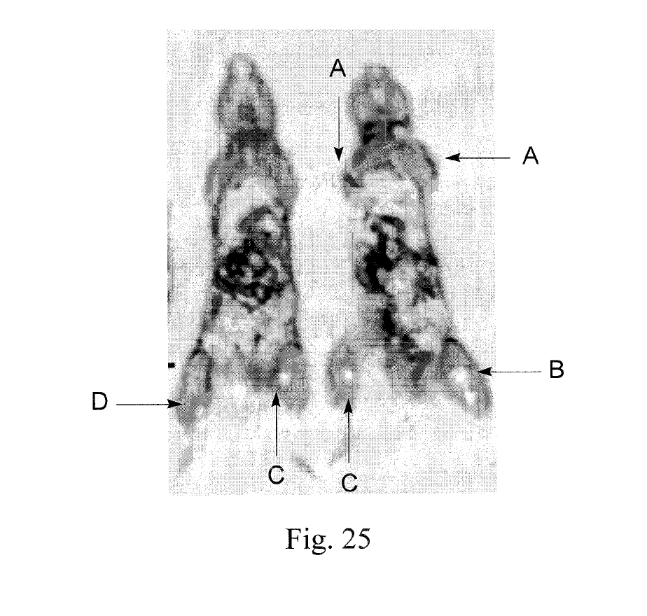

Two eight-week-old CB57BL/6 female mice (euthanized prior to this experiment) were injected with 0.50 ml of water (A) or magnetic nanoparticles (B-D). Site (B) contained 500 mg of stealth-coated Fe/Fe₃O₄ nanoparticles. Site (C) contained 25 mg of mouse stem cells, isolated from bone marrow that have been allowed to take up porphyrin-tethered stealth coated Fe/Fe₃O₄ nanoparticles. Site (D) contained 500 mg of commercially available iron oxide nanoparticles (Feridex®). MRI data was acquired using a Hitachi 7000 permanent magnet MRI. Standard T₁ and T₂ pulse sequences were used. As shown in the MR image in FIG. 25, except for the injection of water, discernible T₂ contrasts were obtained for all injections.

### Example 20

Hyperthermia Treatment of BF16F10 Melanomas in Charles River Mice

In this Example, the effect of the inventive nanoplatforms on Charles River mice with BF16F10 melanomas located in their upper hind legs was tested. Individual nanoparticles were used for these experiments (i.e., the nanoparticles were not linked by protease consensus sequences). Twenty mice with BF16F10 were innoculated with mouse melanoma cells in both of their upper hind legs, and then divided into four groups. Injections of the theranostic platforms were directly into the upper hind leg and proceeded as follows:
One group (“control right leg”) was injected with 50 μg stealth ligand-coated Fe/Fe₃O₄ nanoparticles featuring attached TCPP porphyrins, dissolved in 50 μL of PBS on day 6. On day 8, 100 μg of the nanoparticles in 100 μL of PBS were injected. On day 10, 150 μg of the nanoparticles in 150 μL of PBS were injected. Finally, on day 12, 150 μg of nanoparticles in 150 μL of PBS were injected.
The second group (“experimental right leg”) was injected according to the same injection schedule, followed by immediate hyperthermia treatment for 10 minutes. The temperature increased to 49.8° C. as confirmed by using a fiberoptic temperature measurement device (Neoptix).
The third group (“experimental left leg”) was injected with PBS (phosphate buffered saline) only and AC/magnetic irradiation was performed. The temperature increased to 42° C.
The forth group (“control left leg”) was untreated.

The mice were euthanized after day 14. Traces of the nanoplatforms were found in the lung, spleen, and liver (only minor traces). Most of the material (estimated to be more than 60 percent) was found as residual iron in the tumors themselves using Prussian blue staining.

The rate of cancer growth inhibition using the magnetic hyperthermia was 76% if the untreated melanomas are used as the control. The injection of the nanoplatform even without hyperthermia led to 50% inhibition of cancer growth, which can be attributed to biocorrosion of the nanoparticles and the iron (II/III)-enhanced chemistry of reactive oxygen species.

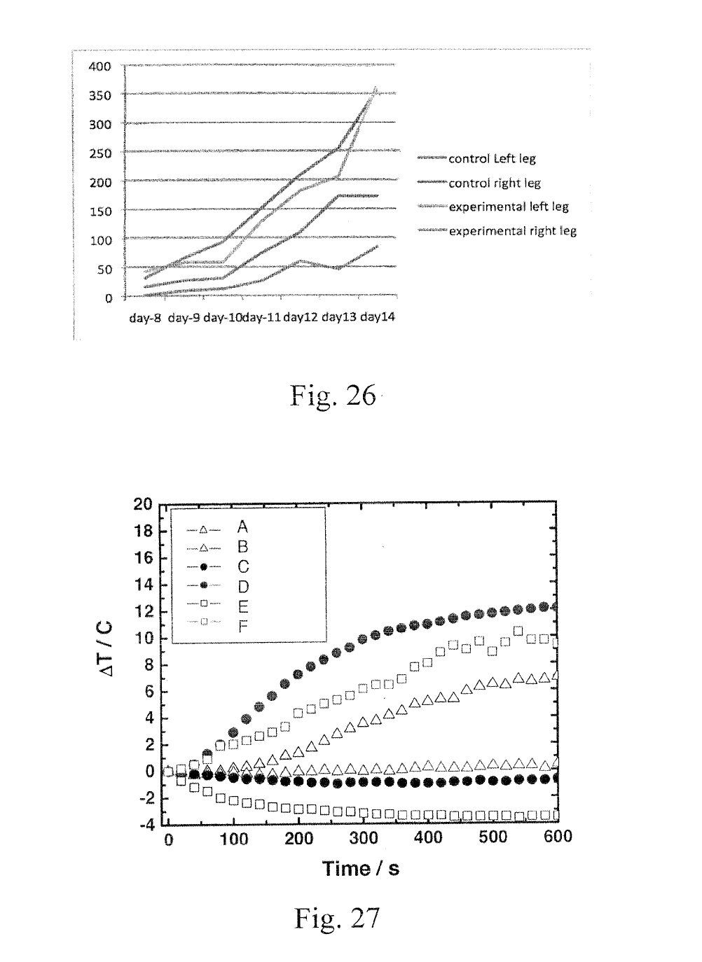

The average tumor volume (mm³) over time from the date of incubation of the tumor cells in the mice legs is depicted in FIG. 26. As can be seen in FIG. 26, the experimental right leg (nanoplatform followed by hyperthermia) had a significant inhibition of tumor growth when compared to the untreated group. The rate of growth inhibition using magnetic hyperthermia was 78%, if a further group that received 5 injections of PBS without hyperthermia is used as a control (graph not shown).

The nanoparticles featuring the porphyrin attachment were also injected intravenously into two other groups of mice to determine tumor uptake with this method of administering the nanoplatforms. One group was given, intravenously, 200 μg of the nanoplatform in 200 μl of PBS, while the other group was given, intravenously, 500 μg of the nanoplatform in 500 μl of PBS. The mice were euthanized and examined. Again, the majority (approximately 60%) of the administered nanoplatforms were found in the tumors 12 hours after injection.

### Example 21

### Magnetic Heating Experiments

In this Example, Charles River mice were injected with various solutions in the upper hind legs. The injection site was then heated using an A/C magnetic field (366 kHz, H, 5.0 kAm⁻¹). Unheated sites served as controls. The change in temperature (ΔT) over time (s) was monitored with a fiber-optic probe in the upper hind leg of the mice. The results are shown in FIG. 27. The test parameters were as follows:

TABLE IX
Test Parameters for In Vivo Magnetic Heating Experiments

| Sample | A/C Magnetic Field |
|---|---|
| A: 100 μl PBS | Yes |
| B: 100 μl PBS | No |
| C: 50 μg Fe/Fe₃O₄ in 100 μl PBS | No |
| D: 50 μg Fe/Fe₃O₄ in 100 μl PBS | Yes |
| E: 100 μg Fe₂O₃* in 200 μl PBS | No |
| F: 100 μg Fe₂O₃* in 200 μl PBS | Yes |

*Ferrotech (Nashua, NH).

### Example 22

### Calculation and Optimization of SAR Values

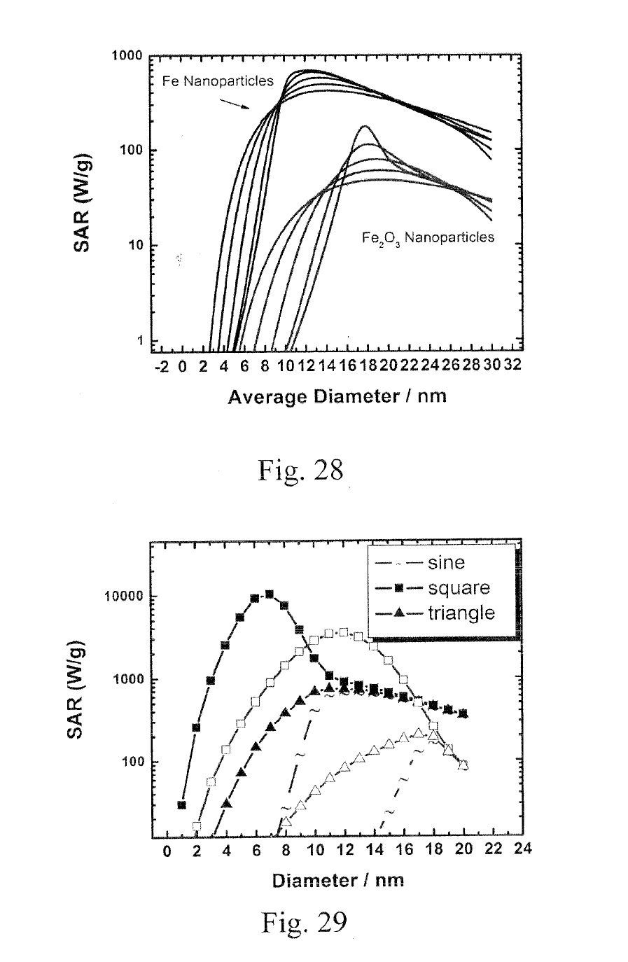

In this Example, theoretical calculations were performed to determine the effect of particle size and magnetic field shape on SAR values. First, the SAR values were calculated as a function of size based upon SAR=C*ΔT/Δt. Commercially-available Fe₂O₃ nanoparticles served as a reference. As shown in FIG. 28, the average size of the nanoparticle (diameter in nm) as well as the size distribution were found to significantly affect the SAR. The results show that for the 366 kHz magnetic hyperthermia apparatus, the optimum size distribution of the nanoparticles was approximately 10-12 nm for Fe nanoparticles, and 17-19 nm for Fe₂O₃ nanoparticles. The shallow curves correspond to subsequent broadening of the size distribution (σ=0-0.5 of a log normal size distribution) to account for more realistic experimental values. A narrower size distribution is desirable if the average nanoparticle size is close to desirable.

The effect of the shape (sine, triangular, square) of the magnetic field on the SAR values of Fe (black) and Fe₂O₃ (white) nanoparticles was also evaluated using theoretical calculations. A summary of the calculations is shown in FIG. 29. The calculations show that SAR values can be increased significantly if square magnetic fields are used (due to the increased contribution of Neel relaxation to the overall SAR values).

### Example 23

### In Vitro Nanodevice Data

In this Example, the SAR values, ΔTₘₐₓ, and solubility of various nanodevices were determined. Some of the nanoparticles in the nanodevices included aminosiloxane (ASOX) protecting layers, and/or biotin labels. Tetraethyleneglycol ligands were used. The ligands did not feature attached porphyrins. Magnetic heating was performed with a magnetic hyperthermia apparatus developed in-house using an A/C magnetic field (H, 5.0 kAm⁻¹, frequency 366 kHz (square wave pattern)). The apparatus uses a heavy duty induction heater converted to measure the temperature change of a sample, and remote fiber-optic sensing. The change in temperature was detected using a remote IR probe. Nanoplatform solubility was determined using the test described in Example 5 above. The results are presented in Table X below.

TABLE X
In Vitro Nanodevice Data

| | | | H₂O | |
|---|---|---|---|---|
| | Δtmax | Fe(0) Core | Solubility | SAR |
| Nanoparticle | (° C.)* | (nm)^{†} | (mg/ml) | (W/g) |
| Fe/Fe₃O₄ | 18 | 2.1 ± 0.4 | 0.015 | 24.5 |
| Fe/Fe₃O₄ | 25 | 4.1 ± 1.3 | 0.16 | 47.6 |
| Fe/Fe₃O₄ | 23 | 5.3 ± 1.2 | 0.11 | 46.4 |
| Fe/Fe₃O₄ | 34 | 5.4 ± 1.1 | 0.35 | 63.9 |
| Fe/Fe₃O₄/ASOX | — | 7.1 ± 1.1** | 85 | 2,200 |
| Fe/Fe₃O₄/ASOX- | — | 7.2 ± 1.1*** | 205 | 2,125 |
| biotin | | | | |
| Commercial Fe₂O₃^{‡} | 15 | 15 ± 3 | N/A | 4.32 |
| | | | (insoluble) | |

*Concentration: 0.050 mg/ml of stealth-coated nanoparticles. Fe concentration of 0.0107-0.1150 mg/ml (as determined by inductively coupled plasma (ICP)-fluorescence detection).
^{†}The thickness of the Fe₃O₄ on the invention nanoparticles is approximately 1.25 ± 0.25 nm.
**ASOX layer ± 2.1 nm.
***ASOX layer ± 2.5 nm.
^{‡}Ferrotech.

### Example 24

### Ligand Modeling

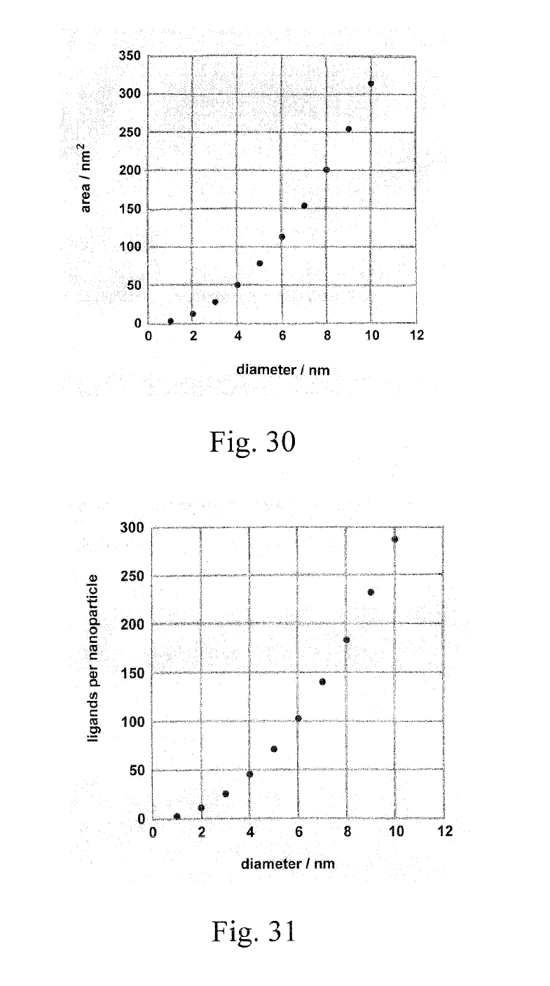

In this Example, calculations were performed to determine the suitable number of ligands for complete surface coverage of the nanoparticles. For the calculations, it is assumed that the nanoparticles are forms as perfect spheres where the surface area (A)=4πr²=dπ². The surface area of spherical nanoparticles as a function of their diameters is shown in FIG. 30.

The space demand of a dopamine unit, which is the “anchor” for the ligands of the invention has been calculated to be 1.094 nm². For the purposes of further calculation, it is assumed that each ligand has the same affinity towards surface binding so that the binding of multiple ligands to form a monolayer at the surface of the nanoparticle can be described as the Poisson distribution:

$$f ( k , λ ) = \frac{{λ}^{k} {e}^{- λ}}{k !}$$

where λ is the expected number of occurrences, k is the integer number of occurrences, and f is the probability of exactly k occurrences. FIG. 31 shows the ideal number of dopamin-anchored ligands per nanoparticle (for complete surface coverage) as a function of the nanoparticle diameter.

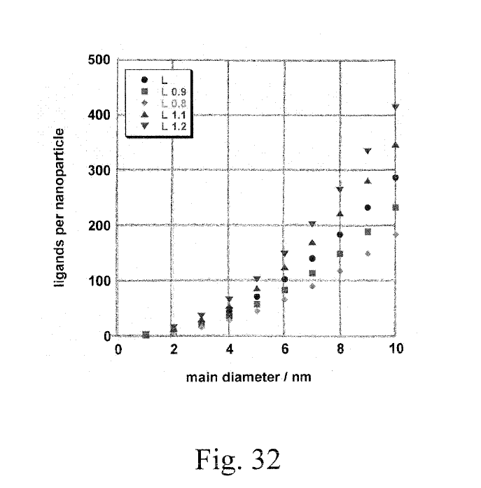

According to this devised model, the effect of variations in the nanoparticle diameter on the number of ligands that form a monolayer on the nanoparticle surface can be discerned. These results are shown in FIG. 32. L: main diameter as indicated; L 0.9: 90 relative % of the main diameter; L 0.8: 80 relative % of the main diameter; L 1.1: 110 relative % of the main diameter; and L 1.20: 120 relative % of the main diameter.

### Example 25

### In Vitro Monitoring of Treatment

In this Example, canine urine samples from dogs diagnosed with cancer and undergoing various stages of treatment were analyzed using the same general procedures outlined in Examples 13 and 14 regarding rat and mice urine. Three urine samples from canines were obtained from the Veterinary Medicine laboratory at Kansas State University. The samples were identified via code number and analysis was carried out without knowing the health status of each animal. The urine samples were collected and stored at −80° C. prior to the experiment. The experiment was carried out in 1 M PBS buffer (pH=7.2) at 35° C. To prepare the nanoplatform, TCPP was tethered via an oligopeptide containing a urokinase-specific cleavage sequence (SGRSA, SEQ ID NO: 2) to a dopamine-tetraethylene glycol ligand. This ligand was then bound to the Fe/Fe₃O₄-nanoparticles. The assembly was prepared using the same procedures described above in Example 12, except that only a non-metalated porphyrin was used. The TCPP-nanoparticle nanoplatform assembly was dissolved in the buffer using sonication for 30 minutes. The final concentration of nanoparticles in the solution was 15 mg/l. Next, 2 ml of the solution was taken to a fluorescence cuvette and the initial reading was recorded. To this solution 25 μl of each urine sample was added, mixed, and readings were recorded every 2 minutes.

The samples were then decoded and the results analyzed. Sample A was from a normal dog. Sample B was from a dog diagnosed with anaplastic sarcoma (2nd cancer), undergoing doxorubicin chemotherapy, and responding well to treatment. Sample C was from a dog recently diagnosed with renal lymphoma, and sick. The fluorescence signals generated after addition of dog urine samples were plotted against time. The plot of time versus the enhancement of fluorescence indicated the amount of urokinase present in each sample.

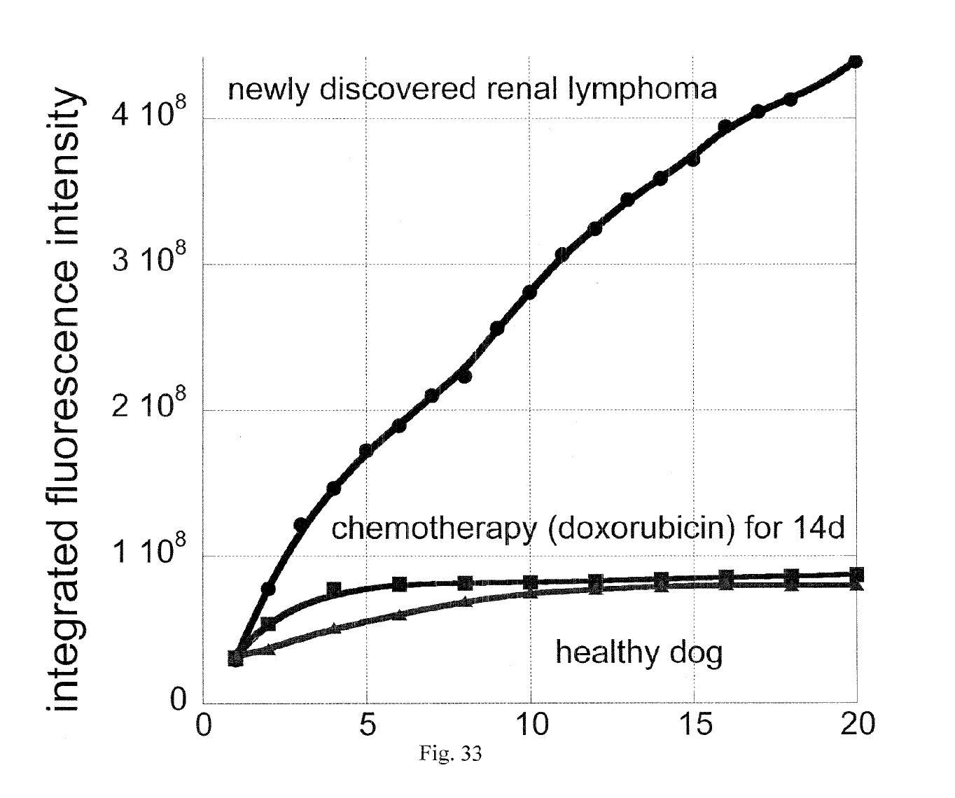

As shown in FIG. 33, the urine sample obtained from the dog just diagnosed with cancer (Sample C) showed a rapid increase in fluorescence, and the measurements were collected every one minute, indicating a greater enzyme hydrolysis rate compared to Other two samples which were only collected every 2 minutes. The urine sample from the dog undergoing chemotherapy (Sample B), had a detectable enhancement in fluorescence than the control (Sample A), but was still much lower than Sample C. Urine may contain fluorescent molecules that could excite in the 400-500 nm excitation wavelength range so it is important to analyze the urine sample by UV and fluorescence spectroscopy prior to the assays. The data indicates the ability of the assays to monitor and track progress of cancer treatment in vitro, based upon enzymatic activity levels.

### Example 26

### Stern Cell Delivery of Nanoplatforms

In this Example, stems cells were used to deliver the nanoplatforms to cancerous tissue.

1. Porphyrin-Tethered Stealth-Coated (Bi) Magnetic Fe/Fe₃O₄ Nanoparticles

Stealth-coated dopamine-labeled Fe/Fe₃O₄ nanoparticles featuring tethered TCPP were prepared by reduction of Fe(III) followed by formation of an aminosiloxane shell. The Fe/Fe₃O₄-core/shell nanoparticles were synthesized by NanoScale Corporation (Manhattan, Kans.). Addition of the organic stealth ligand in the presence of CDI attached an dopamine-anchored organic stealth layer around the aminosiloxane-layer. The final step consisted of the addition of TCPP-targeting units to the Fe/Fe₃O₄/ASOX/stealth-nanoparticles by reacting the terminal hydroxyl-groups of the tetraethylene glycol units with one carboxylic acid group of TCPP.

High Resolution Electron Microscopy (HRTEM) revealed that the nanoparticles are composed of nanorods (5-10 nm in length, 1-4 nm in diameter). After sodium-borohydride reduction, each nanorod contained an Fe(0)-core, as identified by HRTEM (lattice constant: 0.287 nm), and a Fe₃O₄ shell (thickness approx. 0.50-1.0 nm). The nanorods form clusters 16.0±1.5 nm in diameter. The nanoparticles had a BET surface area of about 72.2 m²/g, a BJH adsorption cumulative surface area of pores having a width between 17.000 Å and 3000.000 Å of 86.5 m²/g, and a BJH desorption cumulative surface area of pores having a width between 17.000 Å and 3000.000 Å of 91.1 m²/g. Phase analysis (powder X-ray diffraction-XRD) was determined using a powder X-ray diffraction (Shimadzu, XRD-6000) to determine the nanoparticles are nano crystalline or amorphous in structure. The XRD results are shown in FIG. 55, and show all the major lines for Fe₃O₄, as well as for the Fe core (along with amorphous iron oxide).

The synthesis of the aminosiloxane (ASOX) layer was performed by adapting a procedure from the literature: 20 mg of the Fe/Fe₃O₄ nanoparticles were suspended in 10 ml THF. After sonicating for 30 minutes, the undissolved solid (<1 mg) were separated by precipitation through low-speed centrifugation (1500 RPM, 5 min.). The clear solution was transferred to another test tube and 0.30 ml 3-aminopropyltriethoxylsilane was added to the solution, followed by sonication. The coated nanoparticles were then collected by high speed centrifugation (15,000 RPM for 15 min). After washing and redispersing in THF, the Fe/Fe₃O₄/ASOX-nanoparticles (7.5 mg) were collected, dried in high vacuum, and stored under argon. The thickness of the aminosiloxane shell surrounding the whole Fe/Fe₃O₄-clusters was 2.0±0.4 nm, which is consistent with an average diameter of the Fe/Fe₃O₄/ASOX-nanoparticles of 20±2.3 nm. Using the program IMAGE (NIH), the polydispersity index of the Fe/Fe₃O₄/ASOX-nanoparticles was determined to be 1.15.

The stealth ligand layer was synthesized by dissolving 40 mg dopamine-based ligand (L1) in 5.0 ml THF, along with 20 mg Fe/Fe₃O₄/ASOX nanoparticles and 1.0 g CDT added as a solid, followed by sonication. The nanoparticles were then collected by high speed centrifugation (15,000 RPM for 15 min.). After washing and redispersing in THF, the Fe/Fe₃O₄/stealth-nanoparticles (15 mg) were collected, dried in high vacuum, and stored under argon.

The porphyrin was attached to the nanoparticles by dissolving 2.5 mg of TCPP in 5.0 ml THF, along with 20 mg Fe/Fe₃O₄/ASOX/stealth nanoparticles, and 1.0/0.05 g EDC/HOST added as solids, followed by sonication. The porphyrin-attached nanoparticles were then collected by high speed centrifugation (15,000 RPM for 15 min.). After washing and redispersing in THF, the TCPP-labeled Fe/Fe₃O₄/ASOX/stealth-nanoparticles (13.5 mg) were collected, dried in high vacuum, and stored under argon. Using UV/Vis-spectroscopy (λ_{abs}(TCPP)=416 nm, =365,000 M⁻¹ cm⁻¹) it was determined that 5±0.5 TCPP units were bound to one stealth-coated Fe/Fe₃O₄/ASOX-nanoparticles on average. The stealth ligand had a length of 2.5 nm, so that the resulting Fe/Fe₃O₄/ASOX/stealth nanoparticles were 25±2.3 nm in size (diameter).

The space demand for the dopamine-anchor is 1.094 nm² (AM1). One Fe/Fe₃O₄/ASOX-nanoparticle of 20 nm in diameter can bind 1150 organic ligands. The porphyrin-labels have a diameter of 1.95 nm (AM1). The molar ratio of ligands L1/L1-TCPP was 1000/3.5. Assuming a Poisson distribution, 99.33% of the Fe/Fe₃O₄/ASOX/stealth-nanoparticles at the chosen ratio (5 TCPP units per nanoparticle) feature at least one chemically linked TCPP unit. The solubility of the organically coated Fe/Fe₃O₄ nanoparticles was determined to be 2.25 mg/ml, and the Specific Adsorption Rate (SAR) at the field conditions described here was 620±30 Wg⁻¹ (Fe). The zeta-potential of the Fe/Fe₃O₄/ASOX/stealth-TCPP nanoparticles was determined using Zeta Plus (Brookhaven instruments) to be 34 mV in 0.1 M PBS-buffer at 298K. The BET-surface area was determined to be 72±2 m² g⁻¹.

2. Tissue Culture of 017.2 Neural Stem Cells and B16-F10 Melanoma Cells

B16-F10 melanoma cells were purchased from ATCC (Manassas, Va.) and maintained in Dulbecco's Modified Eagle Medium (DMEM; Invitrogen, Carlsbad, Calif.) supplemented with 10% fetal bovine serum (FBS; Sigma-Aldrich, St Louis, Mo.) and 1% penicillin-streptomycin (Invitrogen) at 37° C. in a humidified atmosphere containing 5% carbon dioxide.

C17.2 neural stem cells (NSCs), a gift from V. Ourednik (Iowa State University; originally developed in Evan Snyder's lab), were maintained in DMEM supplemented with 10% PBS (Sigma-Aldrich), 5% horse serum (Invitrogen), 1% Glutamine (invitrogen), and 1% penicillin-streptomycin (Invitrogen).

3. Cytotoxicity of Fe/Fe₃O₄ Nanoparticles on Neural Stem Cells and B16-F10 Cells

Potential cytotoxic effects of Fe/Fe₃O, nanoparticles (NanoScale Corporation, Manhattan, Kans.) were studied by incubating C17.2 NSCs and B16-F10 melanoma cells with different concentrations of nanoparticles (as determined by iron content). NSCs and B16-F10 cells were plated at 50,000 cells/cm² and incubated overnight with their respective media containing nanoparticles at concentrations of 5, 10, 15, 20, or 25 μg/ml iron. After incubation, the media was removed and cells were washed twice with DMEM. Cells were lifted via trypsinization and live and dead cell numbers were counted via a hemocytometer and Trypan blue staining where viable cells appear colorless and non-viable cells are stained blue. NSCs and B16-F10 cells were used in three separate trials and each experiment was done in triplicate.

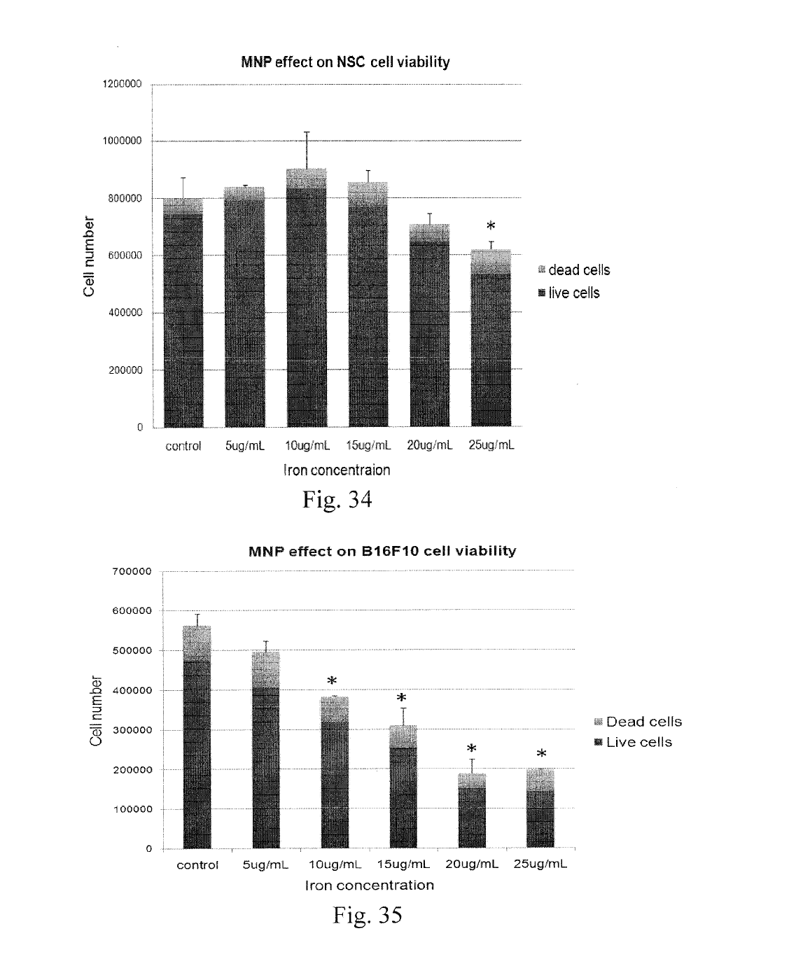

The toxic effect of the Fe/F₃O₄ nanoparticles increased with increasing iron concentration. Cell viability assessment for varying concentrations of Fe/Fe₃O₄ nanoparticles on NSCs is shown in FIG. 34 and on B16-F10 cancer cells is shown in FIG. 35. Interestingly, the Fe/Fe₃O₄ nanoparticles showed an increased toxic effect on B16-F10 cells compared to NSCs. NSCs tolerated the Fe/Fe₃O₄ nanoparticles well until 20 μg/ml iron concentration (FIG. 34). However, the B16-F10 cell number was decreased upon exposure to only 5 μg/ml iron concentration (FIG. 35).

4. Stem Cell Loading Efficiency and Strategy

The loading efficiency of the Fe/Fe₃O₄ nanoparticles into NSCs was assessed using Perl's Prussian Blue stain kit (Polysciences, Inc., Warrington, Pa.). After overnight incubation in NSC medium containing Fe/Fe₃O₄ nanoparticles (25 μg/ml Fe), the NSCs were washed twice with DMEM and PBS and fixed with 4% glutaraldehyde for 10 min. Fixed NSCs were incubated in 4% potassium ferrocyanide and 4% HCl for 20 minutes. After 20 min. incubation, the NSCs were washed twice with 1×PBS and counterstained with nuclear fast red solution for 30 minutes. Images were captured using a Zeiss Axiovert 40 CFL microscope (New York) and a Jenoptik ProgRes C3 camera (Jena, Germany).

The loading efficiency of NSCs with various iron concentrations of Fe/Fe₃O₄ nanoparticles was also determined spectrophotometrically using a Ferrozine iron estimation method (Riemer et al.; Coloimetric ferrozine-based assay for the quantitation of iron in cultured cells. Anal. Biochem. 331 (2) 370-75 (2004)). To estimate iron concentration per single cell, the total iron concentration of cells at each Fe/Fe₃O₄ nanoparticle concentration was divided by the total cell number. For this method, cells were incubated overnight with NSC medium containing different concentrations of Fe/Fe₃O₄ nanoparticles and then washed twice with DMEM and 1×PBS. All NSCs (control cells and cells loaded with various iron concentration of Fe/Fe₃O₄ nanoparticles) were trypsinized, centrifuged, and resuspended in 2 ml distilled water. Cells were then lysed by adding 0.5 ml of 1.2 M HCl and 0.2 ml of 2M ascorbic acid and incubating at 65-70° C. for 2 hours. After 2 hours, 0.2 ml of reagent containing 6.5 mM Ferrozine (HACH, Loveland Colo.), 13.1 mM neocuproine (Sigma-Aldrich, St Louis, Mo.), 2 M ascorbic acid (Alfa Aesar, Ward hill, MA) and 5 M ammonium acetate (Sigma-Aldrich, St Louis, Mo.) was added and incubated for 30 minutes at room temperature. After 30 minutes, samples were centrifuged at 1000 RPM for 5 minutes, and the supernatant optical density was measured by UV-VIS spectrophotometer (Shimadzu, Columbia, Md.) at 562 nm. A standard curve was prepared using 0, 0.1, 0.2, 0.5, 1, 2, and 5 μg/ml ferrous ammonium sulfate samples. Water with all other reagents was used as a blank.

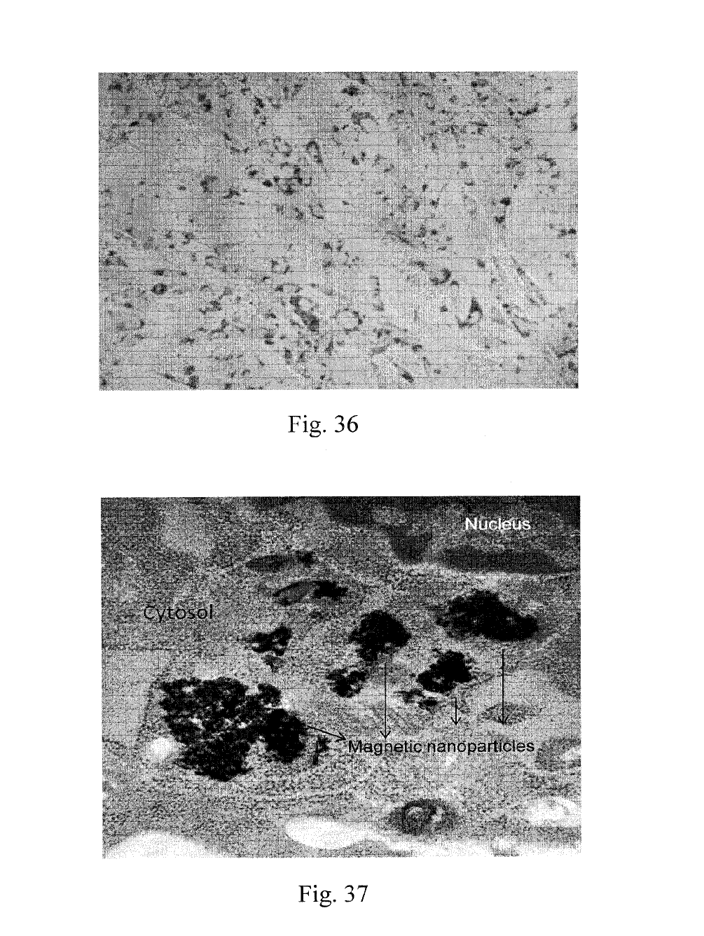

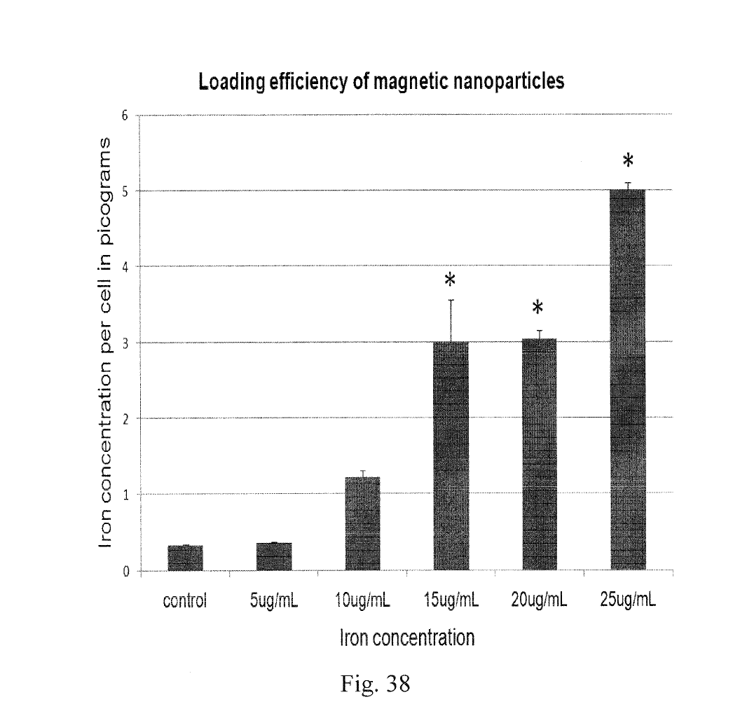

Fe/Fe₃O₄ nanoparticles efficiently loaded into NSCs after Prussian blue staining, Fe/Fe₃O₄ nanoparticles were detected in NSCs as blue staining material (FIG. 36). Electron microscope images of NSCs showed loaded Fe/Fe₃O₄ nanoparticles as aggregates in the cell cytoplasm (FIG. 37). More than 90% of the cells were loaded with Fe/F₃O₄ nanoparticles. The loading efficiency of Fe/Fe₃O₄ nanoparticles into NSCs increased with increasing concentration of Fe/Fe₃O₄ nanoparticles in medium. The highest concentration of 1.6 pg of iron per cell was identified in cells incubated with medium containing 25 μg/ml iron (FIG. 38).

The Fe/Fe₃O₄ nanoparticles may have appeared as aggregates rather than as single Fe/Fe₃O₄ nanoparticles in the cytoplasm of loaded cells because the porphyrin-tagged Fe/Fe₃O₄ nanoparticles may have clustered because they were adsorbed to fatty acids or hydrophobic proteins that were taken in by the LDL receptor. Clustering of the originally superparamagnetic particles may have changed their magnetic behavior to ferromagnetic.

5. AMF-Induced Temperature Changes In Vitro

To verify the temperature increase by NSCs loaded with Fe/Fe₃O₄ nanoparticles in a simulated tumor environment, NSCs were loaded overnight with Fe/Fe₃O₄ nanoparticles for a total Fe concentration of 15 μg/ml. It was not possible to insert the optical probe into actual melanomas because when this was attempted there was leakage of the gelatinous tumor parenchyma from the entry wound created by the probe. Hence, the tumor environment was mimicked by overlaying pelleted NSCs loaded with Fe/Fe₃O₄ nanoparticles or NSCs alone with agarose, which was allowed to gel in a micro centrifuge tube. After incubation, the loaded cells were washed twice with DMEM and twice with 1×PBS to remove free Fe/Fe₃O₄ nanoparticles. Cells were lifted with 0.1% trypsin-EDTA, and 1×10⁶ cells were pelleted by centrifugation in 2 ml centrifuge tubes. Next, 1.5 ml of 4% agarose solution was added on top of the cell precipitate to mimic the extracellular matrix in tumor tissues. Agarose centrifuge tubes containing pelleted NSCs without Fe/Fe₃O₄ nanoparticles were used as negative controls and were made as described above. The experiment was conducted in triplicate. Before each tube was exposed to AMF, two optical probes were inserted into the tube: one at the pellet, and the second one at the middle of the agarose solid. Tubes were exposed to AMF for 10 min., and the temperature difference over time was measured by the probes.

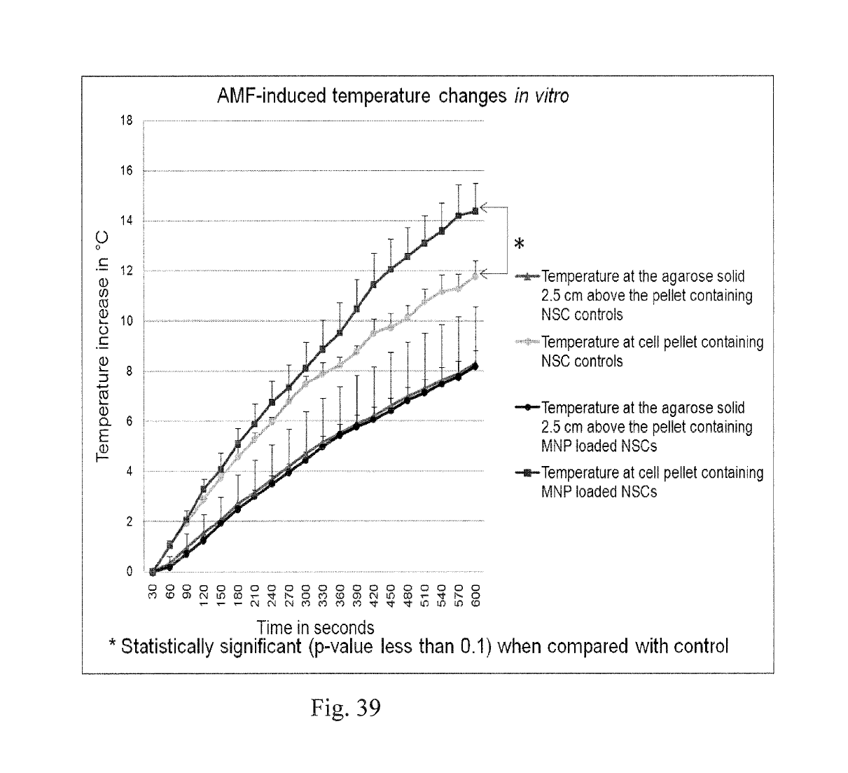

Temperature increase over time was compared between NSC controls and Fe/Fe₃O₄ nanoparticle-loaded NSCs (FIG. 39). There was a significant 2.6° C. increase in the pellet temperature between control and Fe/Fe₃O₄ nanoparticle-loaded cells (t-test, p-value 0.1) after 10 minutes AMF exposure time. Farther from the pellet in middle of agarose solid, there was a small temperature increase in both the groups due to residual heating; during AMF exposure the induction coil heats slightly and transfers its heat to the tube through air.

It is noteworthy that heating of the whole tumor region by using relatively large amounts of Fe/Fe₃O₄/ASOX nanoparticles may be unnecessary. Because of the very small Fe(0)-cores in the Fe/Fe₃O₄-clusters of nanorods, A/C-magnetic heating will mainly occur according to the Neel mechanism, resulting in the local heating of the nanoparticles. Larger nanoparticles (d>20 nm) feature the Brownian mechanism of heating, resulting in a much better stirring at the nanoscale level. The presence of the tetraethylene glycol units leads to a tight binding of water-molecules to the nanoparticles, which may further decrease the local diffusion. Therefore, “hot spots” featuring a temperature above 45° C. may exist during A/C magnetic heating, which can lead to local damage at multiple locations of the cells, even when the total temperature of the tumor tissue is not significantly enhanced.

6. Evaluation of Selective Engraftment of NSCs and Magnetic Hyperthermia

Female C57BL/6 (6-8 week old) mice were obtained from Charles River Laboratories (Wilmington, Mass.). Mice were held for 1 week after arrival to allow them to acclimate, and maintained according to approved IACUC guidelines in the Comparative Medicine Group facility of Kansas State University. All animal experiments were conducted according to these IACUC guidelines. On day 0, 3.5×10⁵ B16-F10 melanoma cells were injected subcutaneously into 21 C57BL/6 mice, and the mice were divided into three groups. On day 5, 1×10 NSCs loaded with Fe/Fe₃O₄ nanoparticles at 20 μg/ml iron concentration were injected intravenously to two groups (NSC-Fe/Fe₃O₄ nanoparticle, group I and NSC-Fe/Fe₃O₄ nanoparticle+AMF, group II); simultaneously, saline was injected into group III. On the 9th, 10th, and 11th days after tumor inoculation, group II mice with NSC loaded Fe/Fe₃O₄ nanoparticles were exposed to AMF for 10 min. daily using an alternating magnetic field apparatus (Superior Induction Company, Pasadena, Calif.). The frequency is fixed (366 kHz, sine wave pattern); field amplitude is 5 kA/m. Tumor volumes were measured using a caliper on days 8, 10, and 12; they were calculated using the formula 0.5aXb², where a is the larger diameter and b the smaller diameter of the tumor. All the mice were then euthanized on day 15 and the tissues were collected for histochemical studies.

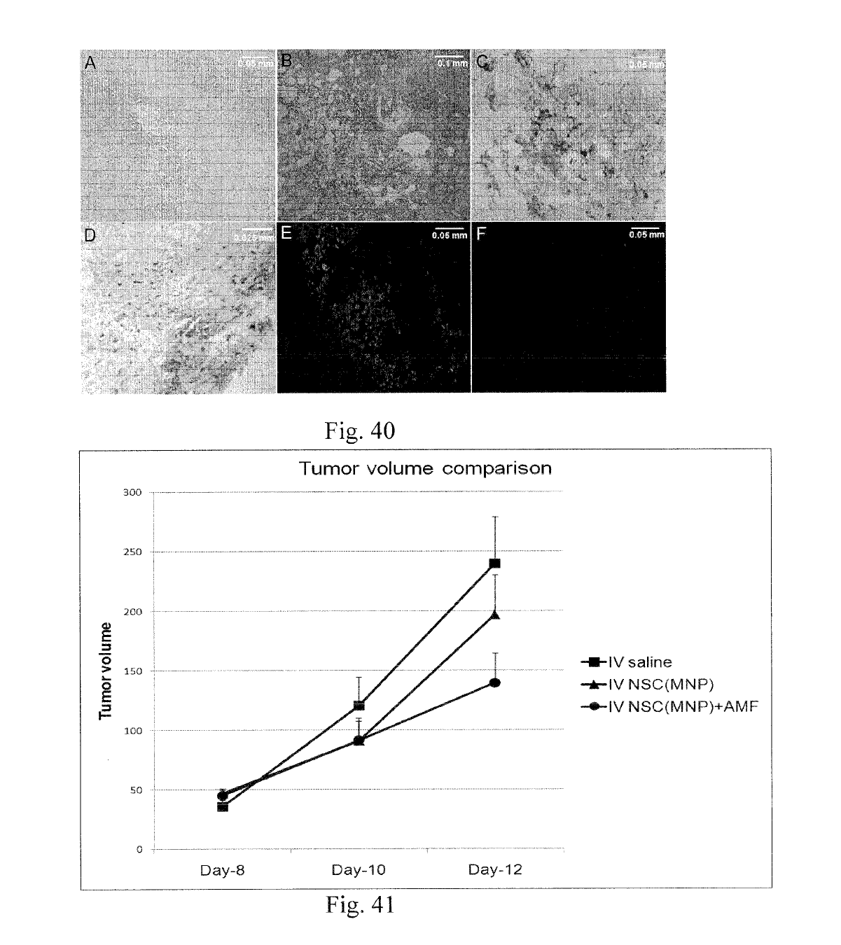

Significant numbers of Fe/Fe₃O, nanoparticle-loaded NSCs were identified in tumor sections 4 days after administration of cells. Images are provided in FIG. 40(A)-(F). (A)-(C): Prussian blue stained tissue sections, counterstained with nuclear fast red of lung (A), liver (B) and tumor (C) from mice which received nanoparticle-loaded NSCs followed by AMF treatment, note the absence of blue stained NSCs in the tumor sections. (D): Positive Prussian blue stained nanoparticle-loaded NSCs in tumor section of mice which received the nanoplatforms, but no AMF treatment. (E-F): TUNEL assay: Green apoptotic cells in tumor bearing mice with Fe/Fe₃O₄ nanoparticle-loaded NSCs+AMF (E) compared to few apoptotic cells in tumor bearing mice with saline only treatment (F). Tumor volume comparisons are graphed in FIG. 41. The smallest tumor volumes were observed in the group receiving NSCs loaded with Fe/Fe₃O₄ nanoparticles+AMF; the difference in tumor volume when compared with saline group was significant at day 12. There was no significant difference between tumor-bearing mice receiving NSC-Fe/Fe₃O₄ nanoparticle but no AMF and the saline group. There was tumor seepage after day 12 in the saline group due to increase in tumor sizes and hence the tumor volume measurements were not taken after day 12.

These results demonstrate that tumor-tropic stem cells loaded with Fe/Fe₃O₄ nanoparticles ex vivo and administered intravenously can result in regression of preclinical tumors after A/C magnetic field exposure. An advantage of the cell-based delivery of the Fe/Fe₃O₄ nanoparticles seems to be that it avoids agglomeration in the reticuloendothelial (mononuclear phagocytic) system, as seen with other delivery methods.

7. Histological Analysis

Tumor weights were measured to estimate tumor burden. Tumor, lung, liver, and spleen were snap-frozen in liquid nitrogen for histological analysis. Tissues were sectioned on a cryostat (Leitz Kryostat 1720, Germany) at 8-10 μm and used for IHC studies. Prussian blue staining was performed on these sections using Perl's Prussian blue stain kit to identify NSCs loaded with Fe/Fe₃O₄ nanoparticles. Apoptotic cell detection in the tissue sections was determined using the DeadEnd fluorometric terminal deoxynucleotidyl transferase dUTP nick end labeling (TUNEL) System (Promega Corporation, Madison, Wisc.), as per the manufacturer's protocol.

Although, Fe/Fe₃O₄ nanoparticle-loaded NSCs could be found near or within the tumor if no A/C magnetic field was administered, they were not found in tumors subjected to AMF exposure and evaluated at the end of the experiment. Prussian blue positive material also could not be found at the tumor site, indicating that the NSCs perished and released their cargo, which was subsequently removed from the site by phagocytic cells. The Fe/Fe₃O₄ nanoparticle-loaded stem cells themselves without A/C magnetic field exposure had a measurable but insignificant tumor inhibition effect. Another advantage with the stem cell-based approach was that the effects from biocorrosion and surfactant-release stay hidden within the delivering stem cells until they traffic to the tumor. Therefore, they will cause minimal damage elsewhere but will augment the hyperthermia effect in the tumors.

Tumors were collected 24 hours after the last AMF treatment on some of the mice to investigate potential mechanisms. The apoptotic index was found to have increased in the NSC-Fe/Fe₃O₄ nanoparticle IV transplanted group after three rounds of AMF, indicating that the targeted magnetic hyperthermia had a measurable effect on cell viability 24 hours after the last treatment. This corresponds to the time at which subcutaneous tumor volumes in the group receiving NSCs loaded with Fe/Fe₃O₄ nanoparticles and subsequent AMF were significantly less than tumor volumes in any of the other groups. Hence, apoptosis appears to be a mechanism involved in reduced tumor volumes

8. Protein Preparation for 2-Dimensional Electrophoresis (2-DE)

Total protein was prepared from melanomas isolated from mice given saline or NSC-Fe/Fe₃O₄ nanoparticle+AMF for use in two-dimensional gel electrophoresis (2-DE) analysis. The following protocol was used as previously described (Shevchenki et al., Mass spectrometric sequencing of proteins silver-stained polyacrylamide gels. Anal. Chem. 68 (5) 850-58 (1996)). Briefly, melanoma tissues were homogenized using Pellet Pestle Motor (KONTES, Vineland, N.J.) in the presence of 0.5 ml of lysis buffer (8 M urea, 2 M thiourea, 4% 3-cholamidopropyl-dimethylammonio-1-propane-sulfonate (CHAPS), 100 mM dithiothreitol (DTT), 25 mM Tris-Cl, and 0.2% ampholyte (pH 3 to 10) (Amersham Pharmacia Biotech, Piscataway, N.J.). The supernatant was collected and then precipitated using 2 volumes of ice-cold acetone. The final protein pellet was dissolved in 100 μl of the sample buffer (8 M urea, 2 M thiourea, 4% CHAPS, 100 mM DTT, 25 mM Tris-C, and 0.2% ampholyte (pH 3 to 10)). Protein concentrations were determined using a reducing agent-compatible and detergent-compatible protein assay kit (Bio-Rad, Hercules, Calif.).

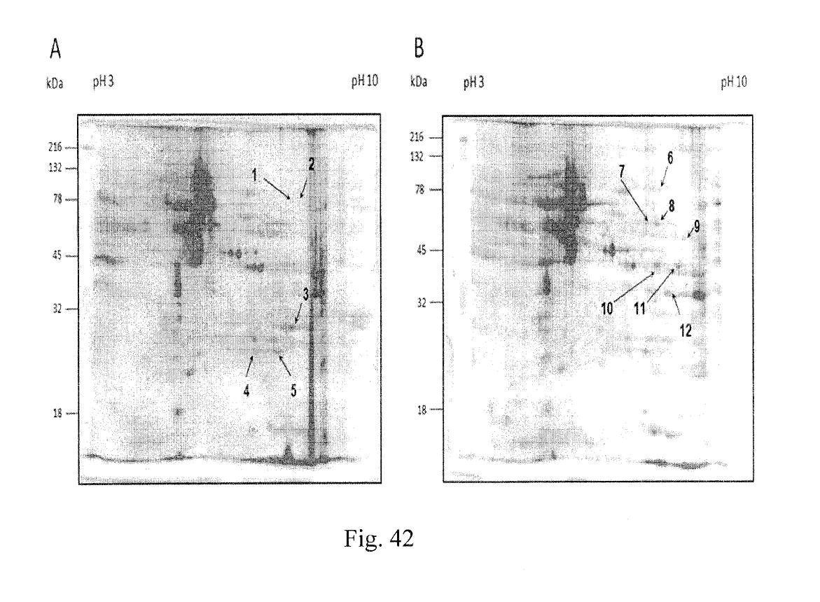

Gel spots representing 12 proteins expressed differentially in the 2 mouse groups were pinpointed using the MASCOT identification search software for identifying peptide mass fingerprinting (PMF). These protein spots are noted in FIG. 42(A)-(B). The protein samples were focused using 3-10 linear IPG strips for the first dimension, electrophoretically separated on 12% acrylamide gels, and stained with Biosafe Coomassie G-250 (company). Numbers with arrowhead lines refer to protein spots identified by MALDI-TOF analysis. An attempt was made to identify each of the proteins comprising the 12 differentially expressed spots using MALDI-TOF mass spectrometry. Identified proteins are listed in the Table in FIG. 43. As can be seen, phosphoglycerate kinase 1 (PGK-1) and neurotensin receptor 1 protein were much more highly expressed in tumors from the mice receiving intravenous NSC-Fe/Fe₃O₄ nanoparticle followed by AMF treatment than in the saline+AMF controls.

Of the seven protein spots found in the treated group but not the saline group (replicated four times; see the Table in FIG. 43), one candidate protein identified that could potentially exert an anti-tumor effect is phosphoglycerokinase-1 (PGK-1) which is an anti-angiogenic protein when over-expressed in some tumors. However, overexpression of PGK-1 in prostate cancer has been shown to facilitate tumor growth. On the other hand, there were five protein spots present in the saline control group that were not present in the treated group. One of these was TNF receptor-associated factor 5 (TRAF5), which is known to activate NF-kappaB. Another, biliverdin reductase B also increases NE-kappa B expression. NF-kappa B is a central player in transition to a more invasive state in some tumors. Biliverdin B was identified as a specific protein marker in microdissected hepatocellular carcinoma, elevated in methotrexate resistant colon cancer cells and is induced in renal carcinoma. Hence, it possible that down regulation of these genes could have been a factor in reduction of tumor size. While preliminary, these findings provide the background for further investigation to reveal potential mechanisms of tumor attenuation by AMF after targeted delivery of Fe/Fe₃O₄ nanoparticles by tumor-tropic stem cells.

9. Statistical Analysis

Statistical analyses were performed using WinSTAT (A-Prompt Corporation, Lehigh Valley, Pa.). The means of the experimental groups were evaluated to confirm that they met the normality assumption. To evaluate the significance of overall differences in tumor volumes and tumor weights between all in vivo groups, statistical analysis was performed by analysis of variance (ANOVA). A p-value of less than 0.1 was considered as significant. Following significant ANOVA, post hoc analysis using least significance difference (LSD) was used for multiple comparisons. Significance for post hoc testing was set at p<0.05. All the tumor volumes and weight data are represented as mean+/− standard error (SE) on graphs.

### Example 27

### Gold-Coated Nanoplatforms

In this Example, nanoplatforms were synthesized with a gold coating. Fe/Fe₃O₄/ASOX-nanoparticles were prepared by suspending 20 mg Fe/Fe₃O₄ nanoparticles in 10 mL THF. After sonicating for 30 minutes, the undissolved solid (<1 mg) was separated by precipitation through low-speed centrifugation (1500 RPM, 5 min.). The clear solution was transferred to another test tube and 0.30 ml 3-aminopropyltriethoxylsilane was added to the solution. After sonicating for 10 hours, the nanoparticles were collected by high speed centrifugation (15,000 RPM for 15 After re-dispersion and subsequent collection in THF (3×50 ml), the Fe/Fe₃O₄/ASOX-nanoparticles (7.5 mg) were collected, dried in high vacuum, and stored under argon.

Aminosiloxane-protected Fe/Fe₃O₄/Au-nanoparticles were prepared by pre-adsorbing Au(III) (0.50 mg of H[AuCl₄]) in aqueous medium to the terminal amino-functions of the Fe/Fe₃O₄/ASOX-nanoparticles. The nanoparticles were then collected by high speed centrifugation (15,000 RPM for 15 min.) and re-dispersed in ethanol. Depending on the thickness of the Au-shell that was desired, 2, 4, or 8 mg of H[AuCl₄] was then added, followed by sonication for 15 min. Au(III) was reduced to Au(0) by adding 5 mg of NaBH₄ at 20° C. The pre-seeding technique resulted in the formation of gold-shells. The Fe/Fe₃O₄/ASOX/Au-nanoparticles (14.0 g) were precipitated by centrifugation (15,000 RPM) and three times re-dispersed in and collected from water (3×50 ml), dried in high vacuum, and stored under argon. Due to clustering of the Fe/Fe₃O₄/ASOX/Au-nanoparticles, their hydrodynamic diameters were rather large. Typical values ranged from 550 nm to 750 nm with polydispersities in the range from 1.3 to 1.5. When adding surfactants (SDS, 0.01 M), the hydrodynamic diameters dropped to 200±20 nm.

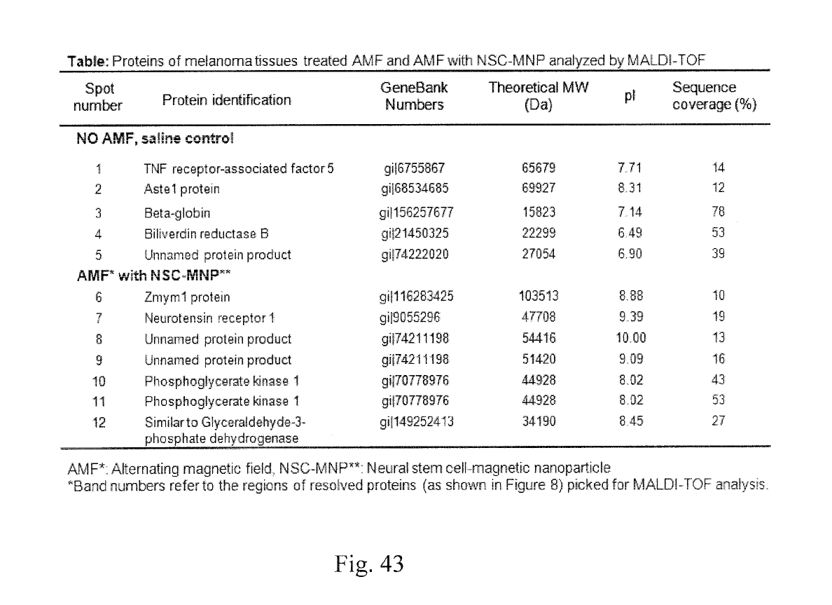

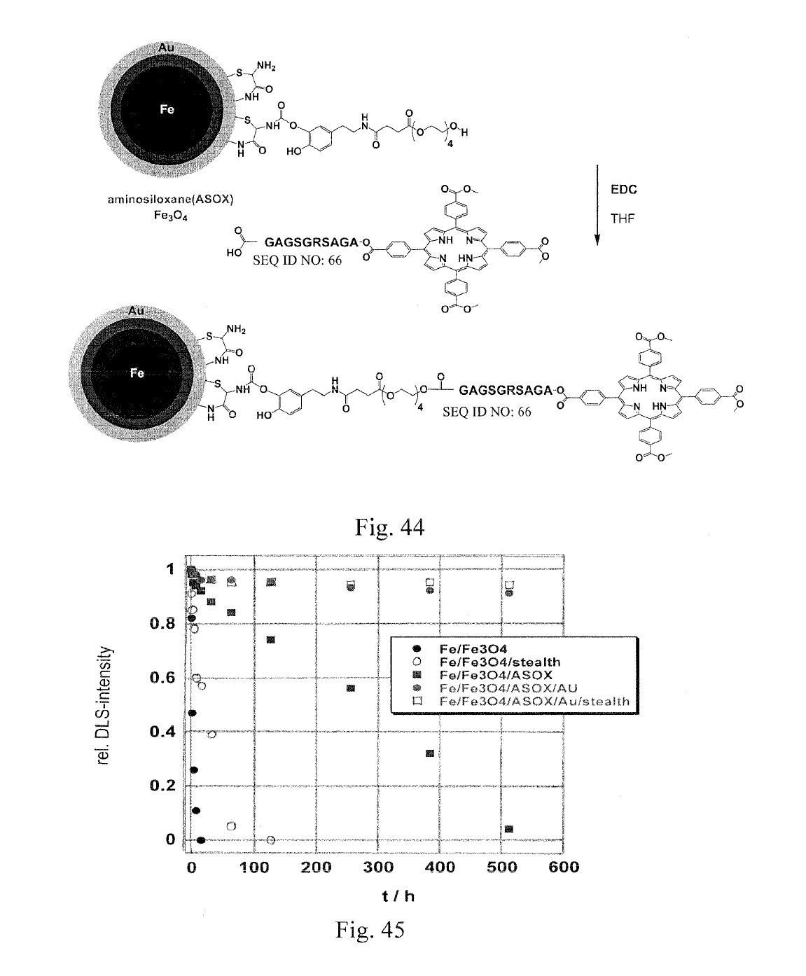

Fe/Fe₃O₄/ASOX/Au/stealth-nanoparticles were prepared by attaching a dopamine-based stealth ligand (see FIG. 44) to the Au-shell by a two-step approach: A) cysteinamide and Fe/Fe₃O₄/ASOX/Au-nanoparticles (10 mg) were allowed to react under sonication for 30 minutes in THF, followed by five consecutive precipitation (15,000 RPM) and re-dispersion procedures; B) the stealth ligand was then attached using the well established CDI-method in THF, followed by five consecutive precipitation (15,000 RPM) and re-dispersion procedures. The Fe/Fe₃O₄/ASOX/Au/stealth-nanoparticles (7 mg) were then dried in high vacuum, and stored under argon. The characterization of the nanoparticles is shown in Table XI.

TABLE XI
Nanoparticle Characterization

| | TEM-diameter | DLS-diameter | Solubility in | Polydisersity |
|---|---|---|---|---|
| Nanoparticle | (nm) | (nm) | H₂O (mg/ml) | Index (PDI) |
| Fe/Fe₃O₄ | 15 ± 1 | 102 ± 17 | 0.52 | 1.21 |
| Fe/Fe₃O₄/ASOX | 18 ± 1 | 101 ± 15 | 2.55 | 1.18 |
| Fe/Fe₃O₄/stealth | 23 ± 2 | 171 ± 21 | 1.88 | 1.22 |
| Fe/Fe₃O₄/ASOX/Au | clusters | 765 ± 105 | 0.05 | 1.34 |
| Fe/Fe₃O₄/ASOX/Au/stealth | 30 ± 2 | 188 ± 18 | 1.75 | 1.20 |

Stability tests were preformed using the five different nanoparticles (0.50 mg/ml) from Table XI above in aerated PBS-buffer. For the measurement of the Fe/Fe₃O₄/ASOX/Au/stealth-nanoparticles, 0.01M of SDS was added. The results are shown in FIG. 45. Unprotected Fe/Fe₃O₄-nanoparticles showed complete corrosion and chemical conversion to iron(II) and iron(III) salts/hydroxides within 16 hours. The addition of the organic stealth layer in Fe/Fe₃O₄/stealth-nanoparticles increased their half-life time from 4 hours (unprotected) to approximately 20 hours. The presence of the aminosiloxane protective layer on Fe/Fe₃O₄/ASOX-nanoparticles further increased the lifetime of the nanoparticles by an order of magnitude to 240 hours. Adding a second protective gold layer in the Fe/Fe₃O₄/ASOX/Au-nanoparticles caused a second increase to about 2,500 hours. Although the addition of the organic stealth layer in Fe/Fe₃O₄/ASOX/Au/stealth-nanoparticles greatly increased their solubility, it did not significantly affect their stability in aerated PBS.

Oligopeptides containing protease consensus sequences were synthesized in 250 mg batches using a microheterogeneous synthesis approach, starting with a Fmoc-Gly-Wang gel, followed by deprotection with piperidine/DMF (dimethylformamide) and coupling to the next Fmoc-protected amino acid using HBTU (2-(1H-Benzotriazole-1-yl)-1 1 3 3-tetramethyluronium) in DIEA (N,N-diisopropyl-ethylamine)/DMF. After the sequence was synthesized by step-by-step addition of further Fmoc-protected aminoacids, it was deprotected and separated from the Wang gel using TFA (trifluoroacetic acid). The sequences (purities>99%) are summarized in Table XII below.

TABLE XII
Sequences

| Protease | Oligopeptide |
|---|---|
| MMP-2 | GAGIPVS-LRSGAG (SEQ ID NO: 77, deleted by 3 residues from the N- |
| | terminus) |
| MMP-7 | GAGVPLS-LTMGAG (SEQ ID NO: 79, deleted by 3 residues from the N- |
| | terminus) |
| MMP-9 | GAGVPLS-LYSGAG (SEQ ID NO: 80, deleted by 3 residues from the N- |
| | terminus) |
| uPA | GAGSGR-SAGAG (SEQ ID NO: 66, deleted by 1 residue from the N- and C- |
| | termini |

The sequences were attached to the Fe/Fe₃O₄/ASOX/Au-nanoparticles and stealth-coated Fe/Fe₃O₄/ASOX/Au-nanoparticles, using TCPP as fluorescent dye and the same dopamine ligand linker as used for stealth coating. Three of the carboxylate groups on each TCPP were protected as methyl esters (available after column chromatography), and the TCPP was then attached via an amide bond to the terminal amino acid at the Wang gel prior to releasing the peptide. Coupling with the nanoparticles was carried out by forming an ester-linkage using EDC/HOBT, as described herein. This reaction scheme using dopamine ligand C (Example 1) and the Fe/Fe₃O₄/ASOX/Au-nanoparticles (no stealth coating) is shown in FIG. 44.

Time-resolved measurements can be used to demonstrate the “light switch” for cancer-related proteases. Emission results were obtained by time-correlated single photon counting. In the apparatus used in these studies, the sample was excited with approximately 15 nJ, 15 fs pulses from the second harmonic of a Ti:sapphire laser at a repetition rate of 80 MHZ. The excitation wavelength was fixed at 400 nm with excitation spot sizes of about 1 mm. This combination of low pulse energies and relatively large spot sizes results in power densities that are sufficiently low that multiphoton excitations are expected to be completely avoided. Detection was accomplished with a Hamamatsu 6μ MCP PMT and a time correlated single photon counting electronics. Wavelength selection was accomplished using interference filters. The instrument response function was determined by observing the laser scatter, and was about 60 ps FWHM. Polarized emission detection was accomplished using an emission polarizer in a perpendicular detection scheme relative to the excitation laser.

The nanoplatforms were prepared using the Fe/Fe₃O₄ nanoparticles, GAGSRGSAGAG linkage (SEQ ID NO: 66, deleted by 1 residue at each of the N-terminus and C-terminus), and non-metalated TCPP. The nanoplatforms were dispersed in PBS (0.1 μg/ml), followed by the addition of urokinase after 10 minutes. Free TCPP had a luminescence lifetime (monoexponential decay) of about 9 ns. In sharp contrast, Fe/Fe₃O₄-attached TCPP had a drastically shortened fluorescence lifetime due to the plasmon quenching effect of the nanoparticle. It was found that the presence of the gold plasmon added to the quenching effect of the nanoparticle. The overall fluorescence enhancement of this system was approx. 75 (10 min. after urokinase was added). Fluorescence lifetimes (and relative contributions (f) to the overall-decay with and without 1×10⁻⁷ M urokinase in PBS, are shown in Table XIII below.

TABLE XIII
Nanoplatform Fluorescence Lifetimes and
Relative Contributions to Overall-decay

| System | τ₁ (ns) | f₁ | τ₂ (ns) | f₂ |
|---|---|---|---|---|
| TCPP | 9.02 | 100 | — | — |
| Fe/Fe₃O₄-linkage-TCPP | 0.85 | 96 | 33.7 | 4 |
| Fe/Fe₃O₄-linkage-TCPP | 1.39 | 78 | 30.0 | 22 |
| plus urokinase | | | | |
| Fe/Fe₃O₄/ASOX/Au-linkage-TCPP | 0.70 | 98 | 29.8 | 22 |
| Fe/Fe₃O₄/ASOX/Au-linkage-TCPP | 1.47 | 80 | 29.3 | 20 |
| plus urokinase | | | | |

It can be seen from the observed lifetime-enhancement that TCPP becomes partially de-attached from the nanoparticle. It should be noted that the plasmon of the gold shell around Fe/Fe₃O₄ does only fluoresce a little.

Magnetic Heating, as previously described, was carried out using the gold-coated nanoparticles. The SAR rates were determined at 366 Hz and 100 kHz to determine their potential for different therapies. Although an A/C magnetic heating field of 366 Hz leads to larger heating effects, its tissue penetration is very limited, and therefore is primarily suitable for the treatment of melanomas and other surface tumors. 100 Hz is the established frequency for deep tissue applications. The results are provided in Table XIV below.

TABLE XIV
A/C Magnetic Heating Results

| | | SAR | SAR | |
|---|---|---|---|---|
| | TEM- | (W/g(Fe)) | (W/g(Fe)) | Fe-Content |
| Nanoparticle/ | diameter | 366 kHz, | 100 kHz | (weight %) |
| Nanoplatform | (nm) | 5 kA/m | 10 kA/m | from ICP* |
| Fe/Fe₃O₄ | 15 ± 1 | 570 ± 30 | 460 ± 15 | 42 ± 1 |
| Fe/Fe₃O₄/ASOX | 18 ± 1 | 2,250 ± 50 | 560 ± 20 | 34 ± 1 |
| Fe/Fe₃O₄/stealth | 23 ± 2 | 620 ± 30 | 530 ± 15 | 40 ± 1 |
| Fe/Fe₃O₄/ASOX/Au | clusters | 520 ± 25 | 450 ± 15 | 32 ± 1 |
| Fe/Fe₃O₄/ASOX/Au/stealth | 30 ± 2 | 500 ± 20 | 430 ± 20 | 28 ± 1 |

*Inductively Coupled Plasma with fluorescence detection.

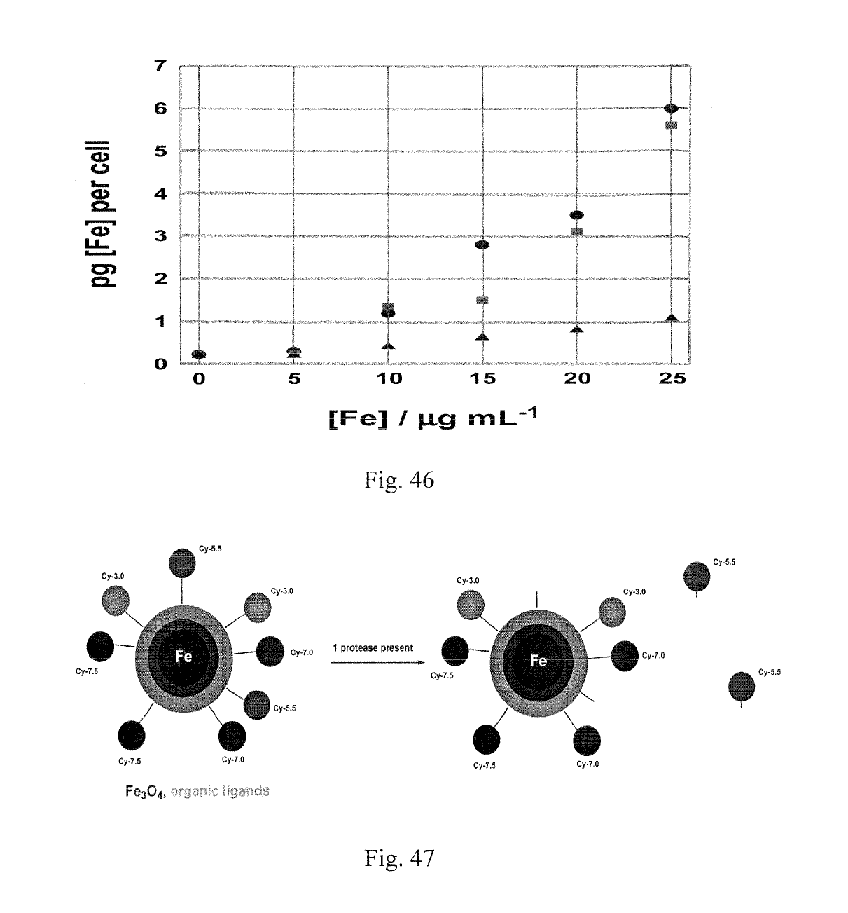

Cell loading and viability studies, as already described, were also carried out using the Au-coated nanoparticles. The cells were incubated for 24 hours with medium containing various nanoparticle concentrations. Fe/Fe₃O₄/stealth-nanoparticles featuring five chemically attached TCPP units were loaded into B16F10 melanoma cells, tumor-tropic NSCs, and MS-1 epithelial cells. More than 90% of the B16F10 melanoma cells and tumor-tropic NSCs cells were loaded with nanoparticles. The loading into MS-1 epithelial cells was less efficient by a factor of four. Fe/Fe₃O₄/ASOX/Au/stealth-nanoparticles possessing the same number of attached TCPP units were taken up much slower (by a factor of 20 and loaded very inefficiently). Since the Fe/Fe₃O₄/ASOX/Au/stealth-nanoparticles are distinctly bigger than Fe/Fe₃O₄/stealth (18 vs. 30 nm), the Au-coated nanoparticles may have exceeded the available pore-size for receptor-mediated cell uptake when using porphyrins as cell targeting moieties. After Prussian blue staining, MNPs were detected in all three cell types as blue staining material. The most efficient loading was seen in cells incubated with 25 μg/ml Fe concentrations. Loading efficiency is shown in FIG. 46.

### Example 28

### Nanoplatform Oligomers

In this Example, multiple nanoparticles were linked together to form nanoplatform oligomers (clusters) using a protease consensus sequence and ligand linkages between each particle. The oligomers are depicted in FIG. 49 using Fe/Fe₃O₄/ASOX/stealth-nanoparticles, GAGSGRSAGA (SEQ ID NO: 66, deleted at the N-terminus by 1 residue and the C-terminus by 2 residues) oligopeptide sequence, and dopamine linkages. The clusters can have any size between 1 and 20 nanoparticles, and could include any of the consensus sequences disclosed herein. Up to four cleavage sequences (e.g. uPA, MMP2, MMP9 and cathepsin D) could also be used in the cluster. MRI measurements were carried out in an NMR tube (400 MHz, Varian), 90 mol percent H₂O, 10 mol percent D₂O), as described, using 1 mL with an assay concentration (for urokinase) of 5 μg/ml, and T=298K. Before the measurement, the T/time of H₂O was 3.004 seconds, and the T₂ time was 0.07579 seconds. Next, 1×10⁻¹⁴ mol urokinase per ml was added in 1 ml H₂O/D₂O (90/10). After 10 minutes, T₁ had decreased to 2.003 seconds, and T₂ had increased to 0.1334 seconds.

### Example 29

### Monocyte/Macrophage Delivery

A mouse tumor-tropic monocyte/macrophage line (RAW264.7 Mo/Ma cells, American Type Culture Collection, Manassas, Va.) was loaded with biotin-tagged Fe/Fe₃O₄/ASOX-TCPP nanoplatforms to evaluate their potential for delivery to cancerous tissue. Monocytes are especially appealing in this capacity because they are autologous cells that can easily be obtained in large numbers for future human clinical trials. They will be cultured in their respective culture medium.

The uptake of siRNA-attached magnetic nanoparticles and SN38-attached magnetic nanoparticles has been analyzed for iron content using the ferrozine spectrophotometric assay (Riemer, et al. Colorimetric ferrozine-based assay for the quantitation of iron in cultured cells, Anal. Biochem. 2004, 331, 370-5) and by Prussian Blue staining (Shen et al. in vitro cellular uptake and effects of Fe₃O₄ magnetic nanoparticles on HeLa cells, Journal of Nanoscience and Nanotechnology 2009, 9, 2866-2871). Enough magnetic nanoparticles were added to the monocytes/macrophages or cancer cells to achieve 10, 15, 20, and 25 μg/ml Fe concentration in the media overnight. After incubation, the excess was removed by multiple washes of PBS. Cells were then evaluated for cytotoxic effects using the Cell Titer 96 Aqueous One Solution Cell Proliferation Assay, an MTS assay (Promega Corp., Madison, Wis.) to assess viable cell numbers. Loaded monocytes/macrophages were plated with PAN 02 cells (1:10 and 1:5 ratio) in narrow tissue culture “flat tubes,” 10 cm² surface area overnight followed by three media washes. These tubes can fit comfortably within the induction coil used to create the alternating magnetic field. They have been placed in the center of an RF coil (1 inch diameter, 4 turns) and treated at 10 kA/m, 100 kHz, sine wave pattern, for 30 minutes. Cell viability experiments were carried out 24 and 48 hours after treatment. All conditions were run in triplicate and replicated twice. In addition to the MTS assay, mitochondrial depolarization and cell viability were assessed quantitatively using the HCS mitochondrial health kit (Invitrogen Corp., Carlsbad, Calif.). Oxidative stress was also measured by detecting a decrease in reduced glutathione using the Thiol Tracker dye system (Invitrogen). Some wells were trypsinized, washed, and replated to assess the ability of the cells to re-attach and grow. FIG. 50 show the monocytes/macrophages loaded with the nanoparticles after 4 hours. The loaded cells appear blue because of the attached porphyrins.

### Example 30

### MRI Imaging

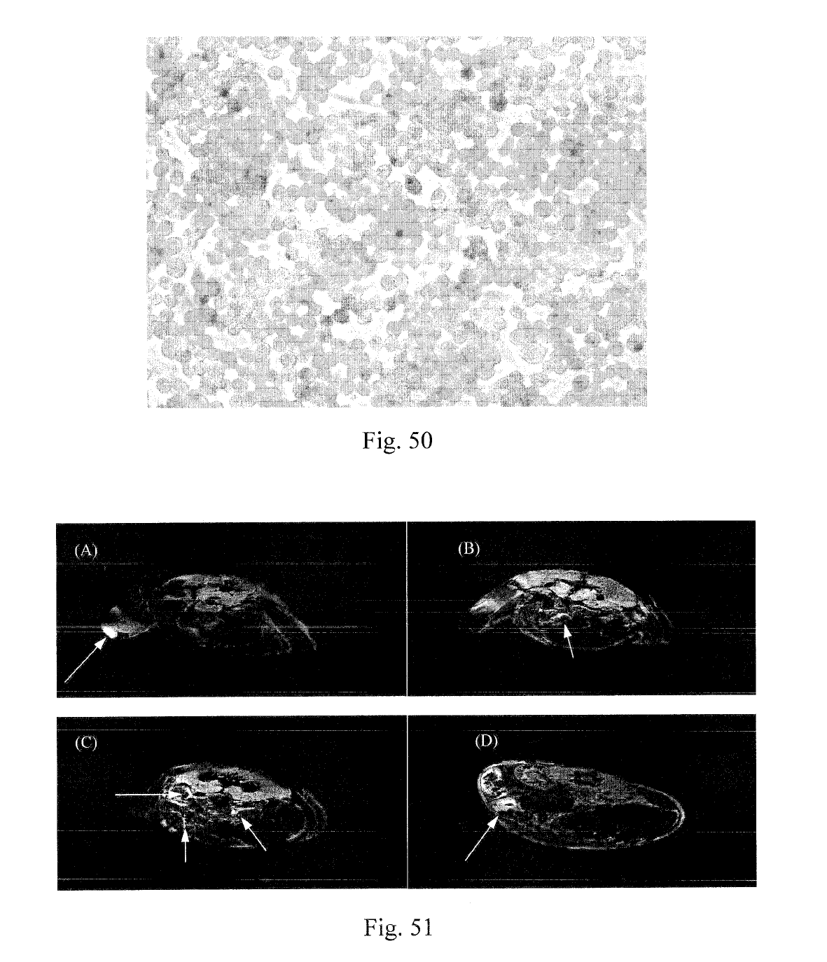

In this Example, the nanoplatforms were used as MRI imaging agents in C57/BL6 mice impregnated with B16F10 metastasizing lung melanomas. The Fe/Fe₃O₄/stealth nanoplatforms were loaded into NSCs and injected into the mice, and T₁-weighted images were collected at the Oklahoma Imaging Center MRI Facility using a 500 MHz NMR. Tissue containing the nanoparticles appears brighter in the images and indicated by the arrows. The images are shown in FIG. 51: (A) mouse cross-section, intramuscular injection of Fe/Fe₃O₄/stealth nanoparticles (50 micrograms); (B) lung melanoma nodes after stem cell delivery of the nanoparticles; (C) additional lung melanoma nodes; and (D) nanoparticles in the liver and kidney after stem cell delivery.

### Example 31

### Light Switch Imaging

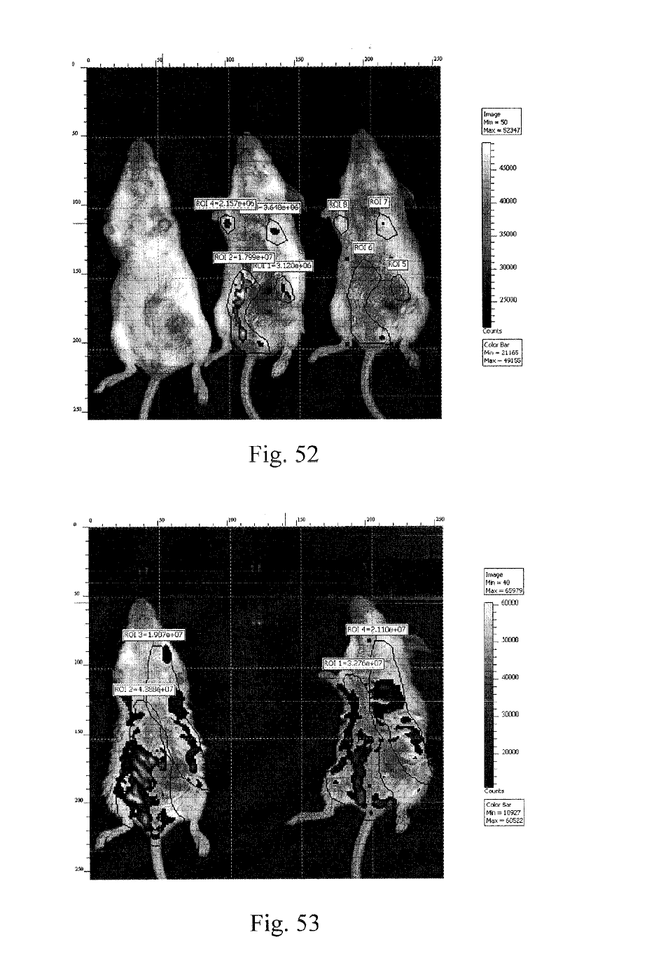

In this Example, the nanoplatforms were used to image cancerous tissue to demonstrate the usefulness of this method for tissue excision. Female BALB/c-mice that had been impregnated with metastastasizing 4T1 (aggressive breast cancer model) cancers were used for these studies. All three mice were impregnated into their mammary fat pads 18 days prior to imaging. The measurements were taken with the IVIS® Lumina imaging system from Caliper Life Sciences. The mice were anesthetized with isoflurane before and during the measurement. Fe/Fe₃O₄/stealth nanoparticles (d=16 nm, Fe core d=10 nm) featuring 30+/−5 cyanine 3.0 dyes per nanoparticle were used as the imaging nanoplatforms. A uPA cleavage sequence used was GAGSGRSAGA (SEQ ID NO: 66, deleted at the N-terminus by 1 residue and the C-terminus by 2 residues) for the oligopeptide linkage. The cyanine dye was very hydrophobic (log(octanol/water partition coefficient: 6.05)) (N1: —(CH₂)₅—COOH, N2: —C₈F₁₇), therefore the dye was deposited at the location of cleavage. One mouse served as the control. The second mouse received 5 mg of nanoplatform (3.1 mg total Fe) dissolved in 200 μl PBS injected directly into the tumor site. The third mouse received 1 mg of nanoplatform (0.62 mg total Fe) dissolved in 200 μl PBS injected directly into the tumor site. Images were taken 1 hour after injection, and are shown in FIG. 52 (left: control, middle: 5 mg nanoplatform, right: 1 mg nanoplatform). Excitation was performed at 535 nm using the IVIS 3D molecular imaging system from Caliper Lifesciences. Emission occurred at 565 nm (fluorescence maximum) The halo around the original cancer site is indicative of tissue infiltration by cancer cells. The results indicate that cyanine is cleaved and remains deposited at the cancer site, and is less prone to lymphatic drainage.

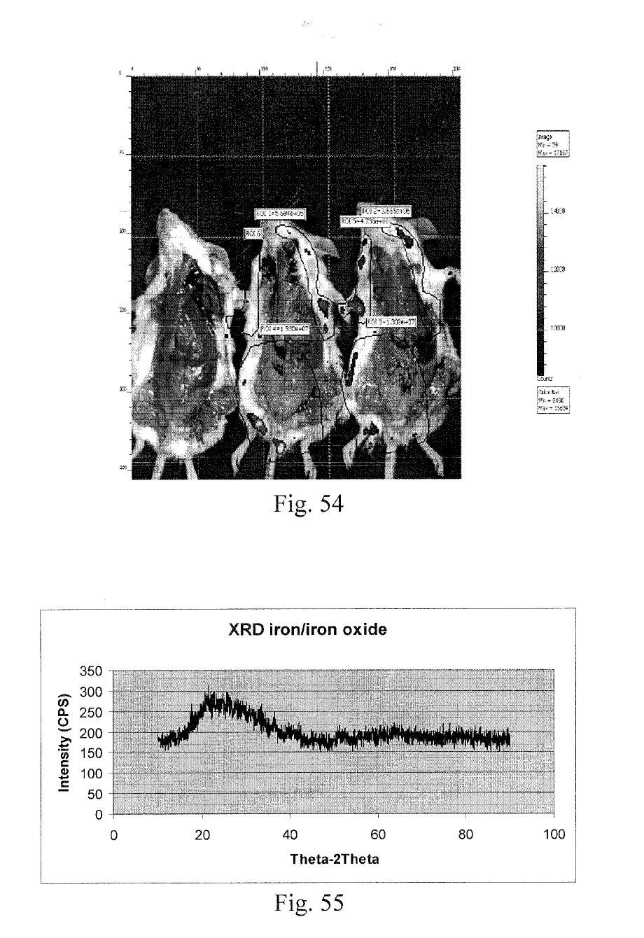

The above experiment was repeated using Fe/Fe₃O₄/stealth nanoparticles (d=16 nm, Fe core d=10 nm) featuring 30+/−5 TCPP dyes per nanoparticle attached via the same cleavage sequence as the imaging nanoplatform. Another nanoplatform was prepared using rhodamine B as the fluorescent dye. One mouse served as the control and received no injection. The second mouse received 5 mg of the TCPP nanoplatform (3.1 mg total Fe) dissolved in 200 μl PBS injected directly into the tumor site. The third mouse received 5 mg of the rhodamine B nanoplatform (3.1 mg total Fe) dissolved in 200 μl PBS injected directly into the tumor site. Images were taken 2 hours after injection. Excitation was performed at 480 nm with fluorescence of both TCPP and rhodamine B occurring in the integrated interval between 600 and 750 nm. The image of the TCPP and rhodamine B mice are shown in FIG. 53. As seen from FIG. 53, TCPP was transported through the lymphatic drainage pathways either due to is more hydrophobic nature (than cyanine) or because it binds to hydrophilic proteins that leave the cancer via the lymphatic drainage pathway. The same drainage was seen with rhodamine B. FIG. 54 shows images of the same mice, including the control, taken 24 hours after injection of the nanoplatforms. The dyes have been cleared from the lymphatic system, but remain in the metastasizing tumors. Guided by these images, a surgeon or oncologist could excise the tumors while preserving as much healthy tissue as possible.

1. A nanoplatform assembly for detecting protease activity comprising:
a first nanoplatform comprising a first nanoparticle and a protective layer;
a second nanoplatform comprising a second nanoparticle and a protective layer; and
an oligopeptide linkage between said first and second nanoplatforms, said linkage comprising a protease consensus sequence,
wherein at least one of said first or second nanoplatforms further comprises a functional group selected from the group consisting of porphyrins, chlorins, bacteriochlorins, phthalocyanines, biotin, derivatives thereof, and combinations thereof.

2. The nanoplatform assembly of claim 1, wherein said first nanoparticle and second nanoparticle are respective core/shell nanoparticles.

3. The nanoplatform assembly of claim 2, wherein each core is individually selected from the group consisting of Au, Ag, Cu, Co, Fe, and Pt.

4. The nanoplatform assembly of claim 3, wherein said core is a strongly paramagnetic Fe core.

5. The nanoplatform assembly of claim 2, wherein each shell is individually selected from the group consisting of Au, Ag, Cu, Co, Fe, Pt, the metal oxides thereof, and combinations thereof.

6. The nanoplatform assembly of 2, wherein said shell comprises iron oxide.

7. The nanoplatform assembly of claim 1, wherein said first and second nanoparticles have a Brunauer-Emmett-Teller multipoint surface area of at least about 20 m²/g.

8. The nanoplatform assembly of claim 1, said protective layers being individually selected from the group consisting of siloxane nanolayers, ligand monolayers, and combinations thereof.

9-19. (canceled)

20. The nanoplatform assembly of claim 1, wherein said protease consensus sequence is selected from the group consisting of SGRSA (SEQ ID NO: 2), VPMSMRGG (SEQ ID NO: 3), IPVSLRSG (SEQ ID NO: 4), RPFSMIMG (SEQ ID NO: 5), VPLSLTMG (SEQ ID NO: 6), VPLSLYSG (SEQ ID NO: 7), IPESLRAG (SEQ ID NO: 8), SGSPAFLAKNR (SEQ ID NO: 9), DAFK (SEQ ID NO: 10), SGKPILFFRL (SEQ ID NO: 11), SGKPIIFFRL (SEQ ID NO:12), GPLGMLSQ (SEQ ID NO:13), HGPEGLRVGFYESDVMGRGHARLVHVEEPHT (SEQ ID NO: 25), GPQGLAGQRGIV (SEQ ID NO: 26), SLLKSRMVPNFN (SEQ ID NO: 27), SLLIFRSWANFN (SEQ ID NO: 28), SGVVIATVIVIT (SEQ ID NO: 29), GAANLVRG (SEQ ID NO: 74), and PRAGA(SEQ ID NO: 75).

21.-22. (canceled)

23. A composition comprising a diagnostic assay including the assembly of claim 1 and a pharmaceutically-acceptable carrier.

24.-54. (canceled)

55. An MRI imaging method for detecting the activity of a protease associated with a cancerous or precancerous cell in a mammal comprising:
(a) administering to the mammal the composition of claim 23;
(b) locating said assay in a region of interest in the mammal suspected of having a cancerous or precancerous cell;
(c) transmitting radio frequency pulses to said region of interest; and
(d) acquiring MR image data of the region of interest, said MR image data comprising T₁ and T₂ values.

56.-60. (canceled)

61. The method of claim 55, said MR image data being T₂-weighted, said method further comprising detecting a change in the acquired T₂ values over time, said change corresponding to protease activity.

62.-66. (canceled)

67. An MRI imaging method for detecting the activity of a protease associated with a cancerous or precancerous cell in a mammal comprising:
(a) administering to the mammal a composition comprising a diagnostic assay including the nanoplatform assembly of claim 1, wherein said protease consensus sequence is SGRSA (SEQ ID NO: 2).
(b) locating said assay in a region of interest in the mammal suspected of having a cancerous or precancerous cell;
(c) transmitting radio frequency pulses to said region of interest; and
(d) acquiring a first MR image data of the region of interest, said first MR image data comprising T₁ and T₂ values.

68. The method of claim 67, wherein said first MR image data indicates protease activity, said method further comprising:
(e) administering to the mammal a composition comprising a diagnostic assay including the nanoplatform assembly of claim 1, wherein said protease consensus sequence is VPLSLTMG (SEQ ID NO: 6).
(f) locating said assay in a region of interest in the mammal suspected of having a cancerous or precancerous cell;
(g) transmitting radio frequency pulses to said region of interest; and
(h) acquiring a second MR image data of the region of interest, said second MR image data comprising T₁ and T₂ values.

69. The method of claim 68, wherein said second MR image indicates protease activity, said activity being correlated to a prognosis for angiogenesis or metastasis.

70. The method of claim 69, further comprising:
(i) administering to the mammal a composition comprising a diagnostic assay including the nanoplatform assembly of claim 1, wherein said protease consensus sequence is VPMSMRGG (SEQ ID NO: 3),
(j) locating said assay in a region of interest in the mammal suspected of having a cancerous or precancerous cell;
(k) transmitting radio frequency pulses to said region of interest; and
(l) acquiring a third MR image data of the region of interest, said third MR image data comprising T₁ and T₂ values.

71.-75. (canceled)

76. A nanoplatform comprising a first nanoparticle and a protective layer surrounding said nanoparticle, said protective layer being selected from the group consisting of siloxane nanolayers, ligand monolayers, gold coating layer, and combinations thereof.

77. The nanoplatform of claim 76, further comprising a functional group selected from the group consisting of porphyrins, chlorins, bacteriochlorins, phthalocyanines, biotin, derivatives thereof, and combinations thereof.

78. The nanoplatform of claim 76, said protective layer comprising a siloxane nanolayer, wherein said nanoplatform further comprises a ligand monolayer surrounding said siloxane nanolayer.

79. The nanoplatform of claim 78, further comprising a gold coating layer surrounding said ligand monolayer.

80. The nanoplatform of claim 76, wherein said nanoparticle is a core/shell nanoparticle, said core being selected from the group consisting of Au, Ag, Cu, Co, Fe, and Pt, and said shell being selected from the group consisting of Au, Ag, Cu, Co, Fe, Pt, the metal oxides thereof, and combinations thereof.

81. The nanoplatform of claim 80, wherein said core is a strongly paramagnetic Fe core.

82.-83. (canceled)

84. The nanoplatform of claim 76, wherein said nanoplatform is linked via an oligopeptide linkage to a particle selected from the group consisting of chromophores/luminophores, quantum dots, viologens, and combinations thereof, said oligopeptide linkage comprising a protease consensus sequence.

85.-91. (canceled)

92. The nanoplatform of claim 76, said nanoplatform being unlinked to any other nanoplatform.

93. (canceled)

94. A composition comprising a diagnostic assay including the nanoplatform of claim 76 and a pharmaceutically-acceptable carrier.

95.-97. (canceled)

98. A method of inhibiting the growth of cancerous or precancerous cells in a mammal comprising:
(a) administering to the mammal the composition of claim 94;
(b) locating said assay in a region of interest in the mammal suspected of having a cancerous or precancerous cell; and
(c) heating said nanoplatform using magnetic A/C-excitation, whereby the tissue in said region of interest is heated to a temperature of at least about 40° C., wherein said heating (c) results in apoptosis of said cancerous or precancerous cells.

99.-113. (canceled)

114. An MRI contrast agent comprising a core/shell nanoparticle having an iron core, said MRI contrast agent having an r₁ of greater than about 100 mM⁻¹s⁻¹ for T₂-enhancement and an r₂ with an integer number greater than about −2,000 mM⁻¹s⁻¹ for T₂-decrease.
